SEQUENCE LISTING (15 sequences)

SEQ ID NO: 1 - protein, 116 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 2 - protein, 111 aa, synthetic construct
DIQMTQSPSSLSASVGDRVTITCRASESVDNYGISFMNWFQQKPGGAPKLLIYAASNQGSGVPSRFSGSGSGTDFTLTISSLQPDDFATYYCQQSKEVPWTFGQGTKVEIK

SEQ ID NO: 3 - protein, 242 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGGAPKLLIYAASNQGSGVPSRFSGSGSGTDFTLTISSLQPDDFATYYCQQSKEVPWTFGQGTKVEIK

SEQ ID NO: 4 - DNA, 726 bp, synthetic construct
  source 1..726: mol_type = other DNA
caggtgcagctggtgcagtccggagcagaggtgaagaagcctggcagctccgtgaaggtgtcttgtaaggccagcggctataccttcacagactacaatatgcactgggtgcggcaggcaccaggacagggcctggagtggatcggctacatctatccttacaacggcggcaccggctataatcagaagtttaagtccaaggccaccatcacagccgatgagtctaccaacacagcctacatggagctgtctagcctgagatccgaggacacagccgtgtactattgcgcccggggcagacccgccatggattactggggccagggcaccctggtgacagtgtcctctggaggaggaggaagcggaggaggaggatccggcggcggcggctctgacatccagatgacccagtcccctagctccctgtctgccagcgtgggcgacagggtgaccatcacatgtagagcctccgagtctgtggataactatggcatcagcttcatgaattggtttcagcagaagcctggcggcgccccaaagctgctgatctacgcagcatccaaccagggatctggagtgccaagcaggttcagcggatccggatctggaaccgacttcaccctgacaatctctagcctgcagccagacgatttcgccacatactattgccagcagtctaaggaggtgccctggacctttggccagggcacaaaggtggagatcaag

SEQ ID NO: 5 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 6 - protein, 330 aa, Homo sapiens [sequence omitted]

SEQ ID NO: 7 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 8 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 9 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 10 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 12 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 13 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 6 aa, synthetic construct
HHHHHH

SEQ ID NO: 15 - protein, 8 aa, synthetic construct
HHHHHHHH

(19) United States
(12) Patent Application Publication
(10) Pub. No.: US 2026/0166145 A1
(43) Pub. Date: Jun. 18, 2026

(54) CD33 ANTIBODY COMPOSITIONS FOR TREATING ALZHEIMER’S DISEASE

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Yue-Ming Li, New York, NY (US); Wan Fung Eitan Wong, New York, NY (US); Manish Malviya, New York, NY (US); David A. Scheinberg, New York, NY (US); Kristen Vogt, New York, NY (US); George Liao, New York, NY (US)

(21) Appl. No.: 19/124,810

(22) PCT Filed: Oct. 25, 2023
(86) PCT No.: PCT/US2023/077722

§ 371 Date: Apr. 25, 2025

Related U.S. Application Data
(60) Provisional application No. 63/419,493, filed on Oct. 26, 2022.

(51) Int. Cl. A61K 40/41; A61K 31/13; A61K 31/192; A61K 31/27; A61K 31/445; A61K 31/496; A61K 31/506; A61K 31/55; A61K 38/28; A61K 39/00; A61K 39/395; A61K 40/31; A61K 40/33; A61P 25/28; C07K 14/47; C07K 14/725; C07K 16/28; C12N 5/0781; C12N 5/0783

(52) U.S. Cl. CPC A61K 40/41; A61K 31/13; A61K 31/192; A61K 31/27; A61K 31/445; A61K 31/496; A61K 31/506; A61K 31/55; A61K 38/28; A61K 39/0007; A61K 39/3955; A61K 40/31; A61K 40/33; A61P 25/28; C07K 14/47; C07K 14/7051; C07K 16/2803; C12N 5/0635; C12N 5/0636; C12N 2510/00

(57) ABSTRACT

The present disclosure provides methods for preventing or treating Alzheimer's disease in a subject, and/or reducing the likelihood or severity of Alzheimer's disease comprising administering to the subject an effective amount of an anti-CD33 antibody or an antigen binding fragment thereof. Alternatively, the methods comprise administering to the subject an effective amount of engineered immune cells expressing a neuronal antigen specific CAR and an anti-CD33 antibody or an antigen binding fragment thereof.

### CROSS-REFERENCE TO RELATED APPLICATIONS

This application is the U.S. National Phase Application of International Application No. PCT/US2023/077722, filed Oct. 25, 2023, which claims the benefit of and priority to U.S. Provisional Patent Application No. 63/419,493, filed Oct. 26, 2022, the entire contents of which are incorporated herein by reference.

### STATEMENT OF GOVERNMENT SUPPORT

This invention was made with government support under CA23766, CA55349, and CA08748, awarded by the National Institutes of Health. The government has certain rights in the invention.

### SEQUENCE LISTING

The instant application contains a Sequence Listing which has been submitted electronically in XML file format and is hereby incorporated by reference in its entirety. Said XML copy, created on Apr. 24, 2025, is named 115872-3248_SL.xml and is 20,480 bytes in size.

### TECHNICAL FIELD

The present technology relates generally to methods for decreasing, inhibiting or reducing brain beta amyloid (Aβ) accumulation or persistence in a subject in need thereof. The present disclosure also provides methods for treatment and/or prevention of Alzheimer's disease in a subject in need thereof. The methods of the present technology comprise administering to the subject an effective amount of an anti-CD33 antibody or an antigen binding fragment thereof. Alternatively, the methods disclosed herein comprise administering to the subject an effective amount of engineered immune cells expressing a neuronal antigen-specific CAR and an anti-CD33 antibody or an antigen binding fragment thereof.

### BACKGROUND

Alzheimer's disease (AD) is a progressive neurodegenerative disease most often associated with memory deficits and cognitive decline. It is estimated that there are approximately 44 million people worldwide living with AD or a related form of dementia. In the U.S., an estimated 5.5 million people of all ages have AD. Of these, around 5.3 million are 65 and older and 200,000 are younger and have early-onset AD. AD is a debilitating disease and there is no current effective treatment of preventative therapy.

AD is characterized by the deposition and accumulation of a 39-43 amino acid peptide termed the beta-amyloid protein, Aβ or β/A4 (Glenner and Wong, Biochem. Biophys Res Comm. 120:885-890, 1984; Masters et al, Proc. Natl. Acad. Sci. USA 82:4245-4249, 1985; Husby et al, Bull, WHO 71:105-108, 1993). Aβ is derived by protease cleavage from larger precursor proteins termed β-amyloid precursor proteins (APPs) of which there are several alternatively spliced variants. The most abundant forms of the APPs include proteins consisting of 696, 751 and 770 amino acids (Tanzi et al. Nature 31:528-530, 19980).

The small Aβ peptide is a major component that makes up the amyloid deposits or “plaques” in the brains of patients with AD. Accumulating evidence implicates amyloid, and more specifically, the formation, deposition, accumulation and/or persistence of Aβ fibrils, as a major causative factor of AD pathogenesis. It was discovered that the production of Aβ can result from mutations in the gene encoding its precursor, β-amyloid precursor protein (Van Broeckhoven et al, Science 248:1120-1122, 1990; Murrell et al, Science 254:97-99, 1991; Haass et al, Nature Med. 1:1291-1296, 1995). The identification of mutations in the beta-amyloid precursor protein gene that cause early onset familial AD is the strongest argument that amyloid is central to the pathogenic process underlying the disease. Several reported disease-causing mutations have been discovered which demonstrate the importance of Aβ in causing familial AD (reviewed in Hardy, Nature Genet. 1:223-234, 1992). To date, there is no existing effective prophylactic or therapeutic drugs for AD.

Taken together, these studies demonstrate that there is a need for developing therapeutic agents that reduce, eliminate and/or prevent fibrillary Aβ deposition, accumulation and/or persistence in the brains of mammalian subjects.

### SUMMARY OF THE PRESENT TECHNOLOGY

In one aspect, the present disclosure provides an engineered immune cell comprising: (a) a neuronal antigen-specific receptor and/or a nucleic acid encoding the neuronal antigen-specific receptor; and (b) an anti-CD33 antibody, or an antigen binding fragment thereof and/or a nucleic acid encoding the anti-CD33 antibody or antigen binding fragment, wherein the anti-CD33 antibody or antigen binding fragment includes an immunoglobulin heavy chain variable region (V_{H}) comprising SEQ ID NO: 1 and an immunoglobulin light chain variable region (V_{L}) comprising SEQ ID NO: 2. The engineered immune cell of the present technology may further comprise FoxP3 and/or a nucleic acid encoding FoxP3.

The neuronal antigen-specific receptor may be a T cell receptor, a native cell receptor, a non-native cell receptor, or a chimeric antigen receptor (CAR). In some embodiments, the anti-CD33 antibody or antigen binding fragment is secreted. Additionally or alternatively, in some embodiments, the nucleic acid encoding the anti-CD33 antibody or antigen binding fragment comprises a leader sequence for secretion of the anti-CD33 antibody or antigen binding fragment.

Additionally or alternatively, in some embodiments, the chimeric antigen receptor comprises (i) an extracellular antigen binding domain; (ii) a transmembrane domain; and (iii) an intracellular domain. In some embodiments, the extracellular antigen binding domain binds to the neuronal antigen and/or comprises a single chain variable fragment (scFv), such as a human scFv. Additionally or alternatively, in certain embodiments, the extracellular antigen binding domain comprises a signal peptide that is covalently joined to the N-terminus of the extracellular antigen binding domain. In certain embodiments, the transmembrane domain comprises a CD8 transmembrane domain. The intracellular domain may comprise one or more costimulatory domains. Examples of the one or more costimulatory domains include, but are not limited to a CD28 costimulatory domain, a CD3ζ chain, a 4-1BBL costimulatory domain, and any combination thereof.

Additionally or alternatively, in certain embodiments, the anti-CD33 antibody or antigen binding fragment is a scFv, optionally wherein the scFv comprises the amino acid sequence of SEQ ID NO: 3. In some embodiments, the nucleic acid encoding the anti-CD33 antibody or antigen binding fragment comprises SEQ ID NO: 4. The nucleic acid encoding the anti-CD33 antibody or antigen binding fragment may be operably linked to a promoter, such as a constitutive promoter, or a conditional promoter. In certain embodiments, the conditional promoter is inducible by binding of the neuronal antigen-specific receptor. Examples of neuronal antigens include, but are not limited to GD2, GD3, GM1, NCAM, integrin 3, Thy-1, CD44, EGFRvIII, or PDGFR.

In any of the preceding embodiments, the engineered immune cell is a lymphocyte, such as a T cell, a B cell, or a natural killer (NK) cell. In some embodiments, the T cell is a CD4+ T cell or a CD8+ T cell. The engineered immune cell may be derived from an autologous donor or an allogenic donor.

In one aspect, the present disclosure provides a mixture of polypeptides comprising a first polypeptide comprising FoxP3 and a chimeric antigen receptor that specifically binds to a neuronal antigen, and a second polypeptide comprising an anti-CD33 antibody, or an antigen binding fragment thereof, wherein the anti-CD33 antibody or antigen binding fragment includes an immunoglobulin heavy chain variable region (V_{H}) comprising SEQ ID NO: 1 and an immunoglobulin light chain variable region (V_{L}) comprising SEQ ID NO: 2. The mixture of polypeptides may further comprise a self-cleaving peptide located between FoxP3 and the chimeric antigen receptor. Examples of self-cleaving peptides include a P2A or a T2A self-cleaving peptide.

Additionally or alternatively, in some embodiments, the second polypeptide comprises a leader sequence for secretion of the anti-CD33 antibody or antigen binding fragment. In some embodiments, the second polypeptide comprises a scFv, optionally wherein the scFv comprises the amino acid sequence of SEQ ID NO: 3. Additionally or alternatively, in certain embodiments, the chimeric antigen receptor comprises (i) an extracellular antigen binding domain; (ii) a transmembrane domain; and (iii) an intracellular domain. In certain embodiments, the extracellular antigen binding domain binds to a neuronal antigen-specific receptor. Examples of neuronal antigens include, but are not limited to GD2, GD3, GM1, NCAM, integrin 3, Thy-1, CD44, EGFRvIII, or PDGFR.

Additionally or alternatively, in some embodiments, the extracellular antigen binding domain comprises a scFv. In certain embodiments, the transmembrane domain comprises a CD8 transmembrane domain and/or the intracellular domain comprises one or more costimulatory domains. Examples of the one or more costimulatory domains include, but are not limited to a CD28 costimulatory domain, a CD3ζ chain, a 4-1BBL costimulatory domain, and any combination thereof.

In one aspect, the present disclosure provides a nucleic acid encoding any and all embodiments of the mixture of polypeptides described herein. The nucleic acid encoding the mixture of polypeptides may be operably linked to a promoter, such as a constitutive promoter, or a conditional promoter. In some embodiments, the conditional promoter is inducible by binding of the neuronal antigen-specific receptor.

In another aspect, the present disclosure provides a vector comprising any and all embodiments of the nucleic acid disclosed herein. The vector may be a viral vector, a retroviral vector, or a plasmid. Also disclosed herein are host cells comprising any and all embodiments of the nucleic acids disclosed herein or any and all embodiments of the vectors disclosed herein.

In yet another aspect, the present disclosure provides a method for treating or preventing Alzheimer's disease in a subject in need thereof, comprising administering to the subject a therapeutically effective amount of any and all embodiments of the engineered immune cell.

In one aspect, the present disclosure provides a method for treating or preventing Alzheimer's disease in a subject in need thereof, comprising administering to the subject a therapeutically effective amount of an anti-CD33 antibody, or an antigen binding fragment thereof, wherein the anti-CD33 antibody or antigen binding fragment includes an immunoglobulin heavy chain variable region (V_{H}) comprising SEQ ID NO: 1 and an immunoglobulin light chain variable region (V_{L}) comprising SEQ ID NO: 2.

In another aspect, the present disclosure provides a method for reducing brain accumulation and/or persistence of Aβ plaques in a subject in need thereof, comprising administering to the subject a therapeutically effective amount of an anti-CD33 antibody, or an antigen binding fragment thereof, wherein the anti-CD33 antibody or antigen binding fragment includes an immunoglobulin heavy chain variable region (V_{H}) comprising SEQ ID NO: 1 and an immunoglobulin light chain variable region (V_{L}) comprising SEQ ID NO: 2.

Additionally or alternately, in some embodiments of the methods disclosed herein, the anti-CD33 antibody or antigen binding fragment comprises a Fc domain of an isotype selected from the group consisting of IgG1, IgG2, IgG3, IgG4, IgA1, IgA2, IgM, IgD, and IgE. In some embodiments, the anti-CD33 antibody or antigen binding fragment comprises an IgG1 constant region comprising one or more amino acid substitutions selected from the group consisting of N297A and K322A. In certain embodiments, the anti-CD33 antibody or antigen binding fragment comprises an IgG4 constant region comprising a S228P mutation. The anti-CD33 antibody or antigen binding fragment may be a monoclonal antibody, a chimeric antibody, a humanized antibody, or a bispecific antibody.

In any of the preceding embodiments of the methods disclosed herein, the Fc domain comprises a blood-brain barrier (BBB) target epitope. In certain embodiments, the BBB target epitope comprises an IgG constant region comprising a plurality of amino acid substitutions selected from the group consisting of: (a) N384L, Q386L, P387V, E388W, N389V, N390G, D413A, R416T, and N421W; (b) N384Y, Q386T, P387V, E388W, N389S, N390H, D413S, R416E, and N421Y; (c) N384Y, Q386T, P387E, E388W, N389S, N390Q, D413E, R416D, and N421H; (d) N384V, Q386T, P387P, E388W, N389A, N390L, D413L, R416E, and N421W; (e) N384L, Q386H, P387V, E388W, N389A, N390V, D413P, R416T, and N421W; (f) N384Y, Q386T, P387E, E388W, N389S, N390S, D413T, R416E, and N421F; (g) N384Y, Q386T, P387E, E388W, N389S, N390S, D413T, S415E, R416E, and N421F; (h) E380W, N384Y, Q386T, P387E, E388W, N389S, N390S, D413T, S415E, R416E, and N421F; (i) E380W, N384Y, Q386T, P387E, E388W, N389S, N390S, K392R, D413T, K414R, S415E, R416E, N421F, S424T and S426G; and (j) E380L, N384Y, Q386T, P387E, E388W, N389S, N390S, D413T, S415E, R416E, and N421F.

Additionally or alternately, in some embodiments of the methods disclosed herein, the anti-CD33 antigen binding fragment is selected from the group consisting of Fab, F(ab′)2, Fab′, scFv, and Fv. In certain embodiments, the scFv comprises the amino acid sequence of SEQ ID NO: 3.

In any and all embodiments of the methods disclosed herein, the anti-CD33 antibody or antigen binding fragment is conjugated to insulin, transferrin, an interleukin, albumin, a plasma protein, a lipoprotein, a RVG29 peptide, a Mini-Ap4 (apamin) peptide, a shark antibody, an antibody that targets insulin receptor, an antibody that targets interleukin receptor, or an antibody that targets transferrin receptor. In certain embodiments, the anti-CD33 antibody or antigen binding fragment is encapsulated by a nanoparticle or an exosome.

In any of the foregoing embodiments of the methods disclosed herein, the subject is suspected of having, is at risk for, or is diagnosed as having late onset Alzheimer's disease, early onset Alzheimer's disease, or intermediate onset Alzheimer's disease. In some embodiments, the subject exhibits one or more signs or symptoms of Alzheimer's disease. Examples of signs or symptoms of Alzheimer's disease include, but are not limited to: cognitive dysfunction or decline; memory loss; agitation; mood swings; impaired judgment; dementia; difficulty with abstract thinking; difficulty with familiar tasks; disorientation; diminished communication skills; repetitive speech or actions; impaired visuospatial abilities; impaired speaking, reading, and writing; withdrawal; depression; loss of recognition; loss of motor skills and sense of touch; delusions; paranoia; verbal or physical aggression; and sleep disorders. The subject may exhibit mutations in one or more genes selected from the group consisting of APP, PS1, PS2, APOE4, CD33, CLU, BIN1, PICALM, CR1, CD2AP, EPHA1, ABCA7, MS4A4A/MS4A6E and TREM2.

Additionally or alternately, in some embodiments of the methods disclosed herein, the anti-CD33 antibody or antigen binding fragment or engineered immune cell of the present technology is administered systemically, intravenously, subcutaneously, intraperitoneally, intradermally, iontophoretically, transmucosally, intrathecally, intramuscularly, intracerebrally, or intracerebroventricularly. In some embodiments, the methods of the present technology further comprise separately, sequentially or simultaneously administering at least one additional therapeutic agent to the subject. Examples of additional therapeutic agents include, but are not limited to, donepezil, galantamine, memantine, rivastigmine, memantine extended-release and donepezil (Namzaric), aducanumab, solanezumab, insulin, verubecestat, AADvac1, CSP-1103, and intepirdine.

In any and all embodiments of the methods disclosed herein, administration of the anti-CD33 antibody or antigen binding fragment or engineered immune cell of the present technology results in decreased cell surface expression of CD33 in microglia, increased Aβ uptake by microglia, and/or prolonged survival of the subject.

In another aspect, the present disclosure provides a method of preparing immune cells for adoptive cell therapy comprising: isolating immune cells from a donor subject; transducing the immune cells with any and all embodiments of the nucleic acids disclosed herein or any and all embodiments of the vectors disclosed herein; and administering the transduced cells to a recipient subject. In some embodiments, the donor subject and the recipient subject are the same or different. The immune cells isolated from the donor subject may comprise one or more lymphocytes, such as a T cell, a B cell, a tumor infiltrating lymphocyte, or a natural killer cell. In some embodiments, the T cell is a CD8⁺ cytotoxic T cell or a CD4⁺ T cell. The T cell may comprise a native T cell receptor (TCR), a non-native TCR, or a chimeric antigen receptor (CAR).

### BRIEF DESCRIPTION OF THE DRAWINGS

FIG. 1 shows the amino acid sequences of the V_{H} (SEQ ID NO: 1), V_{L} (SEQ ID NO: 2), and the scFv of the huM195 lintuzumab (SEQ ID NO: 3).

FIG. 2 shows the nucleic acid sequence (5′ to 3′) of the huM195 lintuzumab scFv disclosed in FIG. 1 (SEQ ID NO: 4).

FIGS. 3A-3J. Lintizumab (HuM195) and its single chain variable fragment (scFv) treatment increase Aβ42 uptake by phagocytic cells through CD33 degradation. FIG. 3A: Normalized Aβ42 ELISA absorbance signal from cell lysates after the indicated pre-incubation time with HuM195 or control IgG (1 μg/ml) (see material and methods). HuM195 treatment increases Aβ42 uptake by THP-1 and the maximal affect is seen after 4 hours. FIG. 3B: Aβ42 ELISA absorbance signal from Human monocyte lysate after 4-hour pre-incubation with HuM195 (1 μg/ml), IgG control (1 μg/ml), no treatment followed by 3 hours Aβ42 incubation. means±SEM; n=4, one-way ANOVA (**p-value<0.01). FIG. 3C: Schematic representation of the experimental design for continues live imaging of pHrodo-Aβ42 uptake. HuM195 antibody or scFv were pre-incubated for 4 hours prior to addition of pHrodo-Aβ42 (1 μM) and images were taken every 2 hours. FIG. 3D: Representative images from continues live imaging (brightfield and RFP channel) of human stem cells derived microglia at 0, 12, 24 and 36 hours after addition of pHrodo-Aβ42 (10× objective). FIG. 3E: SDS PAGE analysis of HuM195 antibody and purified HuM195 scFv under non-reducing conditions. FIG. 3F: Quantification of continues live imaging of pHrodo-Aβ42 uptake by stem cells derived microglia shown in fluorescent (A.U.) taken every 2 hours for 36 hours. Cells were pre-treated with either PBS (no treatment), control antibody (1 μg/ml), HuM195 (1 μg/ml), control scFV (1 μg/ml), HuM195 scFv (1 μg/ml) and Latrunculin (1 μM) for 4 hours. means±SEM; n=4. FIG. 3G: Uptake rate (fluorescence A.U./hour) calculated from the slopes of the linear phase from FIG. 3F. means±SEM; n=4, one-way ANOVA (****p-value<0.0001). FIG. 3H: THP-1 pHrodo-Aβ42 uptake rate (fluorescence A.U./hour) pre-treated with either PBS (no treatment), control antibody (1 μg/ml), HuM195 (1 μg/ml), control scFV (1 μg/ml), HuM195 scFv (1 μg/ml) and Latrunculin (1 μM) for 4 hours followed by addition of pHrodo-Aβ42 (1 μM) and images were taken every 2 hours. means±SEM; n=4, one-way ANOVA (**p-value<0.01). FIG. 3I: Western blot analysis of THP-1 cells treated with PBS, control antibody (Her2), HuM195, control scFV, HuM195 scFv with anti-CD33 and anti-hIgG. FIG. 3J: Flow cytometry analysis of surface CD33 of THP-1 cells treated 1, 4 and 24 hours with PBS (no treatment), HuM195 antibody, HuM195 scFv and unstained control and labeled with commercially available FITC conjugated anti-CD33 antibody.

FIGS. 4A-4I. HuM195 antibody but not HuM195 scFv incubation induces CD33 dimerization and phosphorylation, relaying transient downstream signaling. Human Phospho-Immunoreceptor Array detection of tyrosine phosphorylated immunoreceptor in lysates prepared from THP-1 cells incubated with PBS versus HuM195 antibody or PBS versus HuM195 scFv (FIG. 4A) for 1 min. The red rectangles highlight the spots corresponding to CD33 phosphorylation. FIG. 4B: Quantification of spots intensities of phosphor CD33 form A (FIG. 4C) Western blot analysis of downstream phosphorylation with HuM195 treatment on THP-1 cells incubated with PBS, control antibody, HuM195 antibody, control scFv and HuM195 scFv for 1 min. Blots are done with anti-SHP1, anti-SHP1-p (Y564), anti-CD33, anti-AKT, anti-AKT-p (S473), anti-AKT-p (T308), anti-hIgG and anti-His antibodies. FIG. 4D: Western blot analysis the transient phosphorylation signaling with HuM195 antibody treatment on THP-1 cells incubated with control antibody versus HuM195 antibody for 1 min, 6 hours and 24 hours. Blots are done with anti-CD33, anti-SHP1, anti-SHP1-p (Y564), anti-AKT, anti-AKT-p (S473), anti-AKT-p (T308) antibodies. Quantification of the ratio between band intensities of pSHP1(Y564)/SHP1 (FIG. 4E), AKT-p (S473)/AKT (FIG. 4F) and AKT-p (T308)/AKT (FIG. 4G) from E. means±SEM; n=2, one-way ANOVA (**p-value<0.01 and ***<0.005). FIG. 4H: Representative images of HL60 cells treated with PBS, HuM195, control antibody (Her2) or HuM195 scFv for 1 hour and Proximity ligation assay (PLA) was done using commercial mouse anti-CD33 primary antibodies conjugated with −/+DNA probe. FIG. 4I: Quantification of PLA spots per cells in 7 different slides. means±SEM; n=7, one-way ANOVA (****p-value<0.0001).

FIGS. 5A-5G. HuM195 treatment induces expression of immune related genes including IL33 which enhance phagocytic ability. FIG. 5A: Gene expression profile by qPCR of THP-1 cell treated with HuM195 or control antibody for 24 hours. mRNA levels were normalized for control antibody. Selected genes expression with signification changes between HuM195 versus control antibody: IL33 (FIG. 5B), CXCL2 (FIG. 5C) and SPP1 (FIG. 5D). means±SEM; n=3, T-test (*p-value<0.05 and **<0.01). FIG. 5E: Media IL33 from THP-1 cells treated with PBS control, control antibody of HuM195 for 24 hours measured with IL33 ELISA. means±SEM; n=3, one-way ANOVA (*p-value<0.05). FIG. 5F: Quantification of continues live imaging of pHrodo-Aβ42 uptake by THP-1 shown in fluorescent (A.U.) taken every 2 hours for 36 hours. Cells were pre-treated with either PBS(0), human IL33 (100, 10 and 1 ng/ml) for 1 hour. means±SEM; n=5. FIG. 5G: Uptake rate calculated from the slopes of the linear phase from FIG. 5E. means±SEM; n=5, one-way ANOVA (***p-value<0.005 and ****<0.0001).

FIG. 6. The mechanism of Lintuzumab (HuM195) treatment in AD: induces microglia activation and modifies inflammatory response. HuM195 antibody or scFv binds directly binds to CD33 result in internalization and degradation of the protein. This eliminates the inhibitory effect of CD33, thus activates microglia to increase Aβ clearance capabilities. Interestingly, HuM195 full-length antibody can temporarily activates CD33 phosphorylation via cell surface receptor dimerization, turning on the “off” switch in the cytosolic ITIM. This activation leads to cascade of downstream phosphorylation of SHP1 and modification of phosphorylation state in AKT, namely increasing phosphorylation at S473 site and de-phosphorylation at T307 site, suggesting HuM195 antibody initiates the inhibitory signaling of CD33. However, this inhibitory state disappears when CD33 is internalized and degraded leading to increase in Aβ uptake. In addition, the degradation of CD33 modifies the inflammatory response in microglia by increasing expression and secretion of cytokines and chemokines including IL33, CXCL2 and SPP1. Of which IL33 was implemented in stimulating microglia to enhance Aβ uptake. Thus, demonstrating the efficacy of HuM195 to directly activate microglia to uptake and clear Aβ and to facilitate an inflammatory response to further recruit and amplify the ability to clear the toxic aggregates.

FIGS. 7A-7J. Endotoxin free purified AP peptides does not stimulate immune reaction and has homogeneous characteristic of AP oligomers. FIG. 7A: Schematic representation of the purification steps for Aβ peptide from Clear Coli overexpression. Bacteria lysate with overexpressed His-Sumo-Aβ was initially loaded on Ni column where His-tag fused protein are bound, after elution sample was incubated with ULP for Sumo cleavage and dialyzed against Tris 25 mM 0 mM NaCl buffer. Cleaved sample containing separated His-Sumo and Aβ was loaded on an anion exchange column and eluted with NaCl gradient. FIG. 7B: Representative anion exchange elution histogram showing absorbance in 280 nm (blue line) and percentage of 1M NaCl buffer (red line). FIG. 7C: SDS PAGE with ULP uncleaved and cleaved in lane 1 and 2 respectively, green box are fractions from first peak, red box are fractions from second peak and yellow box are fractions from third peak. The second peak contains the purified Aβ peptide. FIG. 7D: Western blot analysis of purified A040 and Aβ42 with total anti-Aβ (W02) and C-terminal anti-Aβ (G2-10). FIG. 7E: Aβ42 ELISA for purified A040 and Aβ42. means±SEM; n=3, one-way ANOVA (**p-value<0.01). FIG. 7F: Alkaline phosphatase signal from HEK-blue cells (Invitrogen) after 24 hours incubation with LPS, purified A040 and Aβ42 in different concentrations. FIG. 7G: TNFα secretion from PMA treated THP-1 monocyte cell line after incubation with LPS, Clear Coli purified A040 and Aβ42 in different concentrations for 24 hours. FIG. 7H: AFM images of purified Aβ42 at 100 nM. FIG. 7I: Quantification of the height of particles from AFM images. FIG. 7J: pHrodo Red, succinimidyl ester conjugated to purified Aβ42 analyzed on unstained 16.5% SDS PAGE.

FIGS. 8A-8E. HuM195 single-chain variable fragment (scFv) and HuM195 antibody characterization and time course of CD33 degradation. (FIG. 8A) Histogram of HuM195 scFv size exclusion chromatography (Superdex 75). The retention time of the peak correlated with the standard globular protein size of 20.2 kDa. Equilibrium dissociation constant (Kd) of HuM195 antibody (FIG. 8B) and HuM195 scFv (FIG. 8C) measured on purified CD33-FC protein using Octet platform. (FIG. 8D) Competitive ELISA analysis of HuM195 scFv. Plate was coated with purified CD33-FC overnight and pre-incubated with increase amount of HuM195 antibody. HuM195 scFv or commercial mouse anti-CD33 (100 nM) with add to bind potential accessible purified CD33-FC. Secondary HRP conjugated antibodies were added (anti-human Fab for HuM195 scFv or anti-mouse IgG for mouse anti-CD33) and HRP signal were normalized to 0 nM HuM195 antibody pre-incubation. (FIG. 8E) Western blot of CD33 of THP-1 cells treated with PBS control, HuM195 antibody, and HuM195 scFv after 5 min, 1, 3, 6, 24 hours.

FIGS. 9A-9B. Engineered B-cells secrete anti-CD33 scFv and FC.scFv which recognize CD33 antigen on HL60 cells. FIG. 9A: Raji cells were transduced with retrovirus carrying either mCherry (control), scFv-T2A-mCherry, or FC.scFv-T2A-mCherry genes. Transduction efficiency was evaluated based on expression of mCherry, which was 70-98% of cells. Secretion of T2A tagged scFv and T2A tagged FC.scFv in the supernatant was confirmed by western blot using anti-T2A antibody. CD33-antigen binding of secreted scFv and FC.scFv was characterized by flow cytometry on HL60 cells. FIG. 9B: Left histogram: CD33 bound scFv and FC.scFv were detected using APC labeled anti-T2A secondary antibody. Right histogram: cell-secreted scFv and FC.scFv specifically bind to CD33 was confirmed by preincubation of HL60 cells with anti-CD33 hu.m195 antibody before binding of the cell-secreted antibodies. The green histogram shows CD33 staining using positive control hu.m195 antibody alone, which was detected using anti-human secondary antibody. The blue and red histograms showing decreased binding of scFv and FC.scFv to CD33 respectively, on HL60 cells preincubated with the hu.m195 antibody that blocked binding to CD33. This data confirms that the cell-secreted antibodies bind to the same antigen CD33 antigen.

FIGS. 10A-10B. Engineered cell-secreted anti-CD33 scFv enhances phagocytosis of Aβ42 by human macrophages in vitro. FIG. 10A: Human peripheral blood CD14+ monocytes were isolated and cultured in 96 well plate for one week in full RPMI media supplemented with Penicillin-Streptomycin, 10% human serum, human M-CSF, IL-2, IL-4 and IL-13. M2 macrophages were then incubated with different anti-CD33 or control antibodies (1 μg/ml) for four hours before adding pHrodo labelled Aβ42 (1 μM) to assay the phagocytosis effector function of the macrophages. Internalization of extracellular Aβ42 by macrophages was imaged every 30 min for 48 hours by a Cytatoin instrument. FIG. 10B: pHrodo uptake in macrophages was measured over time. The phagocytosed Aβ42 in the macrophages (shown in fluorescence arbitrary units on the Y axis) treated with different antibodies was followed over time. (X axis). Both the positive control anti-CD33 IgG antibody hu.m195 and the scFv derived from it, have significantly increased the phagocytotic effector function of the treated macrophages compared to control IgG1 or scFv and untreated macrophages. Latrunculin was used as an inhibitor of phagocytosis (negative control).

FIGS. 11A-11B. Comparative analysis of differentially secreted scFv by engineered T, B and 293F cells. Human primary T cells, EBV immortalized B cells, and a control 293F cells were transduced with retrovirus carrying CD33 scFv-T2A-mCherry genes. mCherry positive cells were sorted to have 100% scFv secreting population for each cell type. T and B cells were cultured in full RPMI media supplemented with Penicillin-Streptomycin, 10% human serum, whereas 293F cells were cultured in a recommended 293F serum free media. After 72 hours of culture in suspension, supernatants were collected to measure the concentration of secreted scFv proteins by direct ELISA using anti-T2A antibody. Previously purified scFv protein was used as a known concentration control for the ELISA experiments. FIG. 11A: Concentrations of scFv secreted by different cell types in ng/ml per one million cells. FIG. 11B: Binding of CD33 scFv proteins secreted by different cell types was confirmed by flow cytometry. One ml of supernatant from each cell type was incubated with 5×10⁵ HL60 cells for 30 min on ice. CD33 bound scFv was detected using APC labeled anti-T2A secondary antibody.

FIGS. 12A-12B. Construction and characterization of anti-CD33 scFv secreting CD4 T-cells. FIG. 12A: Retroviral vector genome: the scFv gene was inserted in frame into an SFG-gamma retroviral vector with a mCherry gene as a fluorescent marker. The T2A peptide sequence was used to link the scFv and mCherry genes. The retroviral control vector carries only the mCherry gene. LTR=long term repeat, Ψ=psi packaging element, αCD33 scFv=CD33-specific single chain variable fragment, T2A=2A self-cleaving peptide, mCherry=A red fluorescent protein. The CD4 T-cells transduced with retrovirus become mCherry positive (Red cells) and secrete scFv antibody in the supernatant that is detected by Western blot using an anti-T2A secondary antibody. FIG. 12B: Representative histograms show secreted scFv binding to CD33 to HL60 cells (Left). The scFv binding to CD33 was diminished in a competition assay where CD33 was first blocked with HuM195 antibody (right), reconfirming scFv specificity to CD33 antigen.

FIGS. 13A-13B. Anti-CD33 scFv treatment increase Aβ42 uptake by macrophages in vitro. FIG. 13A: Primary human CD14+ monocytes were differentiated into macrophages in a 12-well plate using cytokines M-CSF, IL4, and IL13 in culture media. Eight days later, cells were treated with either anti-CD33 scFv or control scFv (1 μg/ml each) for 4 hours before pHrodoGreen labeled Aβ42 (1 μM) was added to the cells to initiate phagocytosis. After 24 hours of incubation, cells were imaged using a fluorescence microscope to detect pHrodoGreen-positive macrophages (top). Flow cytometry analysis was performed on trypsinized cells to determine the Median fluorescence intensity of the phagocytosed pHrodoRed-Aβ42 (bottom) by macrophages. FIG. 13B: Quantification of continuous live imaging of pHrodoRed-Aβ42 uptake by human CD14+ cells shown in fluorescent (A.U.) taken every 2 hours for 48 hours. Cells were pre-treated with either PBS (no treatment), control antibody (1 μg/ml), HuM195 (1 μg/ml), control scFv (1 μg/ml), and Latrunculin (1 μM) for 4 hours. means±SEM; n=4.

FIGS. 14A-14B. Anti-CD33 scFv treatment increase Aβ42 uptake by phagocytic cells in vivo. FIG. 14A: Primary human CD14+ monocytes were differentiated into macrophages in suspension using cytokines M-CSF, IL4, and IL13 in culture media. Eight days later, cells were treated with either anti-CD33 or control antibodies (1 μg/ml each) for four hours before injecting them in mice. The pHrodoRed labeled-Aβ42 (1 μM) and 3 million macrophage/mouse were injected intra-peritoneally. Forty-eight hours post-injection, cells were isolated from the peritoneum cavity and assessed for pHrodoRed-Aβ42 phagocytosis by flow cytometry. FIG. 14B: Anti-CD33 scFv or its complete IgG form (HuM195) treated cells had a higher degree of phagocytosis than control antibody-treated macrophages. Reconfirming these antibodies enhances phagocytosis in vivo (left). Median fluorescence intensity of the internalized pHrodoRed-Aβ42 by macrophages (right).

FIGS. 15A-15B. Construction and characterization of anti-GD2 CAR Tregs. FIG. 15A: Schematic of retroviral vectors encoding the GD2-targeted CAR gene with FoxP3. A T2A element sequence was used to link CAR with FoxP3. A c-terminal Myc-tag was added to detect exogenous FoxP3. A GD2 CAR construct without the FoxP3 gene was generated as a control of GD2 CAR Tregs (Top). The transduction efficiency of GD2 CAR expression on transduced human CD4 T cells was between 60-95%, as determined by CAR and Myc co-staining (bottom). FIG. 15B: Cytolytic activity of GD2 CAR-Tregs vs. GD2 CAR Tconv cells against SK-N-Be2 (GD2+) target cells expressing firefly luciferase (24 h, bioluminescence assay) (Left). GD2 CAR Tregs mediate antigen-specific immunosuppression against GD2 CAR Tconv cells in vitro. GD2 CAR Tregs mixed with GD2 CAR Tconv cells at different ratios and activated with GD2 antigen and FLUC expressing SK-N-Be2. Antigen-specific suppression of GD2 Tconv cell's target killing was measured by firefly luciferase (24 h, bioluminescence assay (Right). Retinoic acid and TGFβ treated CAR Tregs showed better suppressive and lower cytotoxic phenotypes.

FIG. 16. Construction and characterization of dual virus transduced scFv secreting GD2 CAR Tregs.

FIGS. 17A-17C. Construction and characterization of anti-CD33 scFv secreting CD4 T-cells. FIG. 17A: Retroviral vector genome: the scFv gene was inserted in frame into an SFG-gamma retroviral vector with a mCherry gene as a fluorescent marker. The T2A peptide sequence was used to link the scFv and mCherry genes. The retroviral control vector carries only the mCherry gene. LTR=long-term repeat, Ψ=psi packaging element, αCD33 scFv=CD33-specific single chain variable fragment, T2A=2A self-cleaving peptide, mCherry=A red fluorescent protein. The CD4 T-cells transduced with retrovirus become mCherry positive (Red cells) and secrete scFv antibody in the supernatant that is detected by Western blot using an anti-T2A secondary antibody. FIG. 17B: Representative histograms show CD4 T cells' secreted scFv binding to CD33 to THP1 and primary human CD14+ cells (Left histograms). The scFv binding to CD33 was diminished in a competition assay where CD33 protein was first blocked with HuM195 antibody (right histograms), reconfirming scFv specificity to CD33 antigen. FIG. 17C: For in-vivo binding studies, control or scFv-secreting CD4 T cells were injected intraperitoneally in mice. Forty-eight hours after T-cell injection, THP1 cell-derived macrophages (THP1-MΦ) were injected into the intraperitoneal cavity. Thirty 30 minutes later, cells were isolated and analyzed for scFv-bound THP1 cells by flow cytometry using an anti-T2A antibody. A clear population of scFv-bound THP1 cells was seen together with decreased total CD33 staining with a commercial antibody, confirming that in vivo binding of the CD4 T cell-secreted scFv to the CD33 that masked the total CD33 staining on THP1-MΦ cells when compared with the control group. Representative image of three independent experiments.

FIGS. 18A-18E. scFv treatment increased Aβ42 uptake by macrophages. FIG. 18A: THP1 cell-derived macrophages (THP1-MΦ) were pre-treated with either PBS (no treatment; NT), purified anti-CD33 scFv, positive control Hum195 (1 ug/ml), control scFv (1 ug/ml), control IgG (1 μg/ml), or Latrunculin as a negative control (1 μM) for 4 hours before pHrodoGreen labeled Aβ42 (1 μM) was added to the cells to initiate phagocytosis. After 48 hours of incubation, cells were analyzed for pHrodoGreen uptake by flow cytometry. pHrodoGreen-Aβ42 uptake by THP1 cells are shown as Mean Fluorescence Intensities (MFI). Like the positive control Hum195, its scFv form also significantly enhanced Aβ42 phagocytosis by THP1-MΦ compared to control scFv or IgG or no antibody-treated (NT) cells. Averages and standard deviations from three independent experiments (n=3) are shown (*p<0.005). FIG. 18B: Primary human CD14+ monocytes were differentiated into macrophages in a 96-well plate using cytokines M-CSF, IL4, and IL13 in culture media. Eight days later, cells were pre-treated as described above in FIG. 18A, and quantification of continuous live imaging of pHrodoRed-Aβ42 uptake by human primary macrophages shown in fluorescent (A.U.) taken every 2 hours for 48 hours. means±SEM; n=4. (***p<0.001). FIG. 18C: Trans well co-culture systems were used to mimic in vivo conditions. Control or scFv secreting CD4 T cells were cultured in the upper well and THP1-MΦ in the lower well for 24 hours before pHrodoGreen-Aβ42 (1 μM) was added into the lower wells to initiate phagocytosis. After 48 hours of incubation, THP1-MΦ were analyzed for pHrodoGreen uptake by fluorescent microscopy and flow cytometry. Lower images show the pHrodoGreen uptake by the THP1-MΦ. Percentage of pHrodoGreen+ THP1-MΦ and their MFI are shown in the upper histograms and bar diagrams respectively. Averages and standard deviations from three independent experiments (n=3) are shown (*p<0.001). FIG. 18D: Pre-treatment of human CD14+ macrophages with anti-CD33 scFv also enhanced their phagocytosis in vivo. Human primary MΦ were pre-treated for 4 hours (as in 2A) before injecting intraperitoneally in mice (5M/mouse) along with pHrodoGreen-Aβ42 (200 ug/mouse). Forty-eight hours late, cells were isolated from the i.p. cavity and analyzed for pHrodoGreen uptake by flow cytometry. Percentage of pHrodoGreen+ macrophages and their MFI are shown in the lower images. Averages and standard deviations from three independent experiments (n=3) are shown (****p<0.001). FIG. 18E: To mimic the actual therapeutic conditions, control or scFv secreting CD4 T cells were injected intraperitoneally in mice and 24 hrs later human primary MΦ D were injected along with pHrodoGreen-Aβ42 (200 ug/mouse). Forty-eight hours later, cells were isolated from the i.p. cavity and analyzed for pHrodoGreen uptake by flow cytometry. Percentage of pHrodoGreen+ macrophages and their MFI are shown in the lower images. Averages and standard deviations from three independent experiments (n=3) are shown (***p<0.001).

FIGS. 19A-19D. Graphical representation of CAR Treg therapy for AP clearance in the AD brain. FIG. 19A: Extracellular deposition of Aβ plaques in the brain leads to an early toxic event in the pathogenesis of AD. Microglia and brain resident macrophages are crucial in clearing the early Aβ fibrils by phagocytosis. However, Aβ fibrils inhibit microglial phagocytosis via engaging with their inhibitory receptor CD33 and other molecules. In addition, Aβ fibril deposition induces the production of pro-inflammatory cytokines by microglia, which contribute to neurodegeneration. FIG. 19B: We have genetically engineered Tregs to express a neuronal antigen-specific CAR and secrete anti-CD33 scFv antibodies. FIG. 19C: These CAR-Tregs would proliferate in response to neuronal antigen in the brain, maintaining an anti-inflammatory environment, and secrete scFv antibody locally. FIG. 19D: CAR Tregs could allow active Aβ phagocytosis by microglia and brain resident macrophages via blocking their inhibitory CD33 receptor via secreted anti-CD33 scFv antibody.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

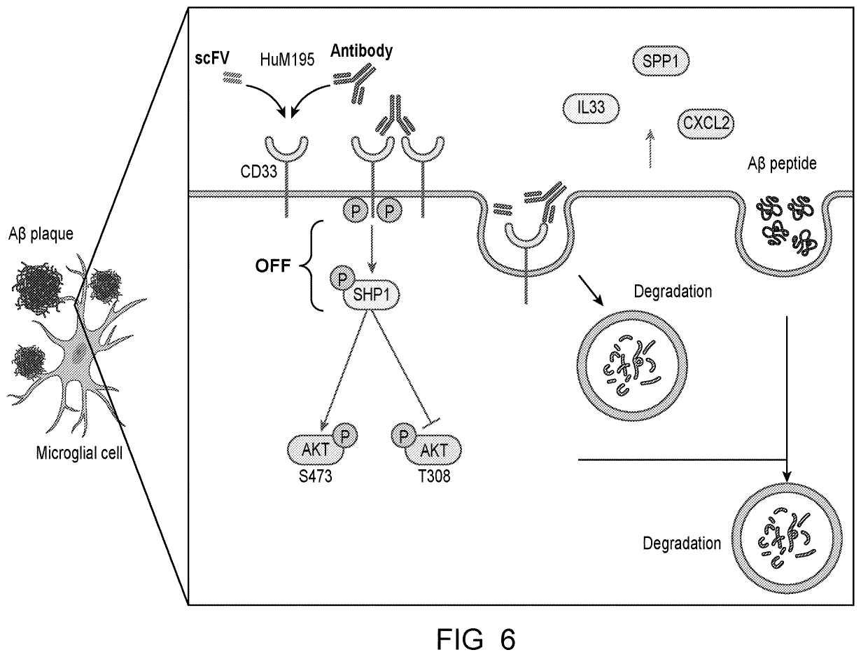

As described herein, immunoglobulin-related compositions comprising lintuzumab (HuM195) sequences are utilized for directly targeting CD33 in AD. Lintuzumab (HuM195) and its scFv activate microglia to enhance Aβ uptake and clearance (FIG. 6). HuM195 antibody or scFv directly binds to CD33 leading to its internalization and degradation, thus activating microglia to increase Aβ clearance capabilities. In addition, the degradation of CD33 modifies the inflammatory response in microglia by increasing expression and secretion of cytokines and chemokines including IL33, CXCL2 and SPP1. In addition, IL33 was shown to stimulate and enhance Aβ uptake. These data demonstrated the efficacy of the immunoglobulin-related compositions of the present technology that directly activate microglia for Aβ clearance and indirectly to facilitate an inflammatory response which may further recruit and amplify the ability to clear the toxic aggregates in AD.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including orally, intranasally, parenterally (intravenously, intramuscularly, intraperitoneally, or subcutaneously), rectally, intrathecally, intratumorally, or topically. Administration includes self-administration and the administration by another.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. As used herein, “antibodies” (includes intact immunoglobulins) and “antigen binding fragments” specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W. H. Freeman & Co., New York, 1997.

More particularly, antibody refers to a polypeptide ligand comprising at least a light chain immunoglobulin variable region or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. Typically, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds CD33 protein will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

“Immunoglobulin-related compositions” as used herein, refers to antibodies (including monoclonal antibodies, polyclonal antibodies, humanized antibodies, chimeric antibodies, recombinant antibodies, multispecific antibodies, bispecific antibodies, etc.,), antibody fragments thereof, and immune cells (e.g., B cells, T cells) that comprise nucleic acids or expression vectors encoding the antibodies and antigen binding fragments. An antibody or antigen binding fragment thereof specifically binds to an antigen.

As used herein, the term “antibody-related polypeptide” means antigen-binding antibody fragments, including single-chain antibodies, that can comprise the variable region(s) alone, or in combination, with all or part of the following polypeptide elements: hinge region, CH₁, CH₂, and CH₃ domains of an antibody molecule. Also included in the technology are any combinations of variable region(s) and hinge region, CH₁, CH₂, and CH₃ domains. Antibody-related molecules useful in the present methods, e.g., but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Examples include: (i) a Fab fragment, a monovalent fragment consisting of the V_{L}, V_{H}, CL and CH₁ domains; (ii) a F(ab′)₂ fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarity determining region (CDR). As such “antibody fragments” or “antigen binding fragments” can comprise a portion of a full length antibody, generally the antigen binding or variable region thereof. Examples of antibody fragments or antigen binding fragments include Fab, Fab′, F(ab′)₂, and Fv fragments; diabodies; linear antibodies; single-chain antibody molecules; and multispecific antibodies formed from antibody fragments.

As used herein, “chimeric antigen receptor” or “CAR”, is a synthetic receptor which grafts or confers a specificity of interest onto an immune effector cell. There are currently three generations of CARs. “First generation” CARs are typically composed of an extracellular antigen binding domain (e.g., a single-chain variable fragment (scFv)), a transmembrane domain, and cytoplasmic/intracellular domain of the T cell receptor (TCR) chain. “First generation” CARs typically have the intracellular domain from the CD3zeta chain, which is the primary transmitter of signals from endogenous TCRs. “First generation” CARs can provide de novo antigen recognition and cause activation of both CD4+ and CD8⁺ T cells through their CD3zeta chain signaling domain in a single fusion molecule, independent of HLA-mediated antigen presentation. In some embodiments, the engineered immune cells provided herein express a “first generation” CAR. “Second generation” CARs add intracellular domains from various co stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (e.g, CD3zeta). In some embodiments, the engineered immune cells provided herein express a “second generation” CAR. “Third generation” CARs comprise those that provide multiple co-stimulation (e.g, CD28 and 4-1BB) and activation (e.g, CD3zeta). In some embodiments, the engineered immune cells provided herein express a “third generation” CAR.

As used herein, the term “chimeric co-stimulatory receptor” or “CCR” refers to a chimeric receptor that binds to an antigen and provides co-stimulatory signals, but does not provide a T-cell activation signal.

As used herein, the term “conjugated” refers to the association of two molecules by any method known to those in the art. Suitable types of associations include chemical bonds and physical bonds. Chemical bonds include, for example, covalent bonds and coordinate bonds. Physical bonds include, for instance, hydrogen bonds, dipolar interactions, van der Waal forces, electrostatic interactions, hydrophobic interactions and aromatic stacking.

As used herein, the term, “co-stimulatory signaling domain,” or “co-stimulatory domain”, refers to the portion of the engineered receptor comprising the intracellular domain of a co-stimulatory molecule. Co-stimulatory molecules are cell surface molecules other than antigen receptors or Fc receptors that provide a second signal required for efficient activation and function of T lymphocytes upon binding to antigen. Examples of such co-stimulatory molecules include CD27, CD28, 4-1BB (CD137), OX40 (CD134), CD30, CD40, PD-1, ICOS (CD278), LFA-1, CD2, CD7, LIGHT, NKD2C, B7-H2 and a ligand that specifically binds CD83. Accordingly, while the present disclosure provides exemplary costimulatory domains derived from CD28 and 4-1BB, other costimulatory domains are contemplated for use with the engineered receptors described herein. The inclusion of one or more co-stimulatory signaling domains can enhance the efficacy and expansion of T cells expressing engineered receptors. The intracellular signaling and co-stimulatory signaling domains can be linked in any order in tandem to the carboxyl terminus of the transmembrane domain.

As used herein, the term “diabodies” refers to small antibody fragments with two antigen-binding sites, which fragments comprise a heavy-chain variable domain (V_{H}) connected to a light-chain variable domain (V_{L}) in the same polypeptide chain (V_{H} V_{L}). By using a linker that is too short to allow pairing between the two domains on the same chain, the domains are forced to pair with the complementary domains of another chain and create two antigen binding sites. Diabodies are described more fully in, e.g., EP 404,097; WO 93/11161; and Hollinger et al., Proc. Natl. Acad. Sci. USA, 90: 6444-6448 (1993).

As used herein, the terms “single-chain antibodies” or “single-chain Fv (scFv)” refer to an antibody fusion molecule of the two domains of the Fv fragment, V_{L} and V_{H}. Single-chain antibody molecules may comprise a polymer with a number of individual molecules, for example, dimer, trimer or other polymers. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single-chain Fv (scFv)). Bird et al. (1988) Science 242:423-426 and Huston et al. (1988) Proc. Natl. Acad Sci. USA 85:5879-5883. Such single-chain antibodies can be prepared by recombinant techniques or enzymatic or chemical cleavage of intact antibodies.

Any of the above-noted antibody fragments are obtained using conventional techniques known to those of skill in the art, and the fragments are screened for binding specificity and neutralization activity in the same manner as are intact antibodies.

As used herein, an “antigen” refers to a molecule to which an antibody (or antigen binding fragment thereof) can selectively bind. The target antigen may be a protein, carbohydrate, nucleic acid, lipid, hapten, or other naturally occurring or synthetic compound. In some embodiments, the target antigen may be a polypeptide (e.g., a CD33 polypeptide). An antigen may also be administered to an animal to generate an immune response in the animal.

The term “antigen binding fragment” refers to a fragment of the whole immunoglobulin structure which possesses a part of a polypeptide responsible for binding to antigen. Examples of the antigen binding fragment useful in the present technology include scFv, (scFv)₂, scFvFc, Fab, Fab′ and F(ab′)₂, but are not limited thereto.

By “binding affinity” is meant the strength of the total noncovalent interactions between a single binding site of a molecule (e.g., an antibody) and its binding partner (e.g., an antigen or antigenic peptide). The affinity of a molecule X for its partner Y can generally be represented by the dissociation constant (K_{D}). Affinity can be measured by standard methods known in the art, including those described herein. A low-affinity complex contains an antibody that generally tends to dissociate readily from the antigen, whereas a high-affinity complex contains an antibody that generally tends to remain bound to the antigen for a longer duration.

As used herein, the term “CDR-grafted antibody” means an antibody in which at least one CDR of an “acceptor” antibody is replaced by a CDR “graft” from a “donor” antibody possessing a desirable antigen specificity.

As used herein, the term “chimeric antibody” means an antibody in which the Fc constant region of a monoclonal antibody from one species (e.g., a mouse Fc constant region) is replaced, using recombinant DNA techniques, with an Fc constant region from an antibody of another species (e.g., a human Fc constant region). See generally, Robinson et al., PCT/US86/02269; Akira et al., European Patent Application 184,187; Taniguchi, European Patent Application 171,496; Morrison et al., European Patent Application 173,494; Neuberger et al., WO 86/01533; Cabilly et al. U.S. Pat. No. 4,816,567; Cabilly et al., European Patent Application 0125,023; Better et al., Science 240: 1041-1043, 1988; Liu et al., Proc. Natl. Acad. Sci. USA 84: 3439-3443, 1987; Liu et al., J. Immunol 139: 3521-3526, 1987; Sun et al., Proc. Natl. Acad. Sci. USA 84: 214-218, 1987; Nishimura et al., Cancer Res 47: 999-1005, 1987; Wood et al., Nature 314: 446-449, 1885; and Shaw et al., J. Natl. Cancer Inst. 80: 1553-1559, 1988.

As used herein, the term “consensus FR” means a framework (FR) antibody region in a consensus immunoglobulin sequence. The FR regions of an antibody do not contact the antigen.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease or condition, a positive control (a compound or composition known to exhibit the desired therapeutic effect) and a negative control (a subject or a sample that does not receive the therapy or receives a placebo) are typically employed.

As used herein, the terms “decrease”, “reduced”, “reduction”, “decrease” or “inhibit” means a decrease by a statistically significant amount. For avoidance of doubt, “decrease”, “reduced”, “reduction”, “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease between 10-100% as compared to a reference level. The terms “decrease”, “reduced”, “reduction”, “decrease” or “inhibit” as used herein in the context of CD33 expression and/or activity means that the expression or activity of CD33 protein or variants or homologues thereof is reduced to an extent, and/or for a time, sufficient to produce the desired effect.

As used herein, the term “effective amount” refers to a quantity of an agent sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “engineered immune cell” refers to an immune cell that is genetically modified.

As used herein, the term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents. In some embodiments, an “epitope” of the CD33 protein is a region of the protein to which the anti-CD33 antibodies of the present technology specifically bind. In some embodiments, the epitope is a conformational epitope or a non-conformational epitope. To screen for anti-CD33 antibodies which bind to an epitope, a routine cross-blocking assay such as that described in Antibodies, A Laboratory Manual, Cold Spring Harbor Laboratory, Ed Harlow and David Lane (1988), can be performed. This assay can be used to determine if an anti-CD33 antibody binds the same site or epitope as an anti-CD33 antibody of the present technology. Alternatively, or additionally, epitope mapping can be performed by methods known in the art. For example, the antibody sequence can be mutagenized such as by alanine scanning, to identify contact residues. In a different method, peptides corresponding to different regions of CD33 protein can be used in competition assays with the test antibodies or with a test antibody and an antibody with a characterized or known epitope.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, “expression control sequence” or “regulatory region” of a nucleic acid molecule means a cis-acting nucleotide sequence that influences expression, positively or negatively, of an operatively linked gene. Regulatory regions include sequences of nucleotides that confer inducible (i.e., require a substance or stimulus for increased transcription) expression of a gene. When an inducer is present or at increased concentration, gene expression can be increased. Regulatory regions also include sequences that confer repression of gene expression (i.e., a substance or stimulus decreases transcription). When a repressor is present or at increased concentration gene expression can be decreased. Regulatory regions are known to influence, modulate or control many in vivo biological activities including cell proliferation, cell growth and death, cell differentiation and immune modulation. Regulatory regions typically bind to one or more trans-acting proteins, which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, but are not limited to, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding site (IRES) elements for the creation of multigene, or polycistronic, messages, polyadenylation signals to provide proper polyadenylation of the transcript of a gene of interest and stop codons, and can be optionally included in an expression vector.

As used herein, the term “gene” means a segment of DNA that contains all the information for the regulated biosynthesis of an RNA product, including promoters, exons, introns, and other untranslated regions that control expression.

As used herein, the terms “increased”, “increase” or “enhance” or “activate” are all used herein to generally mean an increase by a statically significant amount. For the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “heterologous nucleic acid molecule or polypeptide” refers to a nucleic acid molecule (e.g., a cDNA, DNA or RNA molecule) or polypeptide that is either not normally expressed or is expressed at an aberrant level in a cell or sample obtained from a cell. This nucleic acid can be from another organism, or it can be, for example, an mRNA molecule that is not normally expressed in a cell or sample.

As used herein, “humanized” forms of non-human (e.g., murine) antibodies are chimeric antibodies which contain minimal sequence derived from non-human immunoglobulin. For the most part, humanized antibodies are human immunoglobulins in which hypervariable region residues of the recipient are replaced by hypervariable region residues from a non-human species (donor antibody) such as mouse, rat, rabbit or nonhuman primate having the desired specificity, affinity, and capacity. In some embodiments, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance such as binding affinity. Generally, the humanized antibody will comprise substantially all of at least one, and typically two, variable domains (e.g., Fab, Fab′, F(ab′)₂, or Fv), in which all or substantially all of the hypervariable loops correspond to those of a non-human immunoglobulin and all or substantially all of the FR regions are those of a human immunoglobulin consensus FR sequence although the FR regions may include one or more amino acid substitutions that improve binding affinity. The number of these amino acid substitutions in the FR are typically no more than 6 in the H chain, and in the L chain, no more than 3. The humanized antibody optionally may also comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992). See e.g., Ahmed & Cheung, FEBS Letters 588(2):288-297 (2014).

As used herein, the term “hypervariable region” refers to the amino acid residues of an antibody which are responsible for antigen-binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, MD. (1991)) and/or those residues from a “hypervariable loop” (e.g., residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

As used herein, the term “immune cell” refers to any cell that plays a role in the immune response of a subject. Immune cells are of hematopoietic origin, and include lymphocytes, such as B cells and T cells; natural killer cells; myeloid cells, such as monocytes, macrophages, dendritic cells, eosinophils, neutrophils, mast cells, basophils, and granulocytes.

As used herein, the term “intact antibody” or “intact immunoglobulin” means an antibody that has at least two heavy (H) chain polypeptides and two light (L) chain polypeptides interconnected by disulfide bonds. Each heavy chain is comprised of a heavy chain variable region (abbreviated herein as HCVR or V_{H}) and a heavy chain constant region. The heavy chain constant region is comprised of three domains, CH₁, CH₂ and CH₃. Each light chain is comprised of a light chain variable region (abbreviated herein as LCVR or V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR₁, CDR₁, FR₂, CDR₂, FR₃, CDR₃, FR₄. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

As used herein, the terms “individual”, “patient”, or “subject” can be an individual organism, a vertebrate, a mammal, or a human. In some embodiments, the individual, patient or subject is a human.

The term “lymphocyte” refers to all immature, mature, undifferentiated and differentiated white lymphocyte populations including tissue specific and specialized varieties. It encompasses, by way of non-limiting example, B cells, T cells, NKT cells, and NK cells. In some embodiments, lymphocytes include all B cell lineages including pre-B cells, progenitor B cells, early pro-B cells, late pro-B cells, large pre-B cells, small pre-B cells, immature B cells, mature B cells, plasma B cells, memory B cells, B-1 cells, B-2 cells and anergic AN1/T3 cell populations.

The term “monoclonal antibody” as used herein refers to an antibody obtained from a population of substantially homogeneous antibodies, i.e., the individual antibodies comprising the population are identical except for possible naturally occurring mutations that may be present in minor amounts. For example, a monoclonal antibody can be an antibody that is derived from a single clone, including any eukaryotic, prokaryotic, or phage clone, and not the method by which it is produced. A monoclonal antibody composition displays a single binding specificity and affinity for a particular epitope. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibodies can be prepared using a wide variety of techniques known in the art including, e.g., but not limited to, hybridoma, recombinant, and phage display technologies. For example, the monoclonal antibodies to be used in accordance with the present methods may be made by the hybridoma method first described by Kohler et al., Nature 256:495 (1975), or may be made by recombinant DNA methods (See, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

The term “myeloid cell” refers to all immature, mature, undifferentiated, and differentiated white blood cell populations that are derived from myeloid progenitors including tissue specific and specialized varieties, and encompasses, by way of non-limiting example, granulocytes (i.e., mast cells, neutrophils, eosinophils and basophils), monocytes, macrophages, and dendritic cells.

As used herein, “operably linked” with reference to nucleic acid sequences, regions, elements or domains means that the nucleic acid regions are functionally related to each other. For example, nucleic acid encoding a leader peptide can be operably linked to nucleic acid encoding a polypeptide, whereby the nucleic acids can be transcribed and translated to express a functional fusion protein, wherein the leader peptide effects secretion of the fusion polypeptide. In some instances, the nucleic acid encoding a first polypeptide (e.g., a leader peptide) is operably linked to nucleic acid encoding a second polypeptide and the nucleic acids are transcribed as a single mRNA transcript, but translation of the mRNA transcript can result in one of two polypeptides being expressed. For example, an amber stop codon can be located between the nucleic acid encoding the first polypeptide and the nucleic acid encoding the second polypeptide, such that, when introduced into a partial amber suppressor cell, the resulting single mRNA transcript can be translated to produce either a fusion protein containing the first and second polypeptides, or can be translated to produce only the first polypeptide. In another example, a promoter can be operably linked to nucleic acid encoding a polypeptide, whereby the promoter regulates or mediates the transcription of the nucleic acid.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20^{th} edition, ed. A. Gennaro, 2000, Lippincott, Williams & Wilkins, Philadelphia, Pa.).

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the terms “polypeptide,” “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.

As used herein, “prevention”, “prevent”, or “preventing” of a disorder or condition refers to one or more compounds that, in a statistical sample, reduces the occurrence of the disorder or condition in the treated sample relative to an untreated control sample, or delays the onset of one or more symptoms of the disorder or condition relative to the untreated control sample. As used herein, preventing Alzheimer's disease, includes preventing or delaying the initiation of symptoms of Alzheimer's disease. As used herein, prevention of Alzheimer's disease also includes preventing a recurrence of one or more signs or symptoms of Alzheimer's disease.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, the term “sample” refers to clinical samples obtained from a subject. Biological samples may include tissues, cells, protein or membrane extracts of cells, mucus, sputum, bone marrow, bronchial alveolar lavage (BAL), bronchial wash (BW), and biological fluids (e.g., ascites fluid or cerebrospinal fluid (CSF)) isolated from a subject, as well as tissues, cells and fluids (blood, plasma, saliva, urine, serum etc.) present within a subject.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, “specifically binds” refers to a molecule (e.g., an antibody or antigen binding fragment thereof) which recognizes and binds another molecule (e.g., an antigen), but that does not substantially recognize and bind other molecules. The terms “specific binding,” “specifically binds to,” or is “specific for” a particular molecule (e.g., a polypeptide, or an epitope on a polypeptide), as used herein, can be exhibited, for example, by a molecule having a K_{D} for the molecule to which it binds to of about 10⁻⁴ M, 10⁻⁵ M, 10⁻⁶M, 10⁻⁷M, 10⁻⁸ M, 10⁻⁹ M, 10⁻¹⁰ M, 10⁻¹¹ M, or 10⁻¹² M. The term “specifically binds” may also refer to binding where a molecule (e.g., an antibody or antigen binding fragment thereof) binds to a particular polypeptide (e.g., a CD33 polypeptide), or an epitope on a particular polypeptide, without substantially binding to any other polypeptide, or polypeptide epitope.

As used herein, the term “T-cell” includes naive T cells, CD4+ T cells, CD8+ T cells, memory T cells (including central memory T cells, stem-cell-like memory T cells (or stem-like memory T cells), and two types of effector memory T cells: e.g., TEM cells and TEMRA cells), activated T cells, anergic T cells, tolerant T cells, chimeric B cells, Regulatory T cells (also known as suppressor T cells), Natural killer T cells, Mucosal associated invariant T cells, and γΔ T cells, and antigen-specific T cells.

As used herein, “T cell receptor” or “TCR”, is a protein complex found on the surface of T cells, that is responsible for recognizing fragments of antigen as peptides bound to major histocompatibility complex molecules. TCR is composed of two disulfide-linked protein chains. Cells expressing a TCR containing the highly variable alpha (a) and beta (b) chains are referred to as αβ T cells. Cells expressing an alternate TCR, formed by variable gamma (g) and delta (d) chains, are referred to as γΔ T cells. When the TCR engages with antigenic peptide and MHC (peptide/MHC), the T lymphocyte is activated through signal transduction, that is, a series of biochemical events mediated by associated enzymes, co-receptors, specialized adaptor molecules, and activated or released transcription factors. In some embodiments, the TCR is a native T cell receptor that is endogenous to the immune cells. In some embodiments, the TCR is an artificial receptor that mimics native TCR function, i.e., recognizing peptide antigens of key intracellular proteins in the context of MHC on the cell surface.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment or prevention of medical diseases and conditions as described are intended to mean “substantial,” which includes total but also less than total treatment or prevention, and wherein some biologically or medically relevant result is achieved. The treatment may be a continuous prolonged treatment for a chronic disease or a single, or few time administrations for the treatment of an acute condition.

As used herein, a “vector” is a replicable nucleic acid from which one or more heterologous proteins or RNAs can be expressed when the vector is introduced into an appropriate host cell. The vector is used to introduce the nucleic acid encoding the polypeptide or RNA into the host cell for amplification of the nucleic acid or for expression/display of the polypeptide or RNA encoded by the nucleic acid. As used herein, a vector also includes “virus vectors” or “viral vectors.” Viral vectors are engineered viruses that are operatively linked to exogenous genes to transfer (as vehicles or shuttles) the exogenous genes into cells.

### Alzheimer's Disease

Alzheimer's Disease Pathogenesis. Alzheimer's disease (AD) is a complicated, multifactorial, progressive neurodegenerative disorder of the brain, which is characterized by memory deterioration, behavioral disturbances, impairment of activities of daily living, and loss of independent function. AD is the most common form of dementia and although its prevalence is much higher in the older population, it is still the most frequent form of dementia under the age of 65 years. AD can be further categorized based on the age of onset: late onset AD (i.e., onset at >65 years); early onset AD (i.e., onset at <50 years); and intermediate onset AD (i.e., onset between 50-65 years). In all categories of the disease, the pathology is the same but the Aβ abnormalities tend to be more severe and widespread in cases beginning at an earlier age. AD is characterized at the macroscopic level by significant brain shrinkage away from the cranial vault as seen in MRI images as a direct result of neuronal loss and by two types of macroscopic lesions in the brain, senile amyloid plaques and neurofibrillary tau tangles. Senile plaques are areas comprising disorganized neuronal processes up to 150 μm across and extracellular amyloid deposits, which are typically concentrated at the center and visible by microscopic analysis of sections of brain tissue. Neurofibrillary tangles are intracellular deposits of tau protein containing two filaments twisted about each other in pairs.

The principal constituent of the plaques is a peptide termed Aβ or β-amyloid peptide. Aβ peptide is an internal fragment of 39-43 amino acids of a precursor protein termed amyloid precursor protein (APP). Several mutations within the APP protein have been correlated with the presence of Alzheimer's disease. See, e.g., Goate et al, Nature 349: 704 (1991) (valine⁷¹⁷ to isoleucine); Chartier Harlan et al. Nature 353: 844 (1991) (valine⁷¹⁷ to glycine); Murrell et al, Science 254: 97 (1991) (valine⁷¹⁷ to phenylalanine); Mullan et al, Nature Genet. 1: 345 (1992) (a double mutation changing lysine⁵⁹⁵-methionine⁵⁹⁶ to asparagine⁵⁹⁵-leucine⁵⁹⁶). Such mutations are believed to cause Alzheimer's disease by increased or altered processing of APP to Aβ, particularly processing of APP to increased amounts of the long form of Aβ (i.e., Aβ 1-42 and Aβ 1-43). Mutations in other genes, such as the presenilin genes, PS1 and PS2, are thought indirectly to affect processing of APP to generate increased amounts of long form Aβ (Hardy, TINS 20: 154 (1997)). These observations indicate that Aβ, and particularly its long form, is a causative element in Alzheimer's disease.

Aβ is generated by processing of APP protein by two enzymes, termed β and γ secretases. Known mutations in APP associated with Alzheimer's disease occur proximate to the site of β or γ-secretase, or within Aβ. For example, position 717 is proximate to the site of γ-secretase cleavage of APP in its processing to Aβ, and positions 670/671 are proximate to the site of β-secretase cleavage. It is believed that the mutations cause AD disease by interacting with the cleavage reactions by which Aβ is formed so as to increase the amount of the 42/43 amino acid form of Aβ generated.

Aβ has the unusual property in that it can fix and activate both classical and alternate complement cascades. In particular, Aβ binds to C1q and ultimately to C3bi, which facilitates binding to macrophages leading to activation of B cells. In addition, C3bi breaks down further and then binds to CR2 on B cells in a T cell dependent manner leading to a 10,000 increase in activation of these cells. This mechanism causes Aβ to generate an immune response in excess of that of other antigens.

Most therapeutic strategies for Alzheimer's disease are aimed at reducing or eliminating the deposition of Aβ42 in the brain, typically via reduction in the generation of Aβ42 from APP and/or some means of lowering existing Aβ42 levels from sources that directly contribute to the deposition of Aβ peptide in the brain. A partial list of aging-associated causative factors in the development of sporadic Alzheimer's disease includes a shift in the balance between Aβ peptide production and its clearance from neurons that favors intracellular accumulation, increased secretion of Aβ peptides by neurons into the surrounding extracellular space, increased levels of oxidative damage to these cells, and global brain hypoperfusion and the associated compensatory metabolic shifts in affected tissue.

The Aβ42 deposits within neurons and plaques could also originate from outside of the neurons (exogenous Aβ42) during Alzheimer's disease pathogenesis. Levels of soluble Aβ peptides in the blood are known to be much higher than the interstitial space and CSF in the brains of healthy individuals with blood as a source of exogenous Aβ peptides that eventually deposit in the Alzheimer's disease brain. However, except for trace amounts of Aβ that are actively transported across endothelial cells, it is well-known that access of blood-borne Aβ peptides to brain tissue in normal healthy individuals is effectively blocked by the integrity of the blood-brain barrier (BBB).

Symptoms of AD include, but are not limited to, cognitive dysfunction or decline; memory loss; agitation; mood swings; impaired judgment; dementia; difficulty with abstract thinking; difficulty with familiar tasks; disorientation; diminished communication skills; repetitive speech or actions; impaired visuospatial abilities; impaired speaking, reading, and writing; withdrawal; depression; loss of recognition; loss of motor skills and sense of touch; delusions; paranoia; verbal or physical aggression; and sleep disorders.

Subjects at risk for or predisposed to the development of AD can be identified by, e.g., any one or a combination of diagnostic or prognostic assays known in the art. Although advanced age is the greatest known risk factor for AD, the disease is multifactorial and a number of other risk factors have been identified, including family history. First-degree relatives of patients with AD are more likely to develop the disease. Genetic markers of risk toward Alzheimer's disease include mutations in the APP gene, the presenilin genes (PS1 and PS2), and APOE4. In addition, mutations in CD33, CLU, BIN1, PICALM, CR1, CD2AP, EPHA1, ABCA7, MS4A4A/MS4A6E and TREM2, may increase the likelihood of developing the disease.

Methods for identifying subjects at risk for or suffering from Alzheimer's Disease. Subjects amenable to the therapeutic and/or prophylactic methods disclosed herein include subjects that are at risk for, or are diagnosed with Alzheimer's Disease. Subjects may be screened for their likelihood of developing Alzheimer's Disease or diagnosed with Alzheimer's Disease based on a number of biochemical and genetic markers.

Genetic abnormality in a few families has been traced to chromosome 1 (St. George-Hyslop et al, Science 235: 885-890 (1987)). One genetic marker includes mutations in the APP gene, particularly mutations at position 717 and positions 670 and 671, referred to as the Hardy and Swedish mutations respectively. Other markers of risk are mutations in the presenilin genes (PS1 and PS2), and APOE4, family history of Alzheimer's Disease, hypercholesterolemia or atherosclerosis. Subjects with APP, PS1 or PS2 mutations are highly likely to develop Alzheimer's disease. APOE is a susceptibility gene, and subjects with the APOE4 isoform have an increased risk of developing Alzheimer's disease. Test for subjects with APOE4 isoform are disclosed in U.S. Pat. No. 6,027,896, which is incorporated in its entirety herein by reference. Other genetic links have been associated with an increased risk of Alzheimer's disease, for example variances in the neuronal sortilin-related receptor SORL1, may have increased likelihood of developing late-onset Alzheimer's Disease. Other potential Alzheimer Disease susceptibility genes, include, for example ACE, CHRNB2, CST3, ESR1, GAPDHS, IDE, MTHFR, NCSTN, PRNP, PSEN1, TF, TFAM and TNF may be used to identify subjects with increased risk of developing Alzheimer's Disease, as well as variances in the alpha-T catenin (VR22) gene. As disclosed herein, CD33 protein or gene encoding the same is also associated with Alzheimer's Disease.

One may also diagnose a subject with increased risk of developing Alzheimer's Disease on the basis of a simple eye test, where the presence of cataracts and/or Aβ in the lens identifies a subject with increased risk of developing Alzheimer's disease. Methods to detect Alzheimer's Disease include using a quasi-elastic light scattering device from Neuroptix, using Quasi-Elastic Light Scattering (QLS) and Fluorescent Ligand Scanning (FLS) and a Neuroptix™ QEL scanning device, to enable non-invasive quantitative measurements of amyloid aggregates in the eye, to examine and measure deposits in specific areas of the lens as an early diagnostic for Alzheimer's disease. Methods to diagnose a subject at risk of developing Alzheimer's Disease using such a method of non-invasive eye test are disclosed in U.S. Pat. No. 7,107,092, which is incorporated in its entirety herein by reference.

Individuals presently suffering from Alzheimer's Disease can be recognized from characteristic dementia, as well as the presence of risk factors described above. In addition, a number of diagnostic tests are available for identifying individuals who have AD. These include measurement of CSF tau and Aβ42 levels. Elevated tau and decreased Aβ42 levels signify the presence of Alzheimer's Disease.

There are two alternative “criteria” which are utilized to clinically diagnose Alzheimer's Disease: the DSM-IIIR criteria and the NINCDS-ADRDA criteria (which is an acronym for National Institute of Neurological and Communicative Disorders and Stroke (NINCDS) and the Alzheimer's Disease and Related Disorders Association (ADRDA). Briefly, the criteria for diagnosis of Alzheimer's Disease under DSM-IIIR include (1) dementia, (2) insidious onset with a generally progressive deteriorating course, and (3) exclusion of all other specific causes of dementia by history, physical examination, and laboratory tests. Within the context of the DSM-IIIR criteria, dementia is understood to involve a multifaceted loss of intellectual abilities, such as memory, judgement, abstract thought, and other higher cortical functions, and changes in personality and behaviour.

In contrast, the NINCDS-ADRDA criteria sets forth three categories of Alzheimer's Disease, including “probable,” “possible,” and “definite” Alzheimer's Disease. Clinical diagnosis of “possible” Alzheimer's Disease may be made on the basis of a dementia syndrome, in the absence of other neurologic, psychiatric or systemic disorders sufficient to cause dementia. Criteria for the clinical diagnosis of “probable” Alzheimer's Disease include (a) dementia established by clinical examination and documented by a test such as the Mini-Mental test (b) deficits in two or more areas of cognition; (c) progressive worsening of memory and other cognitive functions; (d) no disturbance of consciousness; (e) onset between ages 40 and 90, most often after age 65; and (f) absence of systemic orders or other brain diseases that could account for the dementia. The criteria for definite diagnosis of Alzheimer's Disease include histopathologic evidence obtained from a biopsy, or after autopsy. Since confirmation of definite Alzheimer's Disease requires histological examination from a brain biopsy specimen (which is often difficult to obtain), it is rarely used for early diagnosis of Alzheimer's Disease.

One can also use neuropathologic diagnosis of Alzheimer's Disease, where the numbers of plaques and tangles in the neurocortex (frontal, temporal, and parietal lobes), hippocampus and amygdala are analyzed. Alternatively, quantitative electroencephalographic analysis (EEG) may be used to diagnose Alzheimer's Disease. This method employs Fourier analysis of the beta, alpha, theta, and delta bands for diagnosis of Alzheimer's Disease.

One can also diagnose Alzheimer's Disease by quantifying the degree of neural atrophy, since such atrophy is generally accepted as a consequence of Alzheimer's Disease. Examples of these methods include computed tomographic scanning (CT), and magnetic resonance imaging (MRI). Alzheimer's Disease may be diagnosed by assessing decreased cerebral blood flow or metabolism in the posterior temporoparietal cerebral cortex by measuring decreased blood flow or metabolism by positron emission tomography (PET), single photon emission computed tomography (SPECT), and xenon inhalation methods.

One can also immunologically diagnose Alzheimer's. Wolozin and coworkers (Wolozin et al, Science 232:648-650, 1986) produced a monoclonal antibody “Alz50,” that reacts with a 68-kDa protein “A68,” which is expressed in the plaques and neuron tangles of patients with Alzheimer's Disease. Using the antibody Alz50 and Western blot analysis, A68 was detected in the cerebral spinal fluid (CSF) of some Alzheimer's patients and not in the CSF of normal elderly patients.

One can also diagnose Alzheimer's Disease using neurochemical markers of Alzheimer's disease. Neurochemical markers which have been associated with Alzheimer's Disease include reduced levels of acetylcholinesterase, reduced somatostatin, a negative relation between serotonin and 5-hydroxyindoleacetic acid, greater probenecid-induced rise in homovanyllic acid and reduced neuron-specific enolase. Other methods to diagnose a patient at risk of or having a neurodegenerative disease or disorder, such as Alzheimer's Disease includes measurement of CD33 activity and/or expression using the methods as disclosed herein, for example using quantitative RT-PCR.

Although a definitive diagnosis of AD can only be made in a post-mortem neuropathologic evaluation (through the detection of extracellular amyloid plaques and intracellular neurofibrillary tangles in brain tissue), clinical Alzheimer's disease in humans may be diagnosed by a combination of symptoms and the results of certain tests to rule out other conditions before making a diagnosis. Medical evaluations typically include patient history, physical examination, and neuropsychological testing. To diagnose a subject as having AD, tests involving memory, problem solving, attention, counting, language, balance, senses, and reflexes may be conducted. Standard medical tests, such as blood and urine tests, or brain scans may be conducted to rule out other possible causes of the symptoms. To confirm a diagnosis of AD, the following must be present and severe enough to affect daily activities: gradual memory loss and progressing cognitive impairment. In some cases, genetic testing may be appropriate. For example, the APOE4 risk allele is associated with higher likelihood of individuals over the age of 55 years developing AD and could serve as a predictor of developing the disease. Additional emerging tests may also enable the assessment of biomarkers in people who may be at risk of AD. Without wishing to be bound by theory, it is believed that promoting the clearance of the toxic Aβ peptides can prevent development of the disease. Microglia, brain resident macrophage cells, are responsible for removal of debris and toxic material in the brain. These cells can uptake Aβ plaques and tau effectively and remove them from the brain, thereby preventing cognitive decline.

### CD33

CD33, a sialic acid binding transmembrane receptor, is expressed on the surface of cells of myeloid lineage such as microglia. Sialic acid appears mainly in animal tissues and is added in the last step of glycosylation process with high concentration in the brain. Upon sialic acid binding, the immunoreceptor tyrosine-based inhibition motif (ITIM) of CD33 in its cytosolic portion is phosphorylated and acts as a docking site for Src homology 2 (SH2) domain-containing proteins like SHP phosphatases. This results in a signaling cascade that inhibits phagocytosis in the cell. Recent genetic studies suggest a correlation between CD33 to AD (Ries M., Sastre M., Front Aging Neurosci 8: 160 (2016)). CD33 expression is elevated in the brains of AD patients, which presumably inhibits microglia activation and clearing toxic proteins (Jiang T., et al., Mol Neurobiol 49: 529-535 (2014)).

Because of the expression of CD33 in myeloid cells, antibodies against CD33 are available and had been developed for Acute Myeloid Leukemia (AML), specifically Lintuzumab (Hu195) (Sutherland M. K., et al., MAbs. 1: 481-490 (2009)).

CD33 inhibitory antibodies face major challenges as a treatment for AD. First, the physical obstacle of the blood-brain-barrier (BBB) prevents effective CNS drug delivery (13). The current notion states that only 0.1% of circulating antibodies or therapeutic macromolecules cross the BBB (43).

The immunoglobulin-related compositions disclosed herein are useful for treating AD by neutralizing CD33 in microglia cells and eliminating the inhibitory effect of CD33, hence increasing the phagocytic ability for Aβ clearance.

### Anti-CD33 Immunoglobulin-Related Compositions of the Present Technology

The present technology describes compositions for the use of anti-CD33 immunoglobulin-related compositions (e.g., anti-CD33 antibodies or antigen binding fragments thereof) for the treatment of Alzheimer's disease. Anti-CD33 immunoglobulin-related compositions within the scope of the present technology are derived from Lintuzumab (Hul95) and include, e.g., but are not limited to, monoclonal, chimeric, humanized, bispecific antibodies and diabodies that specifically bind the target polypeptide, a homolog, derivative or a fragment thereof. The present disclosure also provides antigen binding fragments of any of the anti-CD33 antibodies disclosed herein, wherein the antigen binding fragment is selected from the group consisting of Fab, F(ab)′2, Fab′, scFv, and Fv.

In one aspect, the present technology provides an antibody or antigen binding fragment thereof comprising a heavy chain immunoglobulin variable domain (V_{H}) and a light chain immunoglobulin variable domain (V_{L}), wherein (a) the V_{H} comprises an amino acid sequence of: SEQ ID NO: 1; and/or (b) the V_{L} comprises an amino acid sequence of: SEQ ID NO: 2.

In any of the above embodiments, the antibody further comprises a Fc domain of any isotype, e.g., but are not limited to, IgG (including IgG1, IgG2, IgG3, and IgG4), IgA (including IgA₁ and IgA₂), IgD, IgE, or IgM, and IgY. Non-limiting examples of constant region sequences include:

Human IgD constant region, Uniprot: P01880
(SEQ ID NO: 5)
APTKAPDVFPIISGCRHPKDNSPVVLACLITGYHPTSVTVTWYMGTQSQP
QRTFPEIQRRDSYYMTSSQLSTPLQQWRQGEYKCVVQHTASKSKKEIFRW
PESPKAQASSVPTAQPQAEGSLAKATTAPATTRNTGRGGEEKKKEKEKEE
QEERETKTPECPSHTQPLGVYLLTPAVQDLWLRDKATFTCFVVGSDLKDA
HLTWEVAGKVPTGGVEEGLLERHSNGSQSQHSRLTLPRSLWNAGTSVTCT
LNHPSLPPQRLMALREPAAQAPVKLSLNLLASSDPPEAASWLLCEVSGFS
PPNILLMWLEDQREVNTSGFAPARPPPQPGSTTFWAWSVLRVPAPPSPQP
ATYTCVVSHEDSRTLLNASRSLEVSYVTDHGPMK
Human IgG1 constant region, Uniprot: P01857
(SEQ ID NO: 6)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
Human IgG2 constant region, Uniprot: P01859
(SEQ ID NO: 7)
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDISVEWESNGQPENNYKTTPPMLDSDGSFFLYSKLTVDKSRWQQGN
VFSCSVMHEALHNHYTQKSLSLSPGK
Human IgG3 constant region, Uniprot: P01860
(SEQ ID NO: 8)
ASTKGPSVFPLAPCSRSTSGGTAALGCLVKDYFPEPVTVSWNSGALTSGV
HTFPAVLQSSGLYSLSSVVTVPSSSLGTQTYTCNVNHKPSNTKVDKRVEL
KTPLGDTTHTCPRCPEPKSCDTPPPCPRCPEPKSCDTPPPCPRCPEPKSC
DTPPPCPRCPAPELLGGPSVFLFPPKPKDTLMISRTPEVTCVVVDVSHED
PEVQFKWYVDGVEVHNAKTKPREEQYNSTFRVVSVLTVLHQDWLNGKEYK
CKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQG
NIFSCSVMHEALHNRFTQKSLSLSPGK
Human IgM constant region, Uniprot: P01871
(SEQ ID NO: 9)
GSASAPTLFPLVSCENSPSDTSSVAVGCLAQDFLPDSITLSWKYKNNSDI
SSTRGFPSVLRGGKYAATSQVLLPSKDVMQGTDEHVVCKVQHPNGNKEKN
VPLPVIAELPPKVSVFVPPRDGFFGNPRKSKLICQATGFSPRQIQVSWLR
EGKQVGSGVTTDQVQAEAKESGPTTYKVTSTLTIKESDWLGQSMFTCRVD
HRGLTFQQNASSMCVPDQDTAIRVFAIPPSFASIFLTKSTKLTCLVTDLT
TYDSVTISWTRQNGEAVKTHTNISESHPNATFSAVGEASICEDDWNSGER
FTCTVTHTDLPSPLKQTISRPKGVALHRPDVYLLPPAREQLNLRESATIT
CLVTGFSPADVFVQWMQRGQPLSPEKYVTSAPMPEPQAPGRYFAHSILTV
SEEEWNTGETYTCVAHEALPNRVTERTVDKSTGKPTLYNVSLVMSDTAGT
CY
Human IgG4 constant region, Uniprot: P01861
(SEQ ID NO: 10)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK
Human IgA1 constant region, Uniprot: P01876
(SEQ ID NO: 11)
ASPTSPKVFPLSLCSTQPDGNVVIACLVQGFFPQEPLSVTWSESGQGVTA
RNFPPSQDASGDLYTTSSQLTLPATQCLAGKSVTCHVKHYTNPSQDVTVP
CPVPSTPPTPSPSTPPTPSPSCCHPRLSLHRPALEDLLLGSEANLTCTLT
GLRDASGVTFTWTPSSGKSAVQGPPERDLCGCYSVSSVLPGCAEPWNHGK
TFTCTAAYPESKTPLTATLSKSGNTFRPEVHLLPPPSEELALNELVTLTC
LARGFSPKDVLVRWLQGSQELPREKYLTWASRQEPSQGTTTFAVTSILRV
AAEDWKKGDTFSCMVGHEALPLAFTQKTIDRLAGKPTHVNVSVVMAEVDG
TCY
Human IgA2 constant region, Uniprot: P01877
(SEQ ID NO: 12)
ASPTSPKVFPLSLDSTPQDGNVVVACLVQGFFPQEPLSVTWSESGQNVTA
RNFPPSQDASGDLYTTSSQLTLPATQCPDGKSVTCHVKHYTNPSQDVTVP
CPVPPPPPCCHPRLSLHRPALEDLLLGSEANLTCTLTGLRDASGATFTWT
PSSGKSAVQGPPERDLCGCYSVSSVLPGCAQPWNHGETFTCTAAHPELKT
PLTANITKSGNTFRPEVHLLPPPSEELALNELVTLTCLARGFSPKDVLVR
WLQGSQELPREKYLTWASRQEPSQGTTTFAVTSILRVAAEDWKKGDTFSC
MVGHEALPLAFTQKTIDRMAGKPTHVNVSVVMAEVDGTCY
Human Ig kappa constant region, Uniprot: P01834
(SEQ ID NO: 13)
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC

In some embodiments, the immunoglobulin-related compositions of the present technology comprise a heavy chain constant region that is at least 80%, at least 85%, at least 90%, at least 95%, at least 99%, or is 100% identical to SEQ ID NOS: 5-12. Additionally or alternatively, in some embodiments, the immunoglobulin-related compositions of the present technology comprise a light chain constant region that is at least 80%, at least 85%, at least 90%, at least 95%, at least 99%, or is 100% identical to SEQ ID NO: 13. In some embodiments, the immunoglobulin-related compositions of the present technology bind to the extracellular domain of CD33. In some embodiments, the epitope is a conformational epitope or a non-conformational epitope.

Additionally or alternatively, in some embodiments, the Fc domain comprises a blood-brain barrier (BBB) target epitope. In certain embodiments, the BBB target epitope comprises an IgG constant region comprising a plurality of amino acid substitutions selected from the group consisting of: (a) N384L, Q386L, P387V, E388W, N389V, N390G, D413A, R416T, and N421W; (b) N384Y, Q386T, P387V, E388W, N389S, N390H, D413S, R416E, and N421Y; (c) N384Y, Q386T, P387E, E388W, N389S, N390Q, D413E, R416D, and N421H; (d) N384V, Q386T, P387P, E388W, N389A, N390L, D413L, R416E, and N421W; (e) N384L, Q386H, P387V, E388W, N389A, N390V, D413P, R416T, and N421W; (f) N384Y, Q386T, P387E, E388W, N389S, N390S, D413T, R416E, and N421F; (g) N384Y, Q386T, P387E, E388W, N389S, N390S, D413T, S415E, R416E, and N421F; (h) E380W, N384Y, Q386T, P387E, E388W, N389S, N390S, D413T, S415E, R416E, and N421F; (i) E380W, N384Y, Q386T, P387E, E388W, N389S, N390S, K392R, D413T, K414R, S415E, R416E, N421F, S424T and S426G; and (j) E380L, N384Y, Q386T, P387E, E388W, N389S, N390S, D413T, S415E, R416E, and N421F.

In any of the above embodiments of the immunoglobulin-related compositions, the heavy chain and light chain immunoglobulin variable domain sequences form an antigen binding site that binds to the extracellular domain of CD33. In some embodiments, the epitope is a conformational epitope or a non-conformational epitope.

In some embodiments, the heavy chain and light chain immunoglobulin variable domain sequences are components of the same polypeptide chain. In other embodiments, the heavy chain and light chain immunoglobulin variable domain sequences are components of different polypeptide chains. In certain embodiments, the antibody is a full-length antibody.

In some embodiments, the immunoglobulin-related compositions of the present technology bind specifically to at least one CD33 polypeptide. In some embodiments, the immunoglobulin-related compositions of the present technology bind at least one CD33 polypeptide with a dissociation constant (K_{D}) of about 10⁻³M, 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. In certain embodiments, the immunoglobulin-related compositions are monoclonal antibodies, chimeric antibodies, humanized antibodies, or bispecific antibodies. In some embodiments, the antibodies comprise a human antibody framework region.

In certain embodiments, the immunoglobulin-related composition includes one or more of the following characteristics: (a) a light chain immunoglobulin variable domain sequence that is at least 80%, at least 85%, at least 90%, at least 95%, or at least 99% identical to the light chain immunoglobulin variable domain sequence present in any one of SEQ ID NO: 2; and/or (b) a heavy chain immunoglobulin variable domain sequence that is at least 80%, at least 85%, at least 90%, at least 95%, or at least 99% identical to the heavy chain immunoglobulin variable domain sequence present in any one of SEQ ID NO: 1. In another aspect, one or more amino acid residues in the immunoglobulin-related compositions provided herein are substituted with another amino acid. The substitution may be a “conservative substitution” as defined below:

| Original | Exemplary | Conservative |
|---|---|---|
| Residue | Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

In one aspect, the present disclosure provides an immunoglobulin-related composition comprising an amino acid sequence that is at least 80%, at least 85%, at least 90%, at least 95%, or at least 99% identical to the amino acid sequence of SEQ TD NO: 3. In certain embodiments, an immunoglobulin-related composition of the present disclosure comprises the amino acid sequence of SEQ TD NO: 3.

In certain embodiments, the immunoglobulin-related compositions contain an IgG1 constant region comprising one or more amino acid substitutions selected from the group consisting of N297A and K322A. Additionality or alternatively, in some embodiments, the immunoglobulin-related compositions contain an IgG4 constant region comprising a S228P mutation.

In some aspects, the anti-CD33 immunoglobulin-related compositions described herein contain structural modifications to facilitate rapid binding and cell uptake and/or slow release. In some aspects, the anti-CD33 immunoglobulin-related composition of the present technology (e.g., an antibody) may contain a deletion in the CH₂ constant heavy chain region to facilitate rapid binding and cell uptake and/or slow release. In some aspects, a Fab fragment is used to facilitate rapid binding and cell uptake and/or slow release. In some aspects, a F(ab)′₂ fragment is used to facilitate rapid binding and cell uptake and/or slow release.

In one aspect, the present technology provides recombinant nucleic acid sequences encoding any of the immunoglobulin-related compositions described herein. In some embodiments, the recombinant nucleic acid sequence is SEQ ID NO: 4.

In another aspect, the present technology provides a host cell expressing any nucleic acid sequence encoding any of the immunoglobulin-related compositions described herein.

The immunoglobulin-related compositions of the present technology (e.g., an anti-CD33 antibody) can be monospecific, bispecific, trispecific or of greater multispecificity. Multispecific antibodies can be specific for different epitopes of one or more CD33 polypeptides or can be specific for both the CD33 polypeptide(s) as well as for heterologous compositions, such as a heterologous polypeptide or solid support material. See, e.g., WO 93/17715; WO 92/08802; WO 91/00360; WO 92/05793; Tutt et al., J. Immunol. 147: 60-69 (1991); U.S. Pat. Nos. 5,573,920, 4,474,893, 5,601,819, 4,714,681, 4,925,648; 6,106,835; Kostelny et al., J. Immunol. 148: 1547-1553 (1992). In some embodiments, the immunoglobulin-related compositions are chimeric. In certain embodiments, the immunoglobulin-related compositions are humanized.

The immunoglobulin-related compositions of the present technology can further be recombinantly fused to a heterologous polypeptide at the N- or C-terminus or chemically conjugated (including covalently and non-covalently conjugations) to polypeptides or other compositions. For example, the immunoglobulin-related compositions of the present technology can be recombinantly fused or conjugated to molecules useful as labels in detection assays and effector molecules such as heterologous polypeptides, drugs, or toxins. See, e.g., WO 92/08495; WO 91/14438; WO 89/12624; U.S. Pat. No. 5,314,995; and EP 0 396 387.

In any of the above embodiments of the immunoglobulin-related compositions of the present technology, the antibody or antigen binding fragment may be optionally conjugated to an agent selected from the group consisting of isotopes, dyes, chromagens, contrast agents, drugs, toxins, cytokines, enzymes, enzyme inhibitors, hormones, hormone antagonists, growth factors, radionuclides, metals, liposomes, nanoparticles, RNA, DNA or any combination thereof. For a chemical bond or physical bond, a functional group on the immunoglobulin-related composition typically associates with a functional group on the agent. Alternatively, a functional group on the agent associates with a functional group on the immunoglobulin-related composition.

The functional groups on the agent and immunoglobulin-related composition can associate directly. For example, a functional group (e.g., a sulfhydryl group) on an agent can associate with a functional group (e.g., sulfhydryl group) on an immunoglobulin-related composition to form a disulfide. Alternatively, the functional groups can associate through a cross-linking agent (i.e., linker). Some examples of cross-linking agents are described below. The cross-linker can be attached to either the agent or the immunoglobulin-related composition. The number of agents or immunoglobulin-related compositions in a conjugate is also limited by the number of functional groups present on the other. For example, the maximum number of agents associated with a conjugate depends on the number of functional groups present on the immunoglobulin-related composition. Alternatively, the maximum number of immunoglobulin-related compositions associated with an agent depends on the number of functional groups present on the agent.

In yet another embodiment, the conjugate comprises one immunoglobulin-related composition associated to one agent. In one embodiment, a conjugate comprises at least one agent chemically bonded (e.g., conjugated) to at least one immunoglobulin-related composition. The agent can be chemically bonded to an immunoglobulin-related composition by any method known to those in the art. For example, a functional group on the agent may be directly attached to a functional group on the immunoglobulin-related composition. Some examples of suitable functional groups include, for example, amino, carboxyl, sulfhydryl, maleimide, isocyanate, isothiocyanate and hydroxyl.

The agent may also be chemically bonded to the immunoglobulin-related composition by means of cross-linking agents, such as dialdehydes, carbodiimides, dimaleimides, and the like. Cross-linking agents can, for example, be obtained from Pierce Biotechnology, Inc., Rockford, Ill. The Pierce Biotechnology, Inc. web-site can provide assistance. Additional cross-linking agents include the platinum cross-linking agents described in U.S. Pat. Nos. 5,580,990; 5,985,566; and 6,133,038 of Kreatech Biotechnology, B.V., Amsterdam, The Netherlands.

Alternatively, the functional group on the agent and immunoglobulin-related composition can be the same. Homobifunctional cross-linkers are typically used to cross-link identical functional groups. Examples of homobifunctional cross-linkers include EGS (i.e., ethylene glycol bis[succinimidylsuccinate]), DSS (i.e., disuccinimidyl suberate), DMA (i.e., dimethyl adipimidate.2HCl), DTSSP (i.e., 3,3′-dithiobis[sulfosuccinimidylpropionate])), DPDPB (i.e., 1,4-di-[3′-(2′-pyridyldithio)-propionamido]butane), and BMH (i.e., bis-maleimidohexane). Such homobifunctional cross-linkers are also available from Pierce Biotechnology, Inc.

In other instances, it may be beneficial to cleave the agent from the immunoglobulin-related composition. The web-site of Pierce Biotechnology, Inc. described above can also provide assistance to one skilled in the art in choosing suitable cross-linkers which can be cleaved by, for example, enzymes in the cell. Thus the agent can be separated from the immunoglobulin-related composition. Examples of cleavable linkers include SMPT (i.e., 4-succinimidyloxycarbonyl-methyl-a-[2-pyridyldithio]toluene), Sulfo-LC-SPDP (i.e., sulfosuccinimidyl 6-(3-[2-pyridyldithio]-propionamido)hexanoate), LC-SPDP (i.e., succinimidyl 6-(3-[2-pyridyldithio]-propionamido)hexanoate), Sulfo-LC-SPDP (i.e., sulfosuccinimidyl 6-(3-[2-pyridyldithio]-propionamido)hexanoate), SPDP (i.e., N-succinimidyl 3-[2-pyridyldithio]-propionamidohexanoate), and AEDP (i.e., 3-[(2-aminoethyl)dithio]propionic acid HCl).

In another embodiment, a conjugate comprises at least one agent physically bonded with at least one immunoglobulin-related composition. Any method known to those in the art can be employed to physically bond the agents with the immunoglobulin-related compositions. For example, the immunoglobulin-related compositions and agents can be mixed together by any method known to those in the art. The order of mixing is not important. For instance, agents can be physically mixed with immunoglobulin-related compositions by any method known to those in the art. For example, the immunoglobulin-related compositions and agents can be placed in a container and agitated, by for example, shaking the container, to mix the immunoglobulin-related compositions and agents.

The immunoglobulin-related compositions can be modified by any method known to those in the art. For instance, the immunoglobulin-related composition may be modified by means of cross-linking agents or functional groups, as described above.

Additionally or alternately, in some embodiments, the immunoglobulin-related compositions of the present technology are conjugated to insulin, transferrin, an interleukin, albumin, a plasma protein, a lipoprotein, a RVG29 peptide, a Mini-Aβ4 (apamin) peptide, a shark antibody, an antibody that targets insulin receptor, an antibody that targets interleukin receptor, or an antibody that targets transferrin receptor. In certain embodiments, the immunoglobulin-related compositions of the present technology are encapsulated by a nanoparticle or an exosome.

### Engineered Immune Cells

The presently disclosed subject matter provides engineered immune cells that express and secrete any and all embodiments of the anti-CD33 antibodies or antigen binding fragments described herein.

In one aspect, the present disclosure provides an engineered immune cell comprising: (a) a neuronal antigen-specific receptor and/or a nucleic acid encoding the neuronal antigen-specific receptor; and (b) an anti-CD33 antibody, or an antigen binding fragment thereof and/or a nucleic acid encoding the anti-CD33 antibody or antigen binding fragment, wherein the anti-CD33 antibody or antigen binding fragment includes an immunoglobulin heavy chain variable region (V_{H}) comprising SEQ ID NO: 1 and an immunoglobulin light chain variable region (V_{L}) comprising SEQ ID NO: 2. The engineered immune cell of the present technology may further comprise FoxP3 and/or a nucleic acid encoding FoxP3.

The neuronal antigen-specific receptor may be a T cell receptor, a native cell receptor, a non-native cell receptor, or a chimeric antigen receptor (CAR). In some embodiments, the anti-CD33 antibody or antigen binding fragment is secreted. Additionally or alternatively, in some embodiments, the nucleic acid encoding the anti-CD33 antibody or antigen binding fragment comprises a leader sequence for secretion of the anti-CD33 antibody or antigen binding fragment.

Additionally or alternatively, in some embodiments, the chimeric antigen receptor comprises (i) an extracellular antigen binding domain; (ii) a transmembrane domain; and (iii) an intracellular domain. In some embodiments, the extracellular antigen binding domain binds to the neuronal antigen and/or comprises a single chain variable fragment (scFv), such as a human scFv. Additionally or alternatively, in certain embodiments, the extracellular antigen binding domain comprises a signal peptide that is covalently joined to the N-terminus of the extracellular antigen binding domain. In certain embodiments, the transmembrane domain comprises a CD8 transmembrane domain. The intracellular domain may comprise one or more costimulatory domains. Examples of the one or more costimulatory domains include, but are not limited to a CD28 costimulatory domain, a CD3ζ chain, a 4-1BBL costimulatory domain, and any combination thereof.

Additionally or alternatively, in certain embodiments, the anti-CD33 antibody or antigen binding fragment is a scFv, optionally wherein the scFv comprises the amino acid sequence of SEQ ID NO: 3. In some embodiments, the nucleic acid encoding the anti-CD33 antibody or antigen binding fragment comprises SEQ ID NO: 4. The nucleic acid encoding the anti-CD33 antibody or antigen binding fragment may be operably linked to a promoter, such as a constitutive promoter, or a conditional promoter. In certain embodiments, the conditional promoter is inducible by binding of the neuronal antigen-specific receptor. Examples of neuronal antigens include, but are not limited to GD2, GD3, GM1, NCAM, integrin 3, Thy-1, CD44, EGFRvIII, or PDGFR.

In any of the preceding embodiments, the engineered immune cell is a lymphocyte, such as a T cell, a B cell, or a natural killer (NK) cell. In some embodiments, the T cell is a CD4+ T cell or a CD8+ T cell.

In one aspect, the present disclosure provides a mixture of polypeptides comprising a first polypeptide comprising FoxP3 and a chimeric antigen receptor that specifically binds to a neuronal antigen, and a second polypeptide comprising an anti-CD33 antibody, or an antigen binding fragment thereof, wherein the anti-CD33 antibody or antigen binding fragment includes an immunoglobulin heavy chain variable region (V_{H}) comprising SEQ ID NO: 1 and an immunoglobulin light chain variable region (V_{L}) comprising SEQ ID NO: 2. The mixture of polypeptides may further comprise a self-cleaving peptide located between FoxP3 and the chimeric antigen receptor. Examples of self-cleaving peptides include a P2A or a T2A self-cleaving peptide.

Additionally or alternatively, in some embodiments, the second polypeptide comprises a leader sequence for secretion of the anti-CD33 antibody or antigen binding fragment. In some embodiments, the second polypeptide comprises a scFv, optionally wherein the scFv comprises the amino acid sequence of SEQ ID NO: 3. Additionally or alternatively, in certain embodiments, the chimeric antigen receptor comprises (i) an extracellular antigen binding domain; (ii) a transmembrane domain; and (iii) an intracellular domain. In certain embodiments, the extracellular antigen binding domain binds to a neuronal antigen-specific receptor. Examples of neuronal antigens include, but are not limited to GD2, GD3, GM1, NCAM, integrin 3, Thy-1, CD44, EGFRvIII, or PDGFR.

Additionally or alternatively, in some embodiments, the extracellular antigen binding domain comprises a scFv. In certain embodiments, the transmembrane domain comprises a CD8 transmembrane domain and/or the intracellular domain comprises one or more costimulatory domains. Examples of the one or more costimulatory domains include, but are not limited to a CD28 costimulatory domain, a CD3ζ chain, a 4-1BBL costimulatory domain, and any combination thereof.

In one aspect, the present disclosure provides a nucleic acid encoding any and all embodiments of the mixture of polypeptides described herein. The nucleic acid encoding the mixture of polypeptides may be operably linked to a promoter, such as a constitutive promoter, or a conditional promoter. In some embodiments, the conditional promoter is inducible by binding of the neuronal antigen-specific receptor.

In another aspect, the present disclosure provides a vector comprising any and all embodiments of the nucleic acid disclosed herein. The vector may be a viral vector, a retroviral vector, or a plasmid. Also disclosed herein are host cells comprising any and all embodiments of the nucleic acids disclosed herein or any and all embodiments of the vectors disclosed herein.

In certain embodiments, immune cells can be transduced with a vector comprising any and all embodiments of the nucleic acids disclosed herein.

Many expression vectors are available and known to those of skill in the art. The choice of expression vector will be influenced by the choice of host expression system. Such selection is well within the level of skill of the skilled artisan. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector in the cells. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art.

Vectors also can contain additional nucleotide sequences operably linked to the ligated nucleic acid molecule, such as, for example, an epitope tag such as for localization, e.g. a hexa-his tag (SEQ ID NO: 14) or a myc tag, hemagglutinin tag or a tag for purification, for example, a GST fusion, and a sequence for directing protein secretion and/or membrane association.

Expression of the neuronal antigen-specific receptor and/or anti-CD33 antibody or an antigen binding fragment can be controlled by any promoter/enhancer known in the art. Suitable bacterial promoters are well known in the art and described herein below. Other suitable promoters for mammalian cells, yeast cells and insect cells are well known in the art and some are exemplified below. Selection of the promoter used to direct expression of a heterologous nucleic acid depends on the particular application and is within the level of skill of the skilled artisan. Promoters which can be used include but are not limited to eukaryotic expression vectors containing the SV40 early promoter (Bernoist and Chambon, Nature 290:304-310(1981)), the promoter contained in the 3′ long terminal repeat of Rous sarcoma virus (Yamamoto et al. (1980) Cell 22:787-797), the herpes thymidine kinase promoter (Wagner et al. (1981) Proc. Natl. Acad. Sci. USA 75: 1441-1445), the regulatory sequences of the metallothionein gene (Brinster et al. (1982) Nature 296:39-42); prokaryotic expression vectors such as the β-lactamase promoter (Jay et al. (1981) Proc. Natl. Acad. Sci. USA 75:5543) or the tac promoter (DeBoer et al. (1983) Proc. Natl. Acad. Sci. USA 50:21-25); see also “Useful Proteins from Recombinant Bacteria” (1980) in Scientific American 242:79-94); plant expression vectors containing the nopaline synthetase promoter (Herrera-Estrella et al. (1984) Nature 505:209-213) or the cauliflower mosaic virus 35S RNA promoter (Gardner et al. (1981) Nucleic Acids Res. 9:2871), and the promoter of the photosynthetic enzyme ribulose bisphosphate carboxylase (Herrera-Estrella et al. (1984) Nature 510: 115-120); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al. (1984) Cell 55:639-646; Ornitz et al. (1986) Cold Spring Harbor Symp. Quant. Biol. 50:399-409; MacDonald (1987) Hepatology 7:425-515); insulin gene control region which is active in pancreatic beta cells (Hanahan et al. (1985) Nature 515: 115-122), immunoglobulin gene control region which is active in lymphoid cells (Grosschedl et al. (1984) Cell 55:647-658; Adams et al. (1985) Nature 515:533-538; Alexander et al. (1987) Mol. Cell Biol. 7: 1436-1444), mouse mammary tumor virus control region which is active in testicular, breast, lymphoid and mast cells (Leder et al. (1986) Cell 15:485-495), albumin gene control region which is active in liver (Pinckert et al. (1987) Genes and Devel. 1:268-276), alpha-fetoprotein gene control region which is active in liver (Krumlauf et al. (1985) Mol. Cell. Biol. 5:1639-403); Hammer et al. (1987) Science 255:53-58), alpha-1 antitrypsin gene control region which is active in liver (Kelsey et al. (1987) Genes and Devel. 7:161-171), beta globin gene control region which is active in myeloid cells (Magram et al. (1985) Nature 515:338-340); Kollias et al. (1986) Cell 5:89-94), myelin basic protein gene control region which is active in oligodendrocyte cells of the brain (Readhead et al. (1987) Cell 15:703-712), myosin light chain-2 gene control region which is active in skeletal muscle (Shani (1985) Nature 514:283-286), and gonadotrophic releasing hormone gene control region which is active in gonadotrophs of the hypothalamus (Mason et al. (1986) Science 254: 1372-1378).

In addition to the promoter, the expression vector typically contains a transcription unit or expression cassette that contains all the additional elements required for the expression of the heterologous nucleic acid, in host cells. A typical expression cassette contains a promoter operably linked to the gene sequence and signals required for efficient polyadenylation of the transcript, ribosome binding sites and translation termination. Additional elements of the cassette can include enhancers. In addition, the cassette typically contains a transcription termination region downstream of the structural gene to provide for efficient termination. The termination region can be obtained from the same gene as the promoter sequence or can be obtained from different genes.

Some expression systems have markers that provide gene amplification such as thymidine kinase and dihydrofolate reductase. Alternatively, high yield expression systems not involving gene amplification are also suitable, such as using a baculovirus vector in insect cells, with a nucleic acid sequence encoding a polypeptide under the direction of the polyhedron promoter or other strong baculovirus promoter.

Any methods known to those of skill in the art for the insertion of DNA fragments into a vector can be used to construct expression vectors containing a nucleic acid encoding any of the genes disclosed herein. These methods can include in vitro recombinant DNA and synthetic techniques and in vivo recombinants (genetic recombination). The insertion into a cloning vector can, for example, be accomplished by ligating the DNA fragment into a cloning vector which has complementary cohesive termini. If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized nucleic acids encoding restriction endonuclease recognition sequences.

Exemplary plasmid vectors useful to produce the transcripts or polypeptides provided herein contain a strong promoter, such as the HCMV immediate early enhancer/promoter or the MHC class I promoter, an intron to enhance processing of the transcript, such as the HCMV immediate early gene intron A, and a polyadenylation (poly A) signal, such as the late SV40 polyA signal.

Genetic modification of engineered immune cells (e.g., T cells, NK cells) can be accomplished by transducing a substantially homogeneous cell composition with a recombinant DNA or RNA construct. The vector can be a retroviral vector (e.g., gamma retroviral), which is employed for the introduction of the DNA or RNA construct into the host cell genome. For example, a polynucleotide encoding neuronal antigen-specific receptor and/or anti-CD33 antibody or an antigen binding fragment can be cloned into a retroviral vector and expression can be driven from its endogenous promoter, from the retroviral long terminal repeat, or from an alternative internal promoter.

Non-viral vectors or RNA can be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used.

For initial genetic modification of the cells to provide neuronal antigen-specific receptor and/or anti-CD33 antibody or an antigen binding fragment overexpressing immune cells, a retroviral vector can be employed for transduction. However, any other suitable viral vector or non-viral delivery system can be used for genetic modification of cells. For subsequent genetic modification of the cells to provide cells comprising an antigen presenting complex comprising at least two co-stimulatory ligands, retroviral gene transfer (transduction) likewise proves effective. Combinations of retroviral vector and an appropriate packaging line are also suitable, where the capsid proteins will be functional for infecting human cells. Various amphotropic virus-producing cell lines are known, including, but not limited to, PA12 (Miller et al. (1985) Mol. Cell. Biol. 5:431-437); PA317 (Miller et al. (1986) Mol. Cell. Biol. 6:2895-2902); and CRIP (Danos et al. (1988) Proc. Natl. Acad. Sci. USA 85:6460-6464). Non-amphotropic particles are suitable too, e.g., particles pseudotyped with VSVG, RD 114 or GALV envelope and any other known in the art.

Possible methods of transduction also include direct co-culture of the cells with producer cells, e.g., by the method of Bregni et al. (1992) Blood 80: 1418-1422, or culturing with viral supernatant alone or concentrated vector stocks with or without appropriate growth factors and polycations, e.g., by the method of Xu et al. (1994) Exp. Hemat. 22:223-230; and Hughes et al. (1992) J. Clin. Invest. 89: 1817.

Transducing viral vectors can be used to express a co-stimulatory ligand and/or secretes a cytokine (e.g., 4-1BBL and/or IL-12) in an engineered immune cell. In some embodiments, the chosen vector exhibits high efficiency of infection and stable integration and expression (see, e.g., Cayouette et al. (1997) Human Gene Therapy 8:423-430; Kido et al. (1996) Current Eye Research 15:833-844; Bloomer et al. (1997) Journal of Virology 71: 6641-6649; Naldini et al. (1996) Science 272:263 267; and Miyoshi et al. (1997) Proc. Natl. Acad. Sci. U.S.A. 94: 10319,). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller (1990) Human Gene Therapy 15-14; Friedman (1989) Science 244: 1275-1281; Eglitis et al. (1988) BioTechniques 6:608-614; Tolstoshev et al. (1990) Current Opinion in Biotechnology 1:55-61; Sharp (1991) The Lancet 337: 1277-1278; Cornetta et al. (1987) Nucleic Acid Research and Molecular Biology 36:311-322; Anderson (1984) Science 226:401-409; Moen (1991) Blood Cells 17:407-416; Miller et al. (1989) Biotechnology 7:980-990; Le Gal La Salle et al. (1993) Science 259:988-990; and Johnson (1995) Chest 107:77S-83S). Retroviral vectors are particularly well developed and have been used in clinical settings (Rosenberg et al. (1990) N. Engl. J. Med 323:370; Anderson et al., U.S. Pat. No. 5,399,346).

In certain non-limiting embodiments, the vector expressing neuronal antigen-specific receptor and/or anti-CD33 antibody or an antigen binding fragment is a retroviral vector, e.g., an oncoretroviral vector. In some instances, the retroviral vector is a SFG retroviral vector or murine stem cell virus (MSCV) retroviral vector. In certain non-limiting embodiments, the vector expressing a nucleic acid sequence encoding neuronal antigen-specific receptor and/or anti-CD33 antibody or an antigen binding fragment is a lentiviral vector or a transposon vector.

Non-viral approaches can also be employed for the expression of a protein in cell. For example, a nucleic acid molecule can be introduced into a cell by administering the nucleic acid in the presence of lipofection (Feigner et al. (1987) Proc. Nat'l. Acad. Sci. U.S.A. 84:7413; Ono et al. (1990) Neuroscience Letters 17:259; Brigham et al. (1989) Am. J. Med. Sci. 298:278; Staubinger et al. (1983) Methods in Enzymology 101:512), asialoorosomucoid-polylysine conjugation (Wu et al. (1988) Journal of Biological Chemistry 263: 14621; Wu et al. (1989) Journal of Biological Chemistry 264: 16985), or by micro-injection under surgical conditions (Wolff et al. (1990) Science 247: 1465). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g., Zinc finger nucleases, meganucleases, or TALE nucleases). Transient expression can be obtained by RNA electroporation.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g., the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

The engineered immune cells of the presently disclosed subject matter can be cells of the lymphoid lineage or myeloid lineage. Examples of myeloid cells include but are not limited to, mast cells, monocytes, macrophages, dendritic cells, eosinophils, neutrophils, basophils. The lymphoid lineage, comprising B, T, and natural killer (NK) cells, provides for the production of antibodies, regulation of the cellular immune system, detection of foreign agents in the blood, detection of cells foreign to the host, and the like. Non-limiting examples of immune cells of the lymphoid lineage include T cells, Natural Killer (NK) cells, embryonic stem cells, and pluripotent stem cells (e.g., those from which lymphoid cells can be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, T helper cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), Natural killer T cells, Mucosal associated invariant T cells, and T6 T cells. Cytotoxic T cells (CTL or killer T cells) are a subset of T lymphocytes capable of inducing the death of infected somatic or tumor cells.

Natural killer (NK) cells can be lymphocytes that are part of cell-mediated immunity and act during the innate immune response. NK cells do not require prior activation in order to perform their cytotoxic effect on target cells.

The engineered immune cells can be generated from peripheral donor lymphocytes, e.g., those disclosed in Sadelain, M., et al., Nat Rev Cancer 3:35-45 (2003), in Morgan, R. A. et al. (2006) Science 314: 126-129, in Panelli et al. (2000) J Immunol 164:495-504; Panelli et al. (2000) J Immunol 164:4382-4392 (2000), and in Dupont et al. (2005) Cancer Res 65:5417-5427; Papanicolaou et al. (2003) Blood 102:2498-2505. The engineered immune cells (e.g., T cells) can be autologous, non-autologous (e.g., allogeneic), or derived in vitro from engineered progenitor or stem cells.

In certain embodiments, the presently disclosed engineered immune cells (e.g., T cells) expresses from about 1 to about 5, from about 1 to about 4, from about 2 to about 5, from about 2 to about 4, from about 3 to about 5, from about 3 to about 4, from about 4 to about 5, from about 1 to about 2, from about 2 to about 3, from about 3 to about 4, or from about 4 to about 5 vector copy numbers per cell of a heterologous nucleic acid encoding neuronal antigen-specific receptor and/or anti-CD33 antibody or an antigen binding fragment.

The unpurified source of immune cells can be any known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-immune cell initially. Monoclonal antibodies are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. In some embodiments, at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g., plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). In some embodiments, the cells are collected in a medium comprising 2% fetal calf serum (FCS) or 0.2% bovine serum albumin (BSA) or any other suitable, preferably sterile, isotonic medium.

In some embodiments, the engineered immune cells comprise one or more additional modifications. For example, in some embodiments, the engineered immune cells comprise and express (is transduced to express) a chimeric co-stimulatory receptor (CCR). CCR is described in Krause et al. (1998) J. Exp. Med. 188(4):619-626, and US20020018783, the contents of which are incorporated by reference in their entireties. CCRs mimic co-stimulatory signals, but unlike, engineered receptors, do not provide a T-cell activation signal, e.g., CCRs lack a CD3ζ polypeptide. CCRs provide co-stimulation, e.g., a CD28-like signal, in the absence of the natural co-stimulatory ligand on the antigen-presenting cell. A combinatorial antigen recognition, i.e., use of a CCR in combination with an engineered receptor, can augment T-cell reactivity against the dual-antigen expressing T cells, thereby improving selective tumor targeting.

In some embodiments, the engineered immune cells are further modified to suppress expression of one or more genes. In some embodiments, the engineered immune cells are further modified via genome editing. Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, for example, U.S. Pat. Nos. 7,888,121; 7,972,854; 7,914,796; 7,951,925; 8,110,379; 8,409,861; 8,586,526; U.S. Patent Publications 20030232410; 20050208489; 20050026157; 20050064474; 20060063231; 201000218264; 20120017290; 20110265198; 20130137104; 20130122591; 20130177983 and 20130177960, the disclosures of which are incorporated by reference in their entireties. These methods often involve the use of engineered cleavage systems to induce a double strand break (DSB) or a nick in a target DNA sequence such that repair of the break by an error born process such as non-homologous end joining (NHEJ) or repair using a repair template (homology directed repair or HDR) can result in the knock out of a gene or the insertion of a sequence of interest (targeted integration). Cleavage can occur through the use of specific nucleases such as engineered zinc finger nucleases (ZFN), transcription-activator like effector nucleases (TALENs), or using the CRISPR/Cas system with an engineered crRNA/tracr RNA (‘single guide RNA’) to guide specific cleavage. In some embodiments, the engineered immune cells are modified to disrupt or reduce expression of an endogenous T-cell receptor gene (see, e.g. WO 2014153470, which is incorporated by reference in its entirety). In some embodiments, the engineered immune cells are modified to result in disruption or inhibition of PD1, PDL-1 or CTLA-4 (see, e.g. U.S. Patent Publication 20140120622), or other immunosuppressive factors known in the art (Wu et al. (2015) Oncoimmunology 4(7): e1016700, Mahoney et al. (2015) Nature Reviews Drug Discovery 14, 561-584).

### Prophylactic and Therapeutic Methods of the Present Technology

The following discussion is presented by way of example only, and is not intended to be limiting.

“Immunoglobulin-related compositions” as used herein, refers to antibodies (including monoclonal antibodies, polyclonal antibodies, humanized antibodies, chimeric antibodies, recombinant antibodies, multispecific antibodies, bispecific antibodies, etc.,), antibody fragments thereof, and immune cells (e.g., B cells, T cells) that comprise nucleic acids or expression vectors encoding the antibodies and antigen binding fragments. An antibody or antigen binding fragment thereof specifically binds to an antigen. In some embodiments, the immune cells comprise a native T cell receptor (TCR), a non-native TCR, or a CAR.

Therapeutic Methods. One aspect of the present technology includes methods of treating a disease or condition characterized by deposition, accumulation and/or persistence of Aβ plaques.

Additionally or alternatively, in some embodiments, the present technology includes methods of treating Alzheimer's Disease. In some embodiments, the subject is diagnosed as having, suspected as having, or at risk of having a disease or condition characterized by deposition, accumulation and/or persistence of Aβ plaques. Additionally or alternatively, in some embodiments, the subject is suspected of having, is at risk for, or is diagnosed as having late onset Alzheimer's disease, early onset Alzheimer's disease, or intermediate onset Alzheimer's disease. In therapeutic applications, pharmaceutical compositions or medicaments comprising an anti-CD33 immunoglobulin-related composition of the present technology, are administered to a subject suspected of, or already suffering from such a disease or condition (such as, e.g., subjects exhibiting elevated Aβ plaques, aberrant levels/activity of CD33 and/or neuronal cell death compared to a normal control subject, and/or a subject diagnosed with a disease or condition characterized by deposition, accumulation and/or persistence of Aβ plaques, and/or a subject diagnosed with Alzheimer's Disease), in an amount sufficient to cure, or at least partially arrest, the symptoms of the disease, including its complications and intermediate pathological phenotypes in development of the disease.

Subjects suffering from a disease or condition characterized by deposition, accumulation and/or persistence of Aβ plaques and/or subjects suffering from Alzheimer's Disease can be identified by any or a combination of diagnostic or prognostic assays known in the art. For example, typical symptoms of Alzheimer's Disease include, but are not limited to, cognitive dysfunction or decline; memory loss; agitation; mood swings; impaired judgment; dementia; difficulty with abstract thinking; difficulty with familiar tasks; disorientation; diminished communication skills; repetitive speech or actions; impaired visuospatial abilities; impaired speaking, reading, and writing; withdrawal; depression; loss of recognition; loss of motor skills and sense of touch; delusions; paranoia; verbal or physical aggression; and sleep disorders.

In some embodiments, the subject may exhibit elevated Aβ plaques, aberrant levels/activity of CD33 and/or neuronal cell death compared to a normal control subject, which is measureable using techniques known in the art. In some embodiments, the subject may exhibit one or more mutations in APP, PS1, PS2, APOE4, CD33, CLU, BIN1, PICALM, CR1, CD2AP, EPHA1, ABCA7, MS4A4A/MS4A6E and TREM2, which are detectable using techniques known in the art.

In certain embodiments, subjects with a disease or condition characterized by deposition, accumulation and/or persistence of Aβ plaques, and/or subjects suffering from Alzheimer's Disease that are treated with the anti-CD33 immunoglobulin-related compositions of the present technology will show amelioration or elimination of one or more of the following symptoms: cognitive dysfunction or decline; memory loss; agitation; mood swings; impaired judgment; dementia; difficulty with abstract thinking; difficulty with familiar tasks; disorientation; diminished communication skills; repetitive speech or actions; impaired visuospatial abilities; impaired speaking, reading, and writing; withdrawal; depression; loss of recognition; loss of motor skills and sense of touch; delusions; paranoia; verbal or physical aggression; and sleep disorders. In certain embodiments, subjects with a disease or condition characterized by deposition, accumulation and/or persistence of Aβ plaques, and/or Alzheimer's Disease that are treated with the anti-CD33 immunoglobulin-related compositions of the present technology will show reduced CD33 expression/activity (e.g., at the cell surface of microglia), decreased neuronal cell death, and/or increased amyloid beta (Aβ) uptake by microglia cells compared to untreated Alzheimer's Disease subjects.

Prophylactic Methods. In one aspect, the present technology provides a method for preventing or delaying the onset of a disease or condition characterized by deposition, accumulation and/or persistence of Aβ plaques. Additionally or alternatively, in some aspects, the present technology provides a method for preventing or delaying the onset Alzheimer's Disease.

Subjects at risk for aberrant levels/activity of CD33 and/or neuronal cell death compared to a normal control subject, include those at risk or susceptible to a disease or condition characterized by deposition, accumulation and/or persistence of Aβ plaques, and/or a subject at risk or susceptible to Alzheimer's Disease. Such subjects can be identified by, e.g., any or a combination of diagnostic or prognostic assays known in the art.

In prophylactic applications, pharmaceutical compositions or medicaments of anti-CD33 immunoglobulin-related compositions of the present technology, are administered to a subject susceptible to, or otherwise at risk of a disease or condition characterized by deposition, accumulation and/or persistence of Aβ plaques, and/or a subject susceptible to, or otherwise at risk of Alzheimer's Disease, in an amount sufficient to eliminate or reduce the risk, or delay the onset of the disease, including biochemical, histologic and/or behavioral symptoms of the disease, its complications and intermediate pathological phenotypes presenting during development of the disease.

Administration of prophylactic anti-CD33 immunoglobulin-related compositions of the present technology can occur prior to the manifestation of symptoms characteristic of the disease or disorder, such that the disease or disorder is prevented or, alternatively, delayed in its progression. In some embodiments, subjects at risk for aberrant levels and/or function of CD33 and/or neuronal cell death compared to a normal control subject, are those at risk for, or susceptible to a disease or condition characterized by deposition, accumulation and/or persistence of Aβ plaques, and/or a subjects at risk for or susceptible to Alzheimer's Disease.

In some embodiments, treatment with the anti-CD33 immunoglobulin-related compositions of the present technology will prevent or delay the onset of one or more of the following symptoms: cognitive dysfunction or decline; memory loss; agitation; mood swings; impaired judgment; dementia; difficulty with abstract thinking; difficulty with familiar tasks; disorientation; diminished communication skills; repetitive speech or actions; impaired visuospatial abilities; impaired speaking, reading, and writing; withdrawal; depression; loss of recognition; loss of motor skills and sense of touch; delusions; paranoia; verbal or physical aggression; and sleep disorders. In certain embodiments, treatment with the anti-CD33 immunoglobulin-related compositions of the present technology will show CD33 expression/activity (e.g., at the cell surface of microglia), neuronal cell death, and/or amyloid beta (Aβ) uptake by microglia cells that resembles those observed in healthy controls.

For therapeutic and/or prophylactic applications, a pharmaceutical composition comprising an anti-CD33 immunoglobulin-related composition of the present technology, is administered to the subject. In some embodiments, the anti-CD33 immunoglobulin-related composition of the present technology is administered one, two, three, four, or five times per day. In some embodiments, the anti-CD33 immunoglobulin-related composition of the present technology is administered more than five times per day. Additionally or alternatively, in some embodiments, the anti-CD33 immunoglobulin-related composition of the present technology is administered every day, every other day, every third day, every fourth day, every fifth day, or every sixth day. In some embodiments, the anti-CD33 immunoglobulin-related composition of the present technology is administered weekly, bi-weekly, tri-weekly, or monthly. In some embodiments, the anti-CD33 immunoglobulin-related composition of the present technology is administered for a period of one, two, three, four, or five weeks. In some embodiments, the anti-CD33 immunoglobulin-related composition is administered for six weeks or more. In some embodiments, the anti-CD33 immunoglobulin-related composition is administered for twelve weeks or more. In some embodiments, the anti-CD33 immunoglobulin-related composition is administered for a period of less than one year. In some embodiments, the anti-CD33 immunoglobulin-related composition is administered for a period of more than one year. In some embodiments, the anti-CD33 immunoglobulin-related composition is administered throughout the subject's life.

In some embodiments of the methods of the present technology, the anti-CD33 immunoglobulin-related composition of the present technology is administered daily for 1 week or more. In some embodiments of the methods of the present technology, the anti-CD33 immunoglobulin-related composition of the present technology is administered daily for 2 weeks or more. In some embodiments of the methods of the present technology, the anti-CD33 immunoglobulin-related composition of the present technology is administered daily for 3 weeks or more. In some embodiments of the methods of the present technology, the anti-CD33 immunoglobulin-related composition of the present technology is administered daily for 4 weeks or more. In some embodiments of the methods of the present technology, the anti-CD33 immunoglobulin-related composition of the present technology is administered daily for 6 weeks or more. In some embodiments of the methods of the present technology, the anti-CD33 immunoglobulin-related composition of the present technology is administered daily for 12 weeks or more. In some embodiments, the anti-CD33 immunoglobulin-related composition is administered throughout the subject's life.

In some embodiments, the anti-CD33 immunoglobulin-related compositions of the present technology, may be combined with one or more additional therapies for the prevention or treatment of a disease or condition characterized by deposition, accumulation and/or persistence of Aβ plaques. In some embodiments, the anti-CD33 immunoglobulin-related compositions, may be combined with one or more additional therapies for the prevention or treatment of Alzheimer's Disease. Additional therapeutic agents include, but are not limited to, donepezil, galantamine, memantine, rivastigmine, memantine extended-release and donepezil (Namzaric), aducanumab, solanezumab, insulin, verubecestat, AADvac1, CSP-1103, and intepirdine.

In any case, the multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

### Modes of Administration, Dosages And Formulation

Any method known to those in the art for contacting a cell, organ or tissue with an anti-CD33 immunoglobulin-related composition of the present technology, may be employed. Suitable methods include in vitro, ex vivo, or in vivo methods. In vivo methods typically include the administration of an anti-CD33 immunoglobulin-related composition of the present technology, such as those described above, to a mammal, suitably a human. When used in vivo for therapy, the anti-CD33 immunoglobulin-related compositions of the present technology, are administered to the subject in effective amounts (i.e., amounts that have desired therapeutic effect). The dose and dosage regimen will depend upon the degree of the disease in the subject, the characteristics of the particular anti-CD33 immunoglobulin-related composition of the present technology used, e.g., its therapeutic index, the subject, and the subject's history.

The effective amount may be determined during pre-clinical trials and clinical trials by methods familiar to physicians and clinicians. An effective amount of an anti-CD33 immunoglobulin-related composition useful in the methods may be administered to a mammal in need thereof by any of a number of well-known methods for administering pharmaceutical compounds. The anti-CD33 immunoglobulin-related composition may be administered systemically or locally.

According to the methods of the present technology, the anti-CD33 immunoglobulin-related composition can be incorporated into pharmaceutical compositions suitable for administration. The pharmaceutical compositions generally comprise recombinant or substantially purified immunoglobulin-related composition and a pharmaceutically-acceptable carrier in a form suitable for administration to a subject. Pharmaceutically-acceptable carriers are determined in part by the particular composition being administered, as well as by the particular method used to administer the composition. Accordingly, there is a wide variety of suitable formulations of pharmaceutical compositions for administering the immunoglobulin-related compositions (See, e.g., Remington's Pharmaceutical Sciences, Mack Publishing Co., Easton, PA 18^{th} ed., 1990). The pharmaceutical compositions are generally formulated as sterile, substantially isotonic and in full compliance with all Good Manufacturing Practice (GMP) regulations of the U.S. Food and Drug Administration.

The terms “pharmaceutically-acceptable,” “physiologically-tolerable,” and grammatical variations thereof, as they refer to compositions, carriers, diluents and reagents, are used interchangeably and represent that the materials are capable of administration to or upon a subject without the production of undesirable physiological effects to a degree that would prohibit administration of the composition. For example, “pharmaceutically-acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. “Pharmaceutically-acceptable salts and esters” means salts and esters that are pharmaceutically-acceptable and have the desired pharmacological properties. Such salts include salts that can be formed where acidic protons present in the composition are capable of reacting with inorganic or organic bases. Suitable inorganic salts include those formed with the alkali metals, e.g., sodium and potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically-acceptable esters include esters formed from carboxy, sulfonyloxy, and phosphonoxy groups present in the anti-CD33 immunoglobulin-related composition, e.g., C₁₋₆ alkyl esters. When there are two acidic groups present, a pharmaceutically-acceptable salt or ester can be a mono-acid-mono-salt or ester or a di-salt or ester; and similarly where there are more than two acidic groups present, some or all of such groups can be salified or esterified. An anti-CD33 immunoglobulin-related composition named in this technology can be present in unsalified or unesterified form, or in salified and/or esterified form, and the naming of such anti-CD33 immunoglobulin-related composition is intended to include both the original (unsalified and unesterified) compound and its pharmaceutically-acceptable salts and esters. Also, certain embodiments of the present technology can be present in more than one stereoisomeric form, and the naming of such anti-CD33 immunoglobulin-related composition is intended to include all single stereoisomers and all mixtures (whether racemic or otherwise) of such stereoisomers. A person of ordinary skill in the art, would have no difficulty determining the appropriate timing, sequence and dosages of administration for particular drugs and compositions of the present technology.

The anti-CD33 immunoglobulin-related composition of the present technology described herein, can be incorporated into pharmaceutical compositions for administration, singly or in combination, to a subject for the treatment or prevention of a disease described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Examples of such carriers or diluents include, but are not limited to, water, saline, Ringer's solutions, dextrose solution, and 5% human serum albumin. Liposomes and non-aqueous vehicles such as fixed oils may also be used. The use of such media and compounds for pharmaceutically active substances is well known in the art. Except insofar as any conventional media or compound is incompatible with the anti-CD33 immunoglobulin-related composition, use thereof in the compositions is contemplated. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The anti-CD33 immunoglobulin-related compositions of the present technology can include a carrier, which can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof. The immunoglobulin-related compositions of the present technology can be administered in the form of a depot injection or implant preparation which can be formulated in such a manner as to permit a sustained or pulsatile release of the active ingredient.

Alternatively, anti-CD33 immunoglobulin-related compositions can be administered as a sustained release formulation, in which case less frequent administration is required. Dosage and frequency vary depending on the half-life of the composition in the subject. The dosage and frequency of administration can vary depending on whether the treatment is prophylactic or therapeutic. In prophylactic applications, a relatively low dosage is administered at relatively infrequent intervals over a long period of time.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic protein or peptide can be formulated in a carrier system. The carrier can be a colloidal system. The colloidal system can be a liposome, a phospholipid bilayer vehicle. In one embodiment, the therapeutic protein is encapsulated in a liposome while maintaining peptide integrity. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic protein can be embedded in the polymer matrix, while maintaining protein integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi, et al., Cancer Lett., 100:63-69 (1996), describes the use of fusogenic liposomes to deliver a protein to cells both in vivo and in vitro.

Toxicity. Optimally, an effective amount (e.g., dose) of an anti-CD33 immunoglobulin-related composition described herein will provide therapeutic benefit without causing substantial toxicity to the subject. Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. The exact formulation, route of administration and dosage can be chosen by the individual physician in view of the subject's condition. See, e.g., Fingl et al., In: The Pharmacological Basis of Therapeutics, Ch. 1 (1975). For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the anti-CD33 immunoglobulin-related composition of the present technology, sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of immunoglobulin-related composition ranges from 0.001-10,000 micrograms per kg body weight.

In one embodiment, anti-CD33 immunoglobulin-related composition concentrations in a carrier range from 0.2 to 2000 micrograms per delivered milliliter. An exemplary treatment regime entails administration once per day, once a week, once per every two weeks or once a month or once every 3 to 6 months. Anti-CD33 immunoglobulin-related compositions may be administered on multiple occasions. Intervals between single dosages can be hourly, daily, weekly, monthly or yearly. Intervals can also be irregular as indicated by measuring blood levels of the immunoglobulin-related composition in the subject. In some methods, dosage is adjusted to achieve a serum antibody concentration in the subject of from about 75 μg/mL to about 125 μg/mL, 100 g/mL to about 150 μg/mL, from about 125 μg/mL to about 175 μg/mL, or from about 150 g/mL to about 200 μg/mL.

In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of an anti-CD33 immunoglobulin-related composition of the present technology may be defined as a concentration of the composition at the target tissue of 10⁻¹² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

The anti-CD33 immunoglobulin-related compositions of the present technology may optionally be administered as a single bolus to a subject in need thereof. Alternatively, the dosing regimen may comprise multiple administrations performed at various times after the appearance of Aβ plaques. In some embodiments, the anti-CD33 immunoglobulin-related compositions of the present technology comprise pharmaceutical formulations which may be administered to subjects in need thereof in one or more doses. Dosage regimens can be adjusted to provide the desired response (e.g., a therapeutic response).

### Kits

The present technology provides kits for decreasing, inhibiting or reducing brain beta amyloid (Aβ) accumulation or persistence in a subject in need thereof or for preventing or treating Alzheimer's Disease, comprising at least one immunoglobulin-related composition of the present technology or a functional variant (e.g., substitutional variant) thereof. Optionally, the above described components of the kits of the present technology are packed in suitable containers and labeled for treatment of Alzheimer's Disease. The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. The kits may optionally include instructions customarily included in commercial packages of therapeutic or diagnostic products, that contain information about, for example, the indications, usage, dosage, manufacture, administration, contraindications and/or warnings concerning the use of such therapeutic or diagnostic products.

The kit can also comprise, e.g., a buffering agent, a preservative or a protein-stabilizing agent. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit, e.g., for treatment of Alzheimer's disease in a subject in need thereof. In certain embodiments, the use of the reagents can be according to the methods of the present technology.

### EXAMPLES

The present technology is further illustrated by the following Examples, which should not be construed as limiting in any way.

### Example 1: Materials and Methods

Purification of endotoxin free Aβ peptides. ClearColi BL21 (Lucigen, Middleton, WI) bacteria were transformed with pET28 His-Sumo-Aβ and grown in LB broth supplemented with 50 μg/ml Kanamycin. Overexpression was initiated with 250 μM IPTG when OD 600 nm reached 0.6 and bacterial cells were incubated overnight at 15° C. Bacteria were collected and lysed with French press (Thermo Fisher, Waltham, MA) in Tris 25 mM, 300 mM NaCl, 25 M Imidazole, pH=8.0, lysozyme 100 μg/ml, 1 pill of protease inhibitor and 10 μl of DNAse and loaded on Histrap Ni column (Cytiva, Marlborough, MA) on Akta Pure (Cytiva, Marlborough, MA). Elution was performed with 25 mM Tris, 300 mM NaCl, 250 mM Imidazole, pH=8.0 and the eluted sample was incubated with 1-unit ULP (Sigma-Adlrich, St. Louis, MO) for Sumo cleavage and dialyzed against Tris 25 mM, 0 mM NaCl, pH=8.0. Cleaved sample containing separated His-Sumo and Aβ was loaded on an anion exchange Hitrap MonoQ column (Cytiva, Marlborough, MA) on Akta Pure (Cytiva, Marlborough, MA) and eluted with Tris 25 mM, 1 M NaCl, pH=8.0 gradient. Fractions were collected and analyzed with 16.5% Tris-Tricine SDS-PAGE and the fractions corresponding to Aβ molecular weight were collected.

HEK-Blue endotoxin test. Detection of secreted embryonic alkaline phosphatase (SEAP) was done using p-nitrophenol phosphate (pNPP) substrate (Sigma-Adlrich, St. Louis, MO). Briefly, 10,000 HEK Blue (Invitrogen, Carlsbad, CA) in DMEM supplemented with 10% FBS+ Pen/Strep were seeded in 96 well plates overnight. Cells were then incubated with various concentrations of LPS (Sigma-Adlrich, St. Louis, MO), purified A040 or Aβ42 overnight and cell media were collected. pNPP substrate was added to media in a 1:1 ratio, and SEAP activity was determined by reading the OD at 405 nm in plate reader.

TNFα ELISA. 10,000 PMA (100 ng/ml) treated THP-1 monocyte cells in RPMI supplemented with 10% FBS+ Pen/Strep were seeded in a 96 well plate overnight. After incubation with LPS, purified A040 and Aβ42 in different concentrations for 24 hours, media were collected. TNFα detection was performed using ELISA MAX™ Deluxe Set Human TNFα (Biolegend, San Diego, CA) according to manufacturer's instruction.

α-CD33 (Hu195) treatment, Aβ42 uptake and Aβ42 ELISA. PMA treated THP-1 cells, 10,000 (96 well plate), 60,000 (24 wells), 100,000 (12 wells) and 250,000 (6 wells) were seeded overnight. Attached cells were pre-incubated with PBS, IgG control or α-CD33 (Hu195) for the indicated hours or 4 hours prior to addition of 1 M Aβ42. After 3 hours incubation, cells were washed and replaced with fresh media for 3 hours to allow internalization, then lysed with RIPA+ protease inhibitor and tested with Amyloid beta 42 Human ELISA Kit (Thermo Fisher, Waltham, MA) according to manufacturer's instructions.

Human+CD14 monocyte purification. Healthy donor blood was drawn and after ficoll isolation of peripheral blood mononuclear cell (PBMC), cells were incubated with α-CD14 magnetic beads (Miltenyi Biotec, Bergisch Gladbach, Germany) and passed through magnetic separation to dispose of-CD14 cells. The magnetic bound cells eluted were the +CD14 monocytes, which were seeded 200,000 per well in a 6 well plate. Seeded cells were pre-incubated for 4 hours with Hu195, IgG control and no treatment followed by 3 hours 1 M Aβ42 incubation. After incubation cells were washed and replaced with fresh media for additional 3 hours to allow internalization, then lysed with RIPA+ protease inhibitor and tested with Amyloid beta 42 Human ELISA Kit.

pH sensitive Rhodamine (pHrodo)-Aβ42 conjugation. Succinimidyl ester pHrodo Red dye (Thermo Fisher, Waltham, MA) was conjugated to purified Aβ42 or synthetic Aβ42 (AnaSpec, Fremont, CA) at pH=8.5 in molar ratio 1:20 (peptide to dye) for 1 hour at 25° C. Unconjugated dye was separated with dialysis using 3 kDa membrane against PBS followed by spin column with 3 kDa cutoff to remove excessive pHrodo dye.

Live imaging of pHrodo-Aβ42 uptake. THP-1 cell (10,000), ES microglia (20,000) or co-cultured ES Microglia (20,000) and neurons (60,000) were seeded in 96 wells and pre-incubated with different treatments and negative control 0.5 μM Latrunculin (phagocytic inhibitor) (Cyman Chem, Ann Arbor, MI) for 4 hours prior to addition of 1 μM pHrodo-Aβ42. Images were continuously taken every 1 or 2 hours with brightfield and RFP channel (10×objective) using Cytation 5 (Biotek, Winooski, VT). Quantification of continuous live imaging was performed with the Gen5 software, RFP intensity (A.U.) was measured after masking for individual cells.

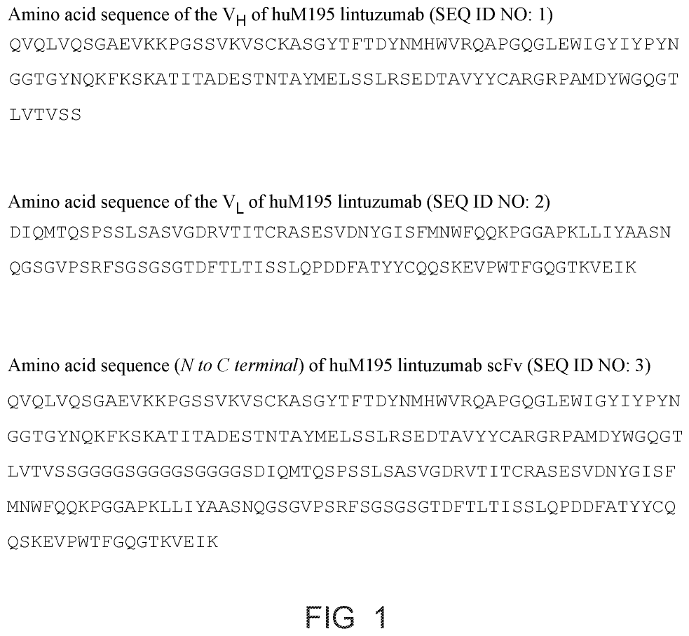

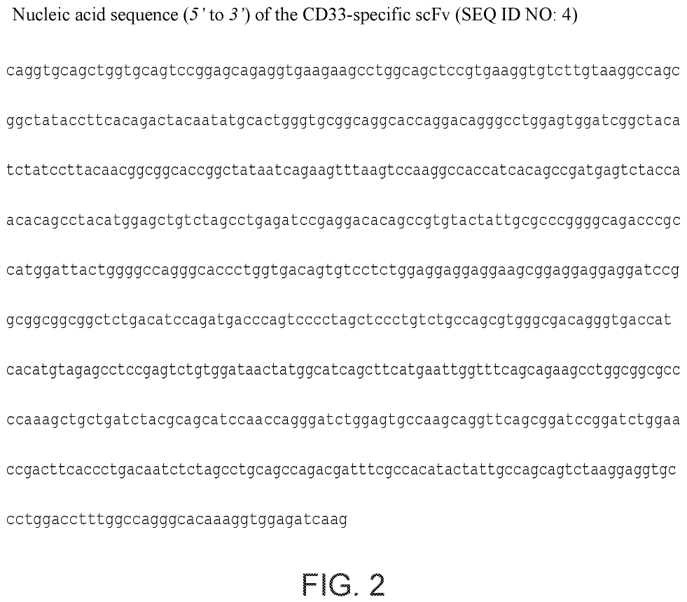

Purification of Hu195 single chain variable fragment (scFv). The Hu195 scFv amino acid and nucleic acid sequences are disclosed in FIGS. 1-2. The gene was cloned into the pSF-CMV-Puro-IL2 secretion plasmid (Sigma-Adlrich, St. Louis, MO) with a C-terminal 8×His-tag (SEQ ID NO: 15). FreeStyle 293 cells were grown in serum-free Expression Medium (Thermo Fisher, USA) at 37° C., 8% CO₂ on a platform shaker set to 225 RPM. Cells were seeded at a density of 1.6×10⁶ cells/mL. Cells were then transiently transfected with 100 g pSF-IL2-CD33scFV-H6 plasmid and incubated overnight. Next day, 15 mL of nutrient-rich serum-free medium (Expression Medium with 20% tryptone and 0.5M valproate) was added and cells were grown untouched until day 4 after transfection. The medium was collected, filtered and loaded on a Histrap column (Cytiva, Marlborough, MA) on the Akta Pure (Cytiva, Marlborough, MA).

Equilibrium dissociation constant (Kd) measurement with bio-layer interferometry (BLI) biosensor. Human CD33-FC protein (R&D Systems, Minneapolis, MN) was captured on Anti-hIgG Fc Capture (AHC) Biosensors (ForteBio, Fremont, CA) overnight and blocked with FC. Different concentrations of Hu195 antibody or Hu195 scFv were run on the Octet RED96e instrument, which measured the reflected white light. The binding graphs were fitted to a Kd constant model.

Western blot and antibodies. Cells were treated with PBS control, control antibody (1 μg/ml), Hu195 antibody (1 μg/ml), control scFv (1 μg/ml) or Hu195 scFv (1 g/ml) for 24 hours and lysed in RIPA+ protease inhibitors. Samples were separated in 4-20% SDS-PAGE, transferred to polyvinylidene difluoride (PVDF) membrane and blotted with rabbit α-CD33 (Abcam, Cambridge, United Kingdom), human IgG HRP (Abcam, Cambridge, United Kingdom), rabbit phosphor-SHIP1(Abcam, Cambridge, United Kingdom) or mouse αH3 (Abcam, Cambridge, United Kingdom) antibodies overnight. Corresponding secondary HRP conjugated antibodies were added and detection was carried out with enhanced chemiluminescence (ECL).

Flow cytometry. THP-1 cells treated for the indicated time points with PBS (no treatment), Hu195 antibody (1 μg/ml), Hu195 scFv (1 μg/ml) and labeled with commercially available FITC conjugated α-CD33 antibody (Biolegend, San Diego, CA). Samples were run and analyzed in Fortessa LSRII flow cytometry instrument (BD Biosciences, San Jose, CA).

Human Phospho-Immunoreceptor Array Kit. THP-1 cells (1,000,000 cells in T75) were treated with PBS or Hu195 antibody (1 μg/ml) or with PBS or scFv Hu195 (1 g/ml) for 1 min or 5 hours. Immunoreceptor phosphorylation was detected using Human Phospho-Immunoreceptor Array kit (R&D Systems, Minneapolis, MN) according to manufacturer's instructions. The dot blot intensity corresponding to phosphorylated-CD33 was analyzed and quantified using ImageJ.

Proximity ligation assay. Mouse α-CD33 primary antibodies (Biolegend, San Diego, CA) were conjugated with − or + DNA probes using Duolink PLA Probemaker (Sigma-Adlrich, St. Louis, MO) according to manufacturer's instructions. CD33 dimerization was determined by proximity ligation assay in HL60 cells. After 1 hour of treatment, cells were fixed with 4% PFA and permeabilized in 0.2% Tween-20, and proximity ligation assay (Sigma-Adlrich, St. Louis, MO) was performed according to manufacturer's instructions. After final washes, cells were stained with Alexa Fluor 488 Tubulin (Invitrogen, Carlsbad, CA), mounted with a mounting medium containing DAPI (Invitrogen, Carlsbad, CA) and PLA signals were imaged by Axio Imager Z2 (ZEISS, Oberkochen, Germany)

### Example 2: Lintuzumab Antibody and scFv Treatment Increase A42 Uptake by Reducing CD33

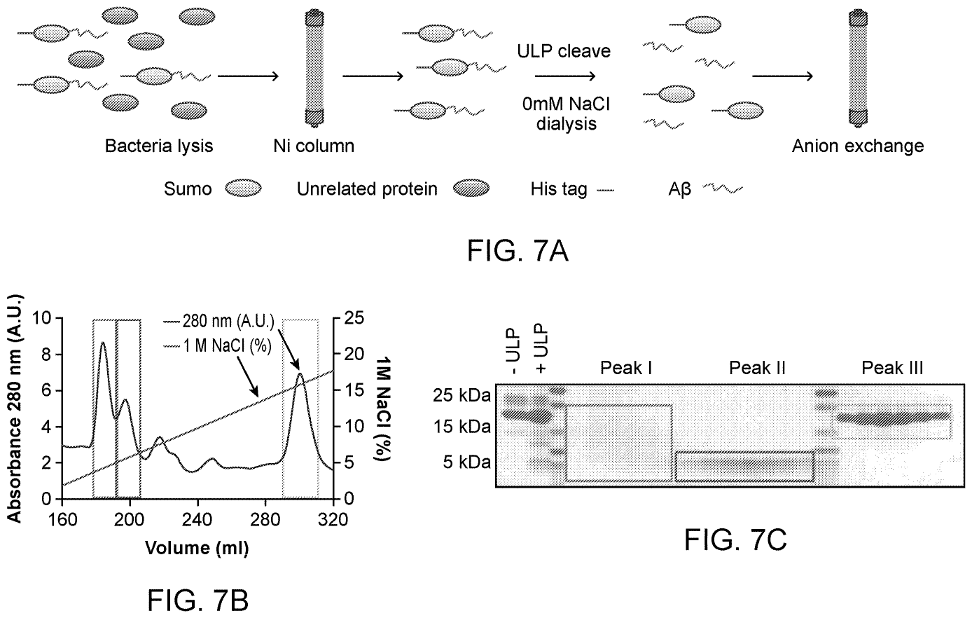

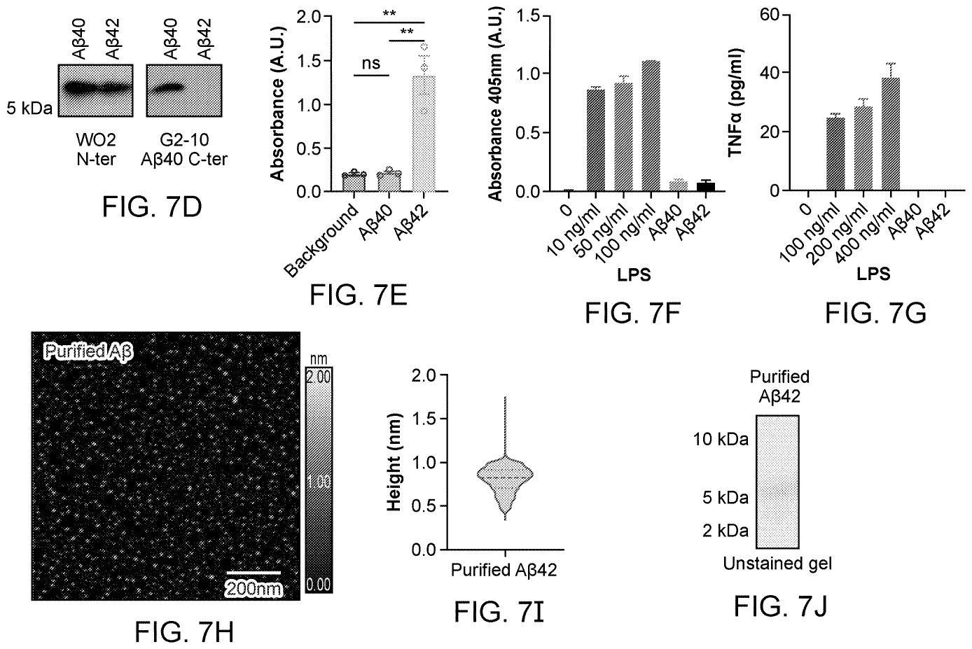

To test the efficacy of Lintuzumab (HuM195) in enhancing Aβ uptake, we first created a stable Aβ peptide and reliable assay which allows a real time monitoring of the uptake process. Commercially synthetic Aβ peptide require intensive treatment to yield a soluble sample and often suffer from inconsistence batch to batch variation (10). We have developed a two-step purification protocol of endotoxin free Aβ peptides using expression in E. Coli cells depleted of LPS (clear coli) (FIGS. 7A-7B). The Aβ product is about <95% pure with no apparent impurity bands as seen in SDS-PAGE analysis (FIG. 7C). The purified Aβ peptides were recognized by Aβ antibody (W02) and A040 was specifically recognized by C-terminal Aβ antibody (G2-10), but not Aβ42 (FIG. 7D). In addition, a commercial Aβ42 ELISA kit was able to detect Aβ42 only (FIG. 7E), confirming the purified Aβ peptides contained the specific sequence. In addition, the purified Aβ peptides had no bacterial product-related activities (FIG. 7F) as determined by the alkaline phosphatase activity from HEK blue cells; and the peptides did not stimulate pro-inflammatory cytokine responses as measured by TNFα secretion from THP-1 monocyte cells compared to LPS (FIG. 7G).

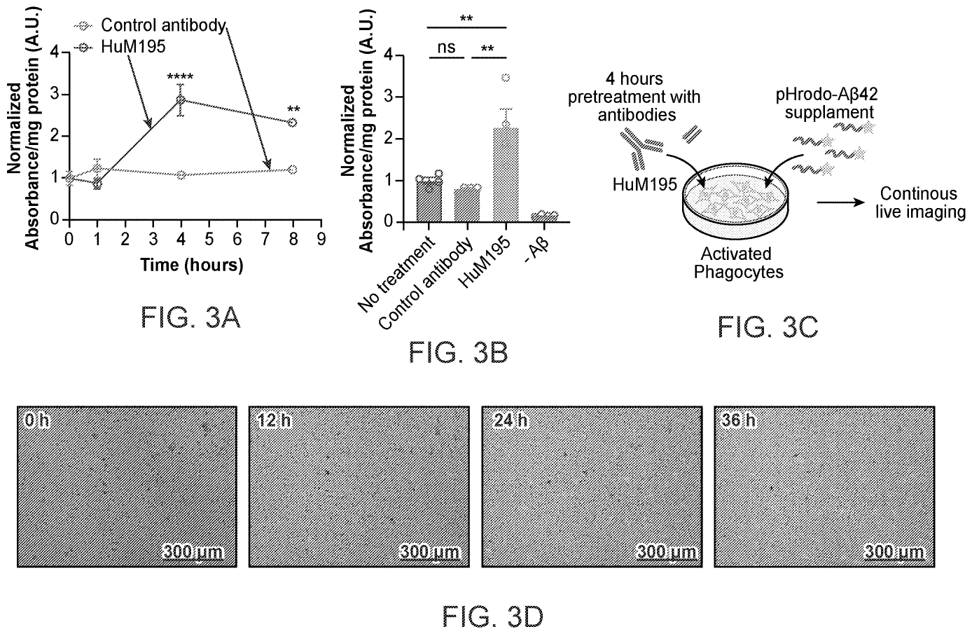

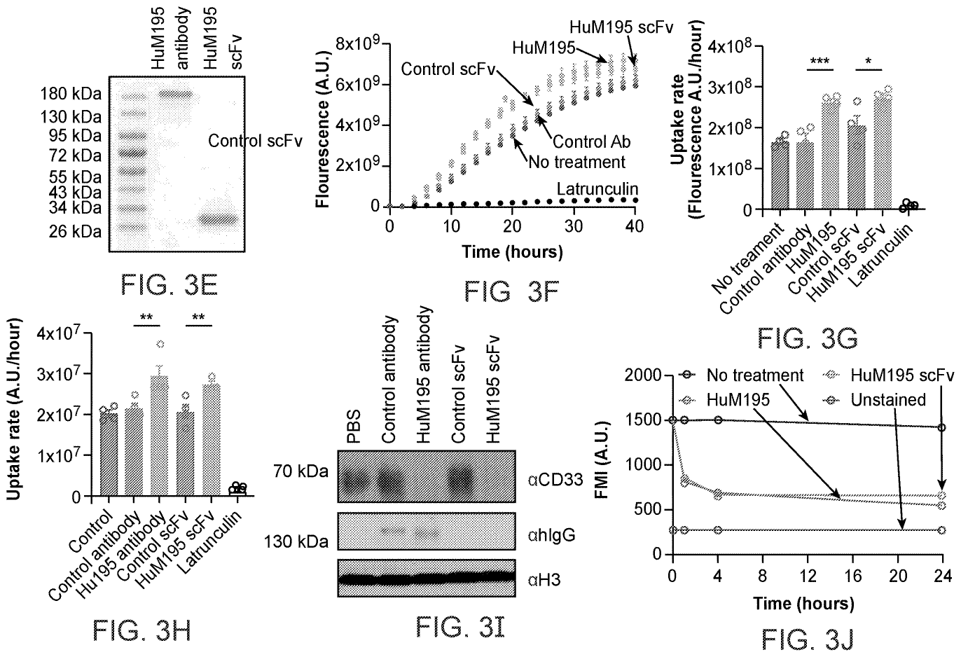

Next, we tested the effect of HuM195 on purified Aβ42 uptake by phagocytic cells. First, the optimal incubation time of HuM195 with cells was determined. We pretreated PMA activated THP1 monocytic cells with HuM195 or control antibody (1 g/ml) for up to 24 hours followed by the addition of purified Aβ42 (1 μM). Cells were given 3 hours to uptake Aβ peptides, followed by 1 hour, then were washed and lysed. Finally, the internalized Aβ42 levels were measured by ELISA kit. Pre-incubation with HuM195 increased Aβ42 uptake by THP-1 with saturated effect after 4 hours compare to IgG control (FIG. 3A). To assess the effect of HuM195 in primary cells lines, we purified CD14+ cells from peripheral blood mononuclear cells (PBMC) from healthy donors. Like the THP-1 experiment, HuM195 treatment significantly increased Aβ42 uptake in human primary CD14+ monocyte compared to IgG controls (FIG. 3B). Thus, we determined the minimum time for HuM195 to activate phagocytic cells to be approximately 4 hours.

It is known that Aβ peptides tend to bind biomolecules non-specifically, including cell membrane (11). Therefore, ELISA assay may provide a less accurate measurement of true “internalization” as the Aβ could bind to the cell surface without getting internalized. To improve the detection of internalized Aβ42 thus tracking the phagocytosis process, we conjugated a pH sensitive Rhodamine Red to Aβ42 (pHrodo-Aβ42). During the uptake process, phagocytic cell actively internalize pHrodo-Aβ42 in vacuoles which will fuse with low pH lysosome for degradation, thereby activating the pHrodo dye to emit its fluorescent signal (12). AFM analysis of the purified Aβ42 showed a homogeneous oligomer size corresponding to the expected dimensions (FIGS. 711-7I). In addition, high efficacy of pHrodo dye conjugation is observed by unstained SDS PAGE (FIG. 7J). We next tested the pHrodo-Aβ42 uptake in dynamic real time (FIG. 3C). pHrodo-Aβ42 (1 μM) was add to embryonic stem (ES) cell derived microglia and images were taken immediately using Cytation 5 in 2-hour interval. As expected, there was a steady increase in fluorescent intensity signal with time reflecting the continues phagocytosis of pHrodo-Aβ42 (FIG. 3D).

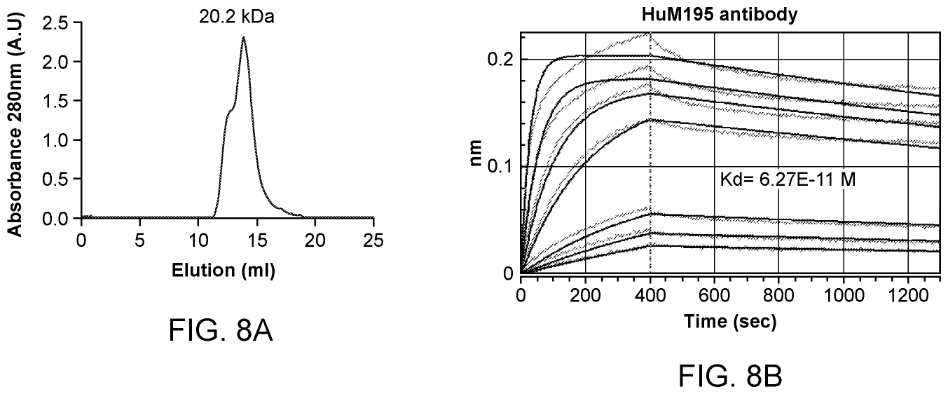

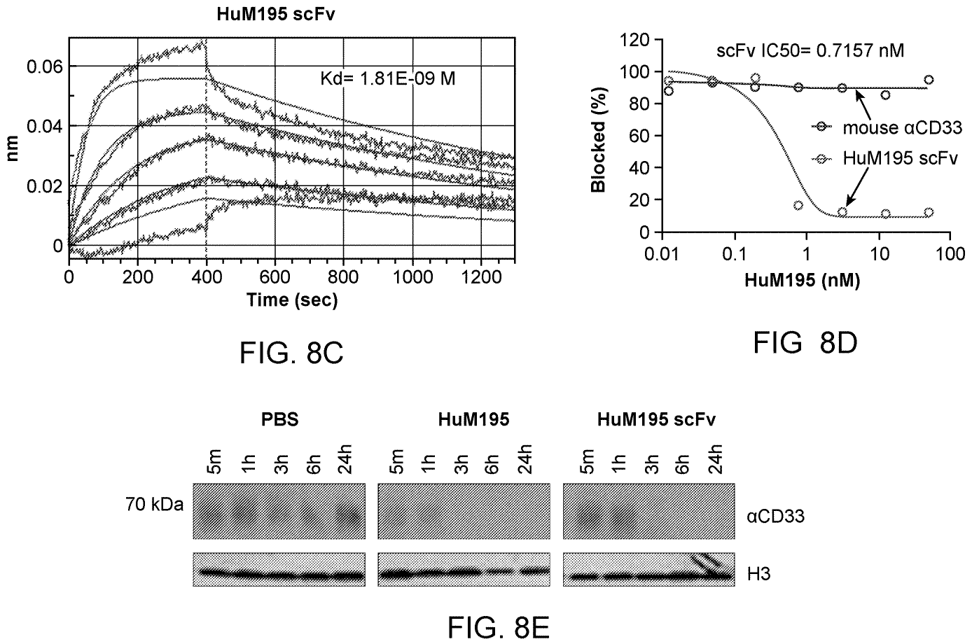

The central nerve system (CNS) is protected from the entry of pathogens, circulating immune cells and blood factor, by a physical blood-brain barrier (BBB) (13). Although the brain was once thought to be immune-privileged tissue, it is now well established that a crosstalk between CNS and the peripheral immune system occurs (14). Nevertheless, macromolecules such as antibodies are still believed to have only limited access to the CNS (15). Therefore, we generated a single-chain variable fragment (scFv) derived from HuM195 antibody which due to its smaller size have better tissue penetration (16). We expressed and purified the HuM195 scFv with a His-tag in a expression mammalian system. The molecular weight of the purified product corresponding to the expected size (˜25 kDa) with >98% purity (FIG. 3E). In addition, size exclusion chromatography determined that the HuM195 scFv is in a monomeric form (FIG. 8A). Since the scFv contains only one binding site compared to the bivalent antibody, we determined its binding properties and ability to recapitulate the functions of the full length HuM195 IgG. Although the equilibrium dissociation constant (Kd) of HuM195 antibody (6.27×10⁻¹¹ M) is ˜30 fold higher than the HuM195 scFv (1.81×10⁻⁰⁹ M) (FIGS. 8B-8C), the nanomolar range Kd value of the scFv still makes it a highly effective binder. As expected, the HuM195 antibody and scFv shared similar CD33 binding site as shown in the competitive ELISA assay (FIG. 8D). Next, we tested the effect of HuM195 scFv on pHrodo-Aβ42 uptake by ES microglia. Both, HuM195 antibody (1 μg/ml) and HuM195 scFv (1 μg/ml) treatment exhibited similar increases in Aβ42 uptake in ES derived microglia compared to the control counterparts (FIGS. 3F-3G). Together, these data demonstrate that the Aβ42 uptake live quantification imaging can detect changes in phagocytic abilities promoted by both forms of HuM195.

Antibodies against cell surface antigens may be internalized through their specific interactions with the target and in some cases may induce ligand internalization (17). HuM195 binding results in CD33 internalization and degradation and this property was used for antibody-drug conjugate in AML treatment (18). To investigate whether the HuM195 scFv acted in similar mechanism as the full-length antibody we measured cell surface and cell associated CD33 after HuM195 antibody or scFv treatment. We first examine the effect of HuM195 antibody and scFv in PMA activated THP1 with our pHrodo-Aβ42 uptake assay. Like the ES microglia experiment above, treatment with HuM195 antibody or scFv increase Aβ42 uptake compared to the control antibody or scFv, respectively (FIG. 3H). Interestingly, western blot analysis of CD33 in THP-1 treated cells revealed that both HuM195 antibody and HuM195 scFv reduces total CD33 in cell to undetectable levels after 4 hours (FIG. 3I). Time course degradation experiments determined that treatment with HuM195 antibody or scFv between 1-3 hours is sufficient to completely reduce CD33 in cells (FIG. 8E). Moreover, both HuM195 full-length antibody and scFv significantly reduce surface CD33 in time dependent manner and reaches maximal effect after 4 hours (FIG. 3J). Surprisingly, these data indicated that 4 hours treatment even with the monovalent scFv results in complete degradation of CD33 which correspond with the maximal effect of HuM195 on Aβ uptake increase (FIG. 3A).

These results demonstrate that the anti-CD33 immunoglobulin-related compositions of the present technology are useful in methods for treating or preventing Alzheimer's disease and/or reducing brain accumulation and/or persistence of Aβ plaques in a subject in need thereof.

### Example 3: HuM195 Antibody Temporarily Induces Phosphorylation Signaling by CD33 Dimerization

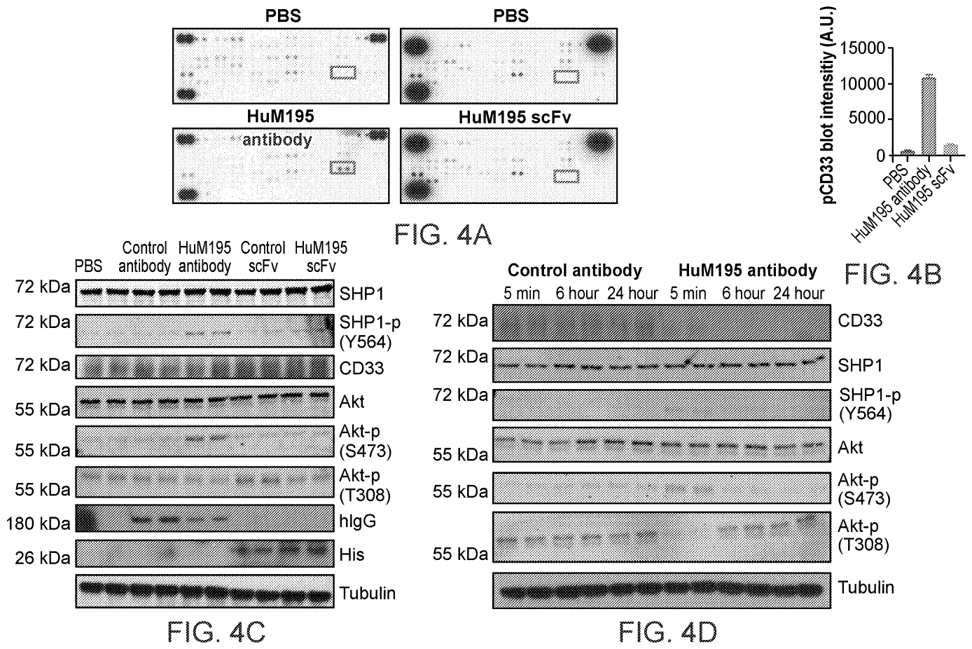

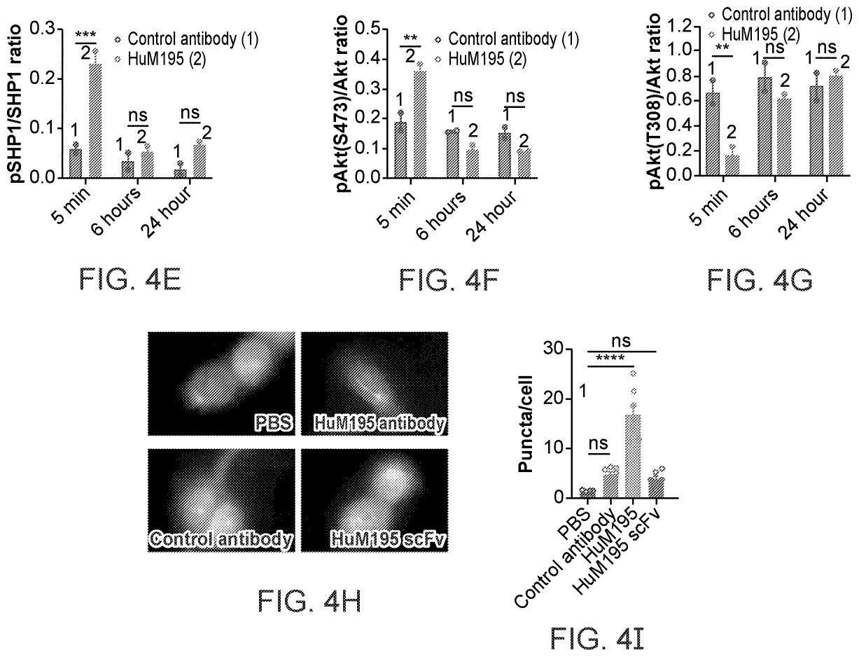

The cytoplasmic domain of CD33 contains an ITIM that upon activation and phosphorylation presumably inhibits activation of microglia. We set to investigate the downstream signaling of CD33 by HuM195 using a human phospho-immunoreceptor array, which detected over 50 potential phospho-immunoreceptor including CD33. We treated THP-1 cells with PBS control, HuM195 antibody or scFv (1 μg/ml) for 1 min. Although the assay can detect large numbers of immune-related phospho-proteins, we were not able to detect significant changes in most of the tested proteins. However, CD33 phosphorylation was detected only after treatment with full length antibody HuM195, whereas HuM195 scFv treatment did not lead to CD33 phosphorylation (FIGS. 4A-4B). Examination of the downstream phosphorylation of SHP1 revealed that the brief treatment with HuM195 antibody led to an increase in SHP1 phosphorylation at Y564 (FIG. 4C); this was not seen with the HuM195 scFv or the relevant controls. There are number of potential downstream phosphorylation events derived from CD33 signaling, either through PI3K/AKT or MAPK. We were able to detect modification in AKT phosphorylation with the HuM195 antibody treatment. Specifically, we observed an increase in p-AKT (S473) and decrease in p-AKT (T308) (FIG. 4C). No changes in PI3K or MAPK phosphorylation were detected. Since CD33 is classified as an inhibitory protein by its ITIM, the phosphorylation signaling induced by HuM195 antibody can potentially result in inhibition of microglial activation. However, we have shown that HuM195 also promotes the degradation of CD33, thereby eliminating its signaling entirely. To test the time course of HuM195 antibody induced CD33 signaling, we treated THP-1 cells for 5 min, 6 and 24 hours and perform western blot analysis. Activation of CD33 phosphorylation signaling by HuM195 antibody is transient and correlated with the presence of CD33 (FIG. 4D). Both p-SHP1 (FIG. 4E) and p-AKT (S473) (FIG. 4F) increases while p-AKT (T308) (FIG. 4G) decreases after 5 min of HuM195 treatment compared to control antibody, as expected. However, after 6- or 24-hours treatment with HuM195, no dateable CD33 was seen and the phosphorylation of either SHP1 or AKT returned to its baseline as compared to the control antibody treatment.

Although, both HuM195 antibody and scFv were able to increase Aβ clearance by CD33 degradation, there was an apparent difference in the CD33 activation and phosphorylation between the full-length antibody and scFv treatments. Dimerization of cell surface receptor occurs frequently and is thought to initiate cell signaling (19). To test whether HuM195 antibody and scFv promoted CD33 dimerization, we utilized the proximity ligation assay (PLA) by direct DNA labeling of commercial αCD33 primary antibody to a plus or minus strand. Treatment with HuM195 antibody in HL60 cells, which highly express CD33, displayed a significantly higher PLA spot signal compared to HuM195 scFv or antibody control (FIGS. 411-4I). Collectively, these data indicated that HuM195 antibody, but not it corresponding scFv, activates CD33 and initiates ITIM phosphorylation downstream signaling cascade through receptor dimerization. This HuM195 mediated CD33 phosphorylation signaling quickly disappeared following CD33 degradation. Nevertheless, treatment by either HuM195 antibody or HuM195 scFv induced CD33 internalization and degradation and consequently increased phagocytosis by eliminating the inhibitory signal of CD33.

These results demonstrate that the anti-CD33 immunoglobulin-related compositions of the present technology are useful in methods for treating or preventing Alzheimer's disease and/or reducing brain accumulation and/or persistence of Aβ plaques in a subject in need thereof.

### Example 4: Treatment with HuM195 Antibody Induces Expression of IL33 and Increases Aβ42 Uptake

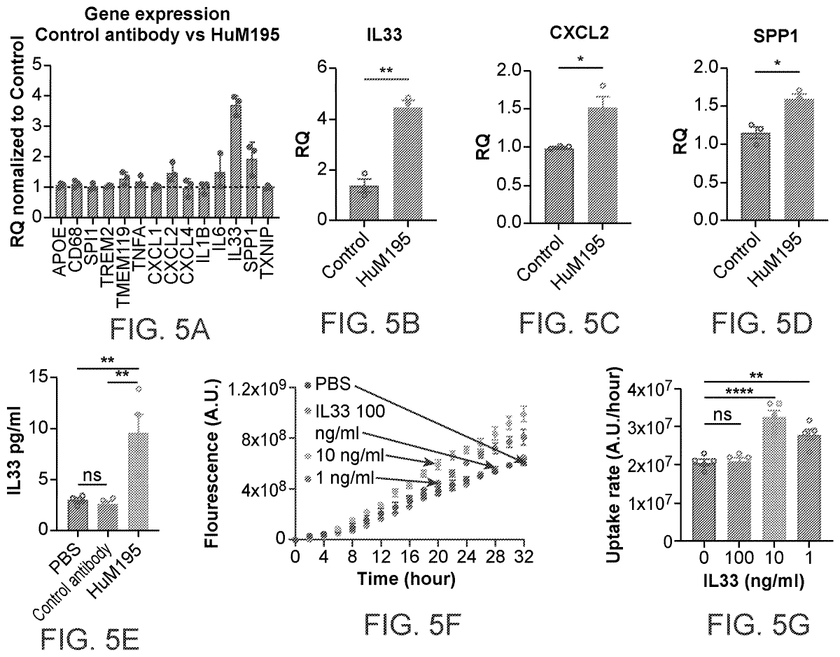

Evidence of the therapeutic potential of targeting CD33 in AD was demonstrated by the knockout of CD33 in AD models in which a reduction in Aβ plaque burden was seen (4,5). In addition, RNA-seq profiling of microglia from CD33 knockout cells showed upregulation of neuroinflammatory genes (5). Reduction of CD33 may lead to modification in the inflammatory response to further increase uptake and clearance of Aβ. To examine the effect of HuM195 on the inflammatory response, we treated THP-1 cells for 24 hours and performed qPCR assay on a panel of inflammatory genes observed in the CD33 knockout microglia (5). Compared to the control antibody treatment (FIG. 5A), the most significant increase in mRNA levels were IL33 (FIG. 5B) by ˜4 fold, CXCL2 (FIG. 5C) by ˜1.5 fold and SPP1 (FIG. 5D) by ˜1.5 fold. Moreover, HuM195 antibody treatment resulted in an increase secretion of IL33 into the media (FIG. 5E), confirming not only the gene expression increase, but also expression and secretion of the cytokine. Interestingly, IL33 was implicated in the reprogramming of microglia to an activated state increasing its Aβ clearance capability (20). Therefore, we asked whether IL33 alone could enhance Aβ uptake in our model systems. Activated THP1 cells were treated with increase concentrations of human IL33 for 4 hours followed by the addition of pHrodo-Aβ42 and live imaging as previously described for the antibody modulation experiments. We observed an increase in fluorescence with IL33 treatment (10 ng/ml and 1 ng/ml) (FIG. 5F) and a significantly increase in uptake rate compared to the vehicle control (FIG. 5G). These data demonstrate that degradation of CD33 by HuM195 treatment also results in secretion of the IL33 cytokine and CXCL2 chemokine which in turn may further recruit and activate cells to enhance Aβ uptake and clearance.

These results demonstrate that the anti-CD33 immunoglobulin-related compositions of the present technology are useful in methods for treating or preventing Alzheimer's disease and/or reducing brain accumulation and/or persistence of Aβ plaques in a subject in need thereof.

### Example 5: Cells Secreting Hu195 scFv Enhances Phagocytosis of Aβ42 by Human Macrophages

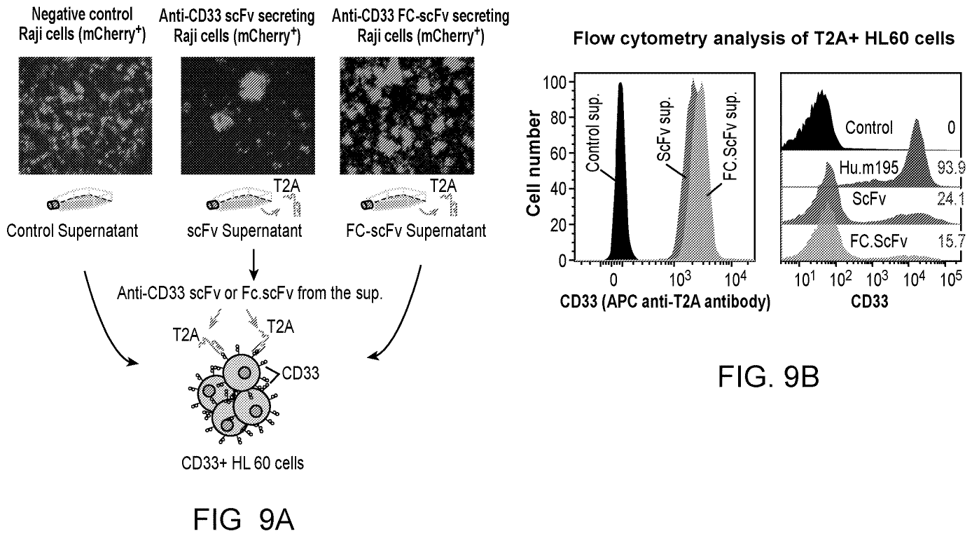

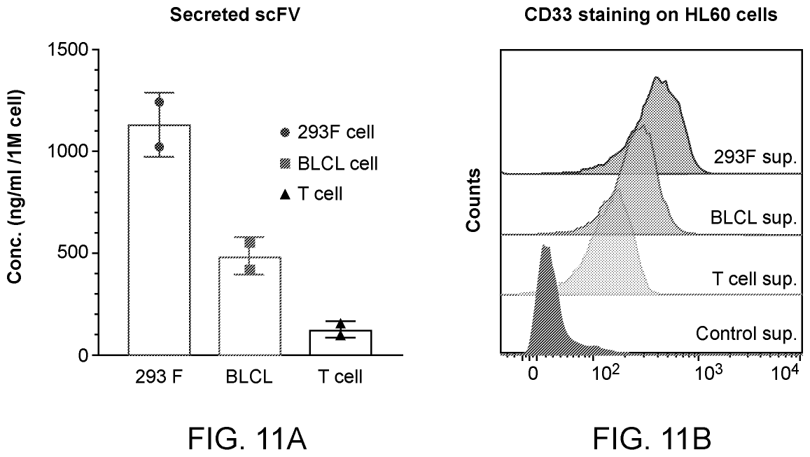

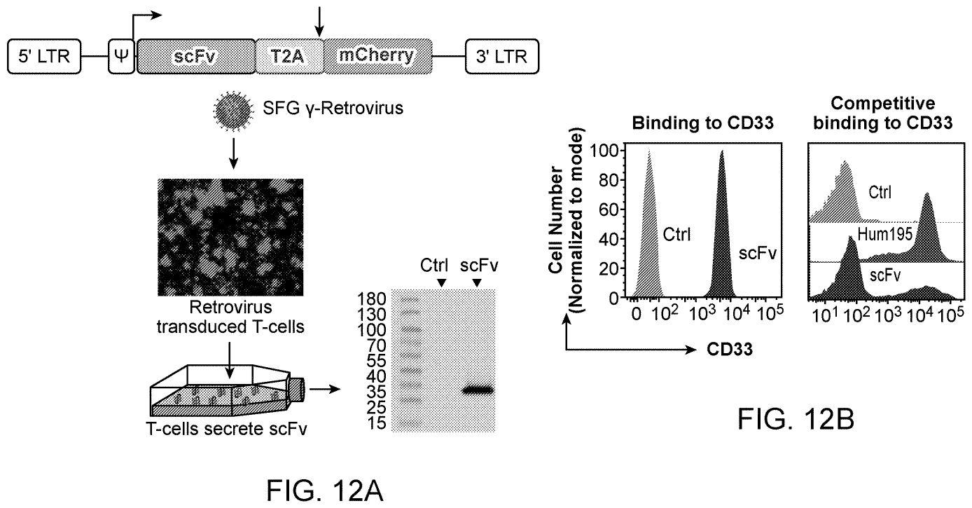

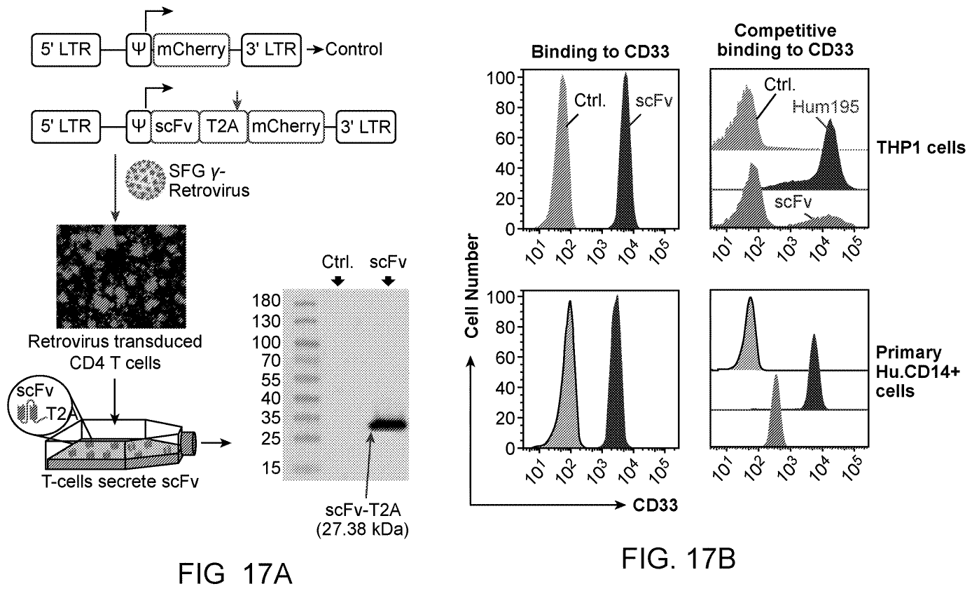

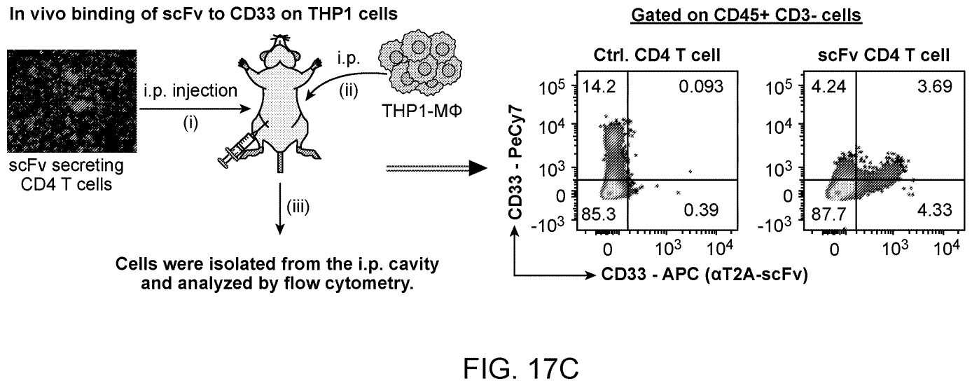

As shown in FIGS. 9A-9B, engineered B-cells secrete anti-CD33 scFv and FC.scFv which recognize CD33 antigen on HL60 leukemia cells. Engineered B cells and T cells comprising Hu195 scFv nucleic acids successfully secreted anti-CD33 scFv, as evidenced by Hu195 scFv binding to CD33 antigen on HL60 leukemia cells. FIGS. 11A-11B. Anti-CD33 scFv secreting CD4 T-cells were constructed and their binding to CD33 antigen was characterized in accordance with FIGS. 12A-12B and 17A-17C.

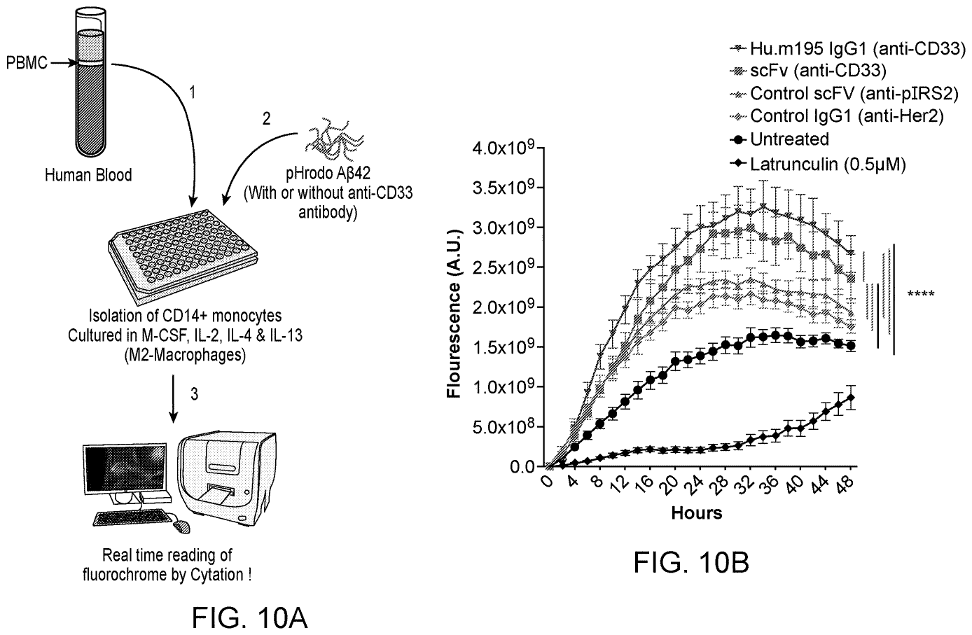

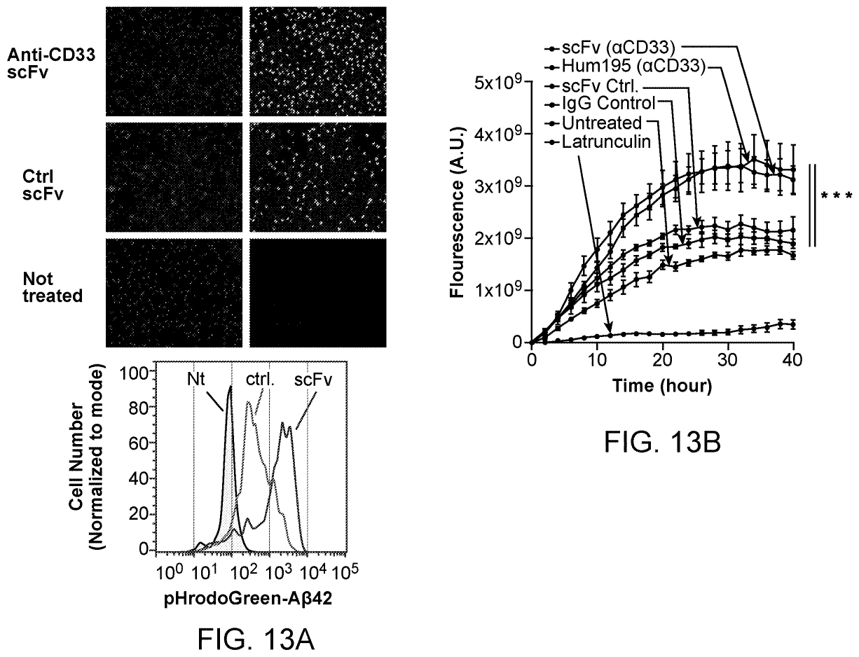

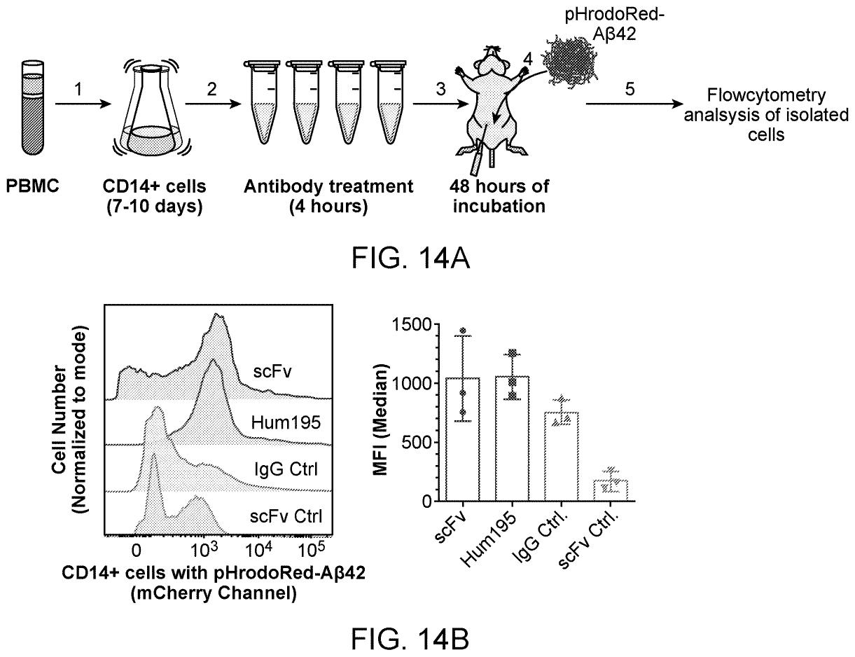

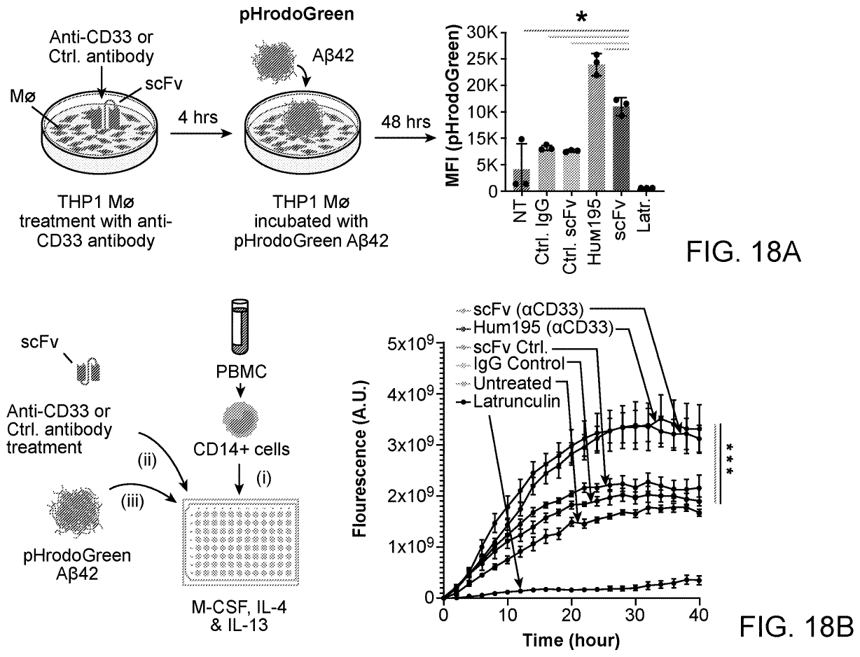

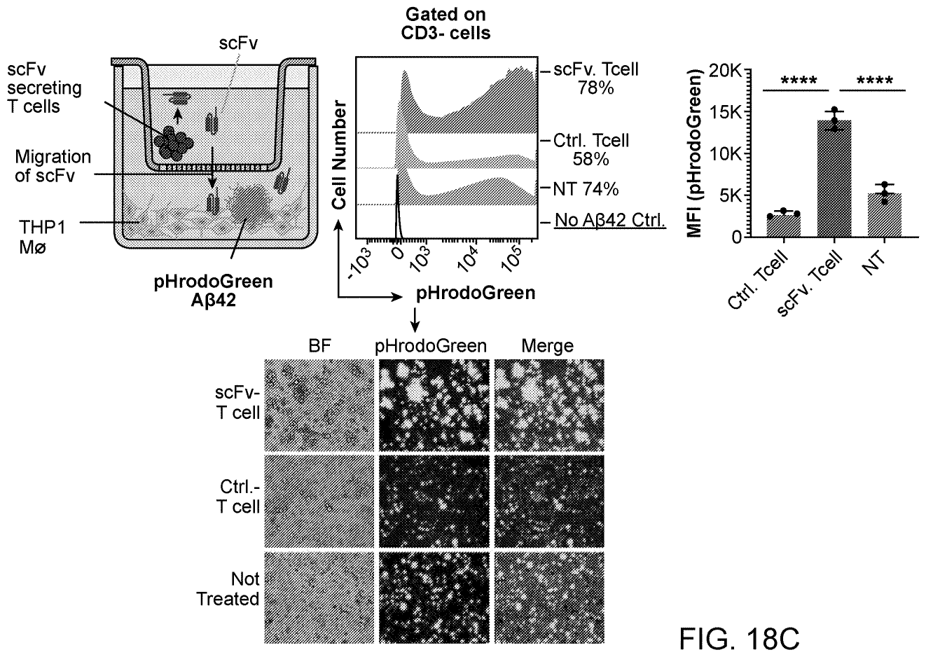

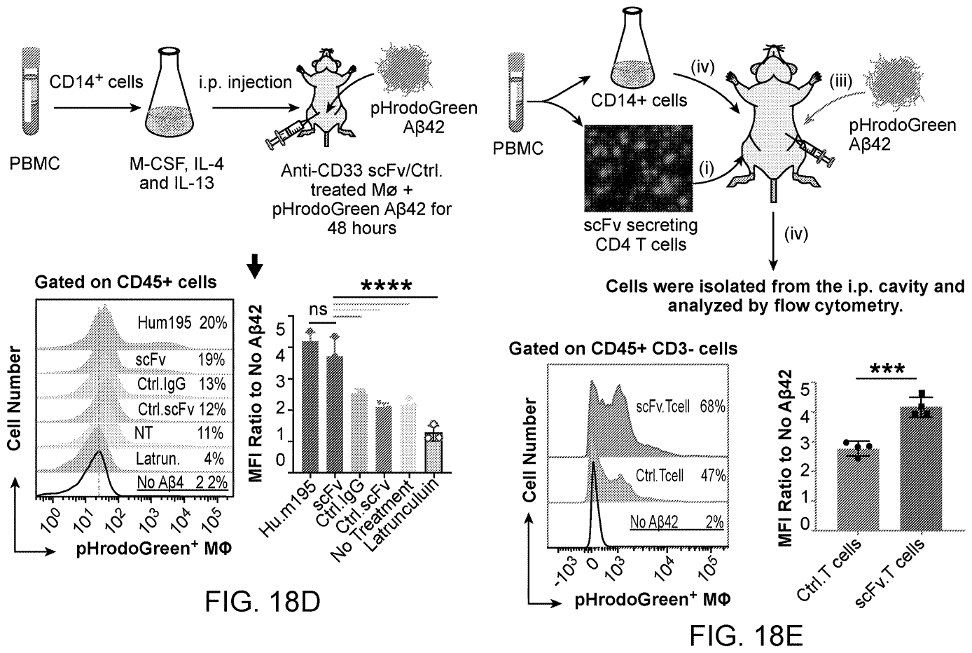

The engineered cell-secreted anti-CD33 scFv enhanced phagocytosis of Aβ42 by human macrophages in vitro (FIGS. 10A-10B, FIGS. 13A-13B) and in vivo (FIGS. 14A-14B and FIGS. 18A-18E).

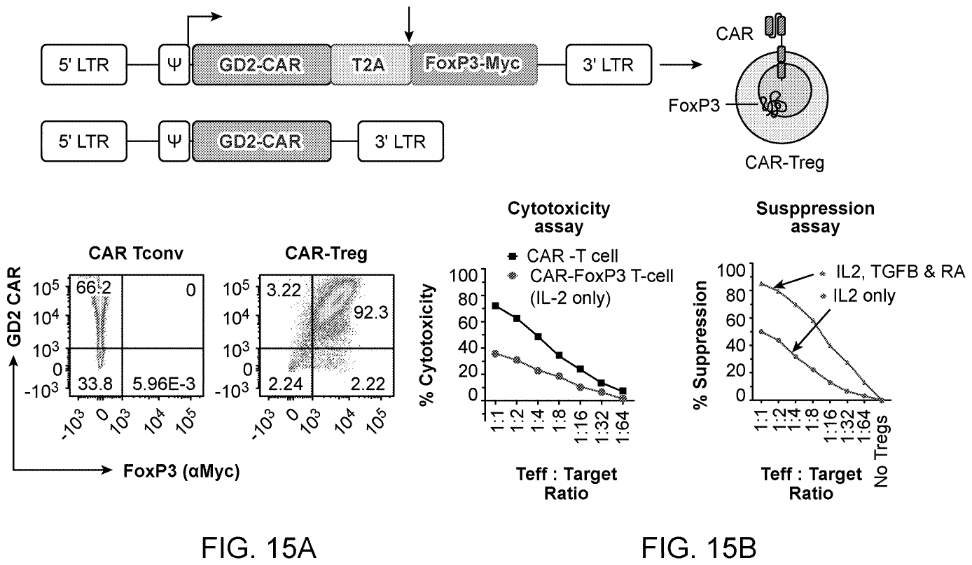

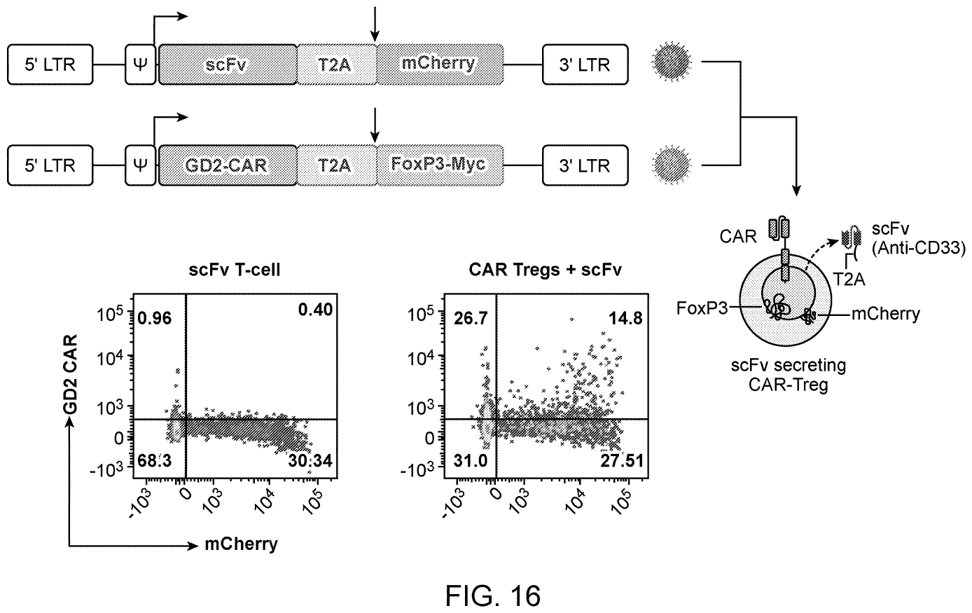

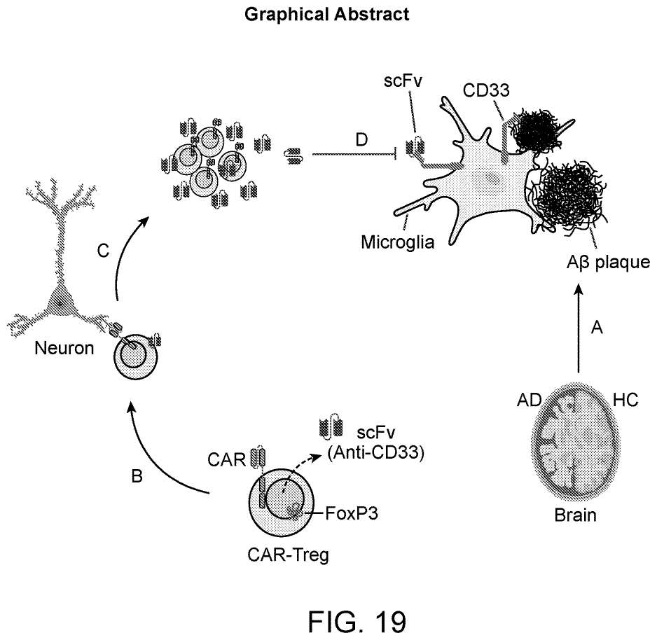

FIGS. 19A-19D show a representation of CAR Treg therapy for Aβ clearance in the AD brain. FIG. 19A: Extracellular deposition of Aβ plaques in the brain leads to an early toxic event in the pathogenesis of AD. Microglia and brain resident macrophages are crucial in clearing the early Aβ fibrils by phagocytosis. However, Aβ fibrils inhibit microglial phagocytosis via engaging with their inhibitory receptor CD33 and other molecules. In addition, Aβ fibril deposition induces the production of pro-inflammatory cytokines by microglia, which contribute to neurodegeneration. We have genetically engineered Tregs to express a neuronal antigen-specific CAR and secrete anti-CD33 scFv antibodies (FIGS. 15A-15B, 16 and 19B).

These CAR-Tregs are anticipated to proliferate in response to neuronal antigen in the brain, maintaining an anti-inflammatory environment, and secrete scFv antibody locally (FIG. 19C). As shown in FIG. 19D, CAR Tregs could allow active Aβ phagocytosis by microglia and brain resident macrophages via blocking their inhibitory CD33 receptor via secreted anti-CD33 scFv antibody.

These results demonstrate that the anti-CD33 immunoglobulin-related compositions of the present technology are useful in methods for treating or preventing Alzheimer's disease and/or reducing brain accumulation and/or persistence of Aβ plaques in a subject in need thereof.

### Example 6: Therapeutic Effects of Hu195 Immunoglobulin-Related Compositions in an In Vivo Alzheimer Disease Model

5×FAD mice (Jackson Laboratory) express human APP and PSEN1 transgenes with a total of five AD-linked mutations: the Swedish (K670N/M671L), Florida (I716V), and London (V717I) mutations in APP, and the M146L and L286V mutations in PSEN1.

Because HuM195 specifically targets human CD33, the ability to test HuM195's therapeutic effect in mouse models is limited. To this end, 5×FAD and 5×FAD; CD33^{-/-} with humanized CD33 mice are treated with different concentrations of Hu195 antibody or Hu195 scFv via intravenous infusion or via subcutaneous injections.

Thioflavine S staining: Brains from 4-month-old perfused 5×FAD (Jackson Laboratory) and 5×FAD/humanized CD33 mice are extracted and post-fixed overnight in 4% PFA at 4° C. Brains are then washed 3 times in PFA and incubated in 30% sucrose solution overnight at 4° C. Brains are then flash frozen in Oct compound and stored at −80° C. Samples are serially sectioned coronally at 30 μm and stored at −20° C. in tissue storage solution (30% sucrose, 30% ethylene glycol in 0.1M phosphate buffer). Every 6th section is stained with thioflavine-S (1% in ddH₂O for 8 minutes) and washed 3 times in PBS with a fourth wash overnight at 4° C. (samples are protected from light at all times). Stained sections are mounted on charged slides and imaged.

Ethovision video tracking. Ethovision XT video tracking software (Noldus) is used to track mice in all behaviour experiments. Mice are detected by color contrast compared to background and defined by three points, a nose point, body point and tail point so that direction of the mouse body can be assessed.

Activity detection. Pixels changed between each frame of video (12.5 frames/s) are recorded. A threshold is set whereby a specific number of pixels changed constitutes activity. The threshold is set by visualizing mice within the chamber so that when the mouse is completely inactive, there is no activity and when the mouse moves there is activity.

Y Maze. The Y-maze has three arms of equal length that extend from a central platform at a 120° angle. The animal is released in the centre of the maze and allowed to freely explore the three arms (A, B, C) for 5 minutes. Over the trial, the mouse is expected to show a preference to enter the less recently visited arm (A>B>C>A> . . . ). The order of arm entrances is recorded in order to calculate the percentage of correct cycles. An arm entry is scored when all four limbs are within the arm. A cycle is defined as entries into the three different arms without revisiting an arm prior to visiting the other two. The maximum number of cycles is equal to total number of visits minus 2. Percentage of correct cycles=number of cycles/(#of entrances−2)×100. Novel Object Recognition. Mice are first placed in an open field to habituate and are then returned to their home cage. In the first trial, mice are placed in the open field containing two identical red cube objects for 2 minutes and then returned to their home cage. In the second trial, one of the red cubes is replaced with a blue cylinder and then mice are placed back in the open field for 2 minutes. During both trials the amount of time spent investigating each object (nose within 3 cm of the object) is recorded. In the first trial, the object to be replaced with the novel object is defined as object 1, in the second trial the novel object is defined as object 1. A differential index (DI) is calculated for both trials as (time with object 1−times with object 2)/time with both objects, therefore a DI of 1 indicates total preference for the novel object, a DI of 0 represents no preference and a DI of −1 indicates total preference for the familial object.

Activity tracking. Mice are placed in a 17×17×25(h)cm cage (ugo basile) for 90 seconds and activity detection is used to determine the percent of time sent activity.

Behavioral Tests. For the open field exploration test, mice are placed in the center of a dimly lit chamber of an open field arena. Mouse movements are recorded and tracked by the automatic video tracking system EthoVision XT (Noldus) for 15 minutes. The area of the arena is virtually divided into a center zone (Pasquarella et al., Sci. Total Environ. 2012; 420: 289-299) and four corner squares. Total distance traveled, the time spent in each area, and horizontal and vertical activity are monitored. The Morris water maze test is performed with minor adjustment as previously described (Vorhees and Williams, Nat. Protoc. 2006; 1: 848-858). Spatial memory testing is conducted in a circular tank (diameter 1.22 m) filled with opacified water at 23° C. The water tank is dimly lit and surrounded by a white curtain. The maze is virtually divided into four quadrants, with one containing a hidden platform (diameter 10 cm) that is submerged 0.5 cm below the water level. Four prominent cues are placed outside the maze as spatial references. Mice are placed in the water facing the tank wall at different start positions across trials in a quasi-random fashion to prevent strategy learning. Mice are allowed to search for the platform for 1 minute; if the mice do not find the platform, they are guided toward it where they remain for 20 s. Each mouse goes through four trials (one from each start position) per day for seven consecutive days. After each trial, the mouse is dried and placed back into its cage until the start of the next trial. All mouse movements are recorded by the computerized tracking system EthoVision XT (Noldus) that calculates distances moved and time required to reach the platform (escape latency), along with swim speed. The spatial probe trial is conducted 24 hours after the last training session (on day 8). For the probe trial, the platform is removed and mice are allowed to swim for 1 minute. The time spent by the mice in the area surrounding the location where the platform used to be (platform plus) is recorded. The platform plus surrounding the target is larger than the target itself, but smaller than the target quadrant. Data is calculated as time in the platform plus/60 s*l100% and is given in percentage. The number of times the mice crossed the platform is also shown. On days 9 and 10, the visual cued testing is conducted, where the platform is flagged and placed above the water surface. Mice are allowed to swim to the visible platform for 1 minute and each mouse performed two trials per day. Time required to reach the visible platform is shown.

Aβ ELISA. Mouse cortices are homogenized in 5 volumes of tissue homogenization buffer [25 mM Tris-HCl at pH 7.4, 130 mM NaCl, 2.7 mM KCl, 5 mM EDTA, phosphatase inhibitor (Thermo Fisher Scientific), EDTA-free protease inhibitor cocktail (Roche) and 2 mM 1,10-phenantroline (Sigma Aldrich)], using a Polytron benchtop lab homogenizer (Wheaton) at 4° C. The homogenates are centrifuged at 100,000 g for 1 hour at 4° C. using an Optima TL ultracentrifuge and a TLA 120.2 rotor (Beckman Coulter). Supernatants are collected and used to measure TBS-soluble Aβ. The pellet is extracted in 70% formic acid (equal volume of TBS) with a hand homogenizer (Wheaton) on ice. Samples are centrifuged at 100,000 g for 1 hour at 4° C. and supernatants are collected. Formic acid supernatants are neutralized with 1M Tris-base, pH 11 (1:17 v:v) and samples are used to measure formic acid-soluble Aβ. TBS-soluble and formic acid-soluble Aβ are measured by sandwich ELISA using commercially available kits (Wako). The capture antibody is mouse monoclonal anti-human Aβ antibody (clone BNT77). HRP-conjugated monoclonal anti-human A040 (clone BA27) and monoclonal anti-human Aβ42 (clone BC05) antibodies are used for detection of A040 and Aβ42, respectively.

Immunohistochemistry end image analysis. Mice are deeply anesthetized with CO₂ and transcardially perfused with 0.9% sodium chloride. The brains are quickly removed from the skull. The right brain hemisphere is submerged in ice-cold 0.9% sodium chloride. Cortex and hippocampus are dissected and snap frozen in dry ice. Samples are stored at −80° C. until further processing. The left hemisphere is fixed in 4% paraformaldehyde for 72 hours at 4° C. Subsequently, the left hemisphere is dehydrated with ethanol and embedded in paraffin. Paraffin-embedded tissue is cut into 6 μm-thick coronal sections using a paraffin microtome, transferred onto microscope slides and stored at room temperature.

Coronal sections are deparaffinized and incubated with 3% H₂O₂ to quench endogenous peroxidases for DAB staining. Antigen retrieval is performed using Diva Decloaker (Biocare Medical) or citrate buffer (0.01M, pH 6.0, 0.05% Tween-20) in a microwave oven (95° C., 20 minutes). For immunostaining with the anti-Aβ antibody (clone 3D6 directed against Aβ residues 1-5), sections are incubated in 90% formic acid (Sigma Aldrich) for 5 minutes, after antigen retrieval. Sections are subsequently blocked using 2% BSA, 0.1% Triton X-100 in PBS, or alternatively with Antibody diluent (Cell Signaling). The following primary antibodies are added overnight at 4° C.: anti-Aβ (mouse monoclonal, clone 3D6, 1:2500, Elan Pharmaceuticals), anti-cleaved Caspase-3 (rabbit monoclonal, 1:500, Cell Signaling), anti-Iba1 (rabbit polyclonal, 1:500, Wako), anti-Iba1 (goat polyclonal, 1:1000, Novus Biologicals), and anti-P2ry12 (rabbit polyclonal, 1:2000, AnaSpec). The primary antibody targeting NeuN (mouse monoclonal, 1:300, Millipore Sigma) is added for 1 hour at room temperature. Primary antibodies are detected using appropriate biotinylated secondary antibodies (1:300) and VECTASTAIN Elite ABC HRP kits (Vector Laboratories) and developed with DAB (Sigma Aldrich) according to the provider's instructions. To visualize dense core Aβ plaques, sections are stained with Congo red (Sigma Aldrich). To quantify the number of cells, sections are stained with hematoxylin and eosin (Vector Laboratories) following manufacturer's directions. Sections are dehydrated with increasing concentrations of ethanol, cleared with xylene, and coverslipped with xylene-based mounting medium (Cytoseal XYL, Thermo Fisher Scientific). Sections are imaged with the Nikon Eclipse Ci microscope that is equipped with a DS-Ri2 camera and NIS-Elements Advanced Research imaging software (Nikon, Tokyo, Japan).

For immunofluorescence experiments, primary antibodies are detected with species-specific Alexa Fluor 488/568-coupled secondary antibodies (1:500, Thermo Fisher Scientific). Sections are mounted with aqueous mounting medium containing DAPI and anti-fading reagent (ProLong Gold Antifade Mountant, Invitrogen). Stained sections are analyzed by fluorescence confocal microscopy on a Nikon C2si laser scanning microscope that is equipped with NIS-Elements Advanced Research software (Nikon, Tokyo, Japan).

For the assessment of Aβ plaque burden, four coronal sections spanning the cortex and hippocampus (at different depths on the rostro-caudal axis) are imaged for each animal. The amyloid plaque burden (area occupied by all plaques divided by the total area) is estimated in the cortex and hippocampus for each section using the Analyze Particles plugin of ImageJ software (NIH). Values from each section are averaged to generate a mean plaque burden for each animal.

For quantification of neuronal cell density, the cortical layer 5 and the CA1 area in the hippocampus are imaged at ×10 and ×20 magnifications, respectively. Two coronal sections spanning the cortex and hippocampus at different depths on the rostro-caudal axis are analyzed for each animal. Three images are acquired on matching areas of each the cortex and hippocampus per section. The number of neuronal cells (using NeuN labeling) is determined using the Cell Counter plugin of ImageJ and is divided by the area occupied by the cells. Values from each section are averaged to obtain a mean neuronal cell density for each animal.

For quantification of Caspase-3⁺ cell density, the cortex and hippocampus are imaged at ×20 magnification. Two coronal sections spanning the cortex and hippocampus at different depths on the rostro-caudal axis are analyzed for each animal. Five to six images are acquired on matching areas of each the cortex and hippocampus per section. The number of Caspase-3⁺ cells is determined using the Cell Counter plugin of ImageJ and is divided by the area occupied by the cells. Values from each section are averaged to obtain a mean Caspase-3⁺ cell density for each animal.

To analyze the Iba1⁺ cell response to Aβ plaques, coronal sections are stained with Iba1 and 3D6 for visualization of plaques, as described above. Three coronal sections spanning the cortex and hippocampus at different depths are analyzed for each animal. Images are acquired in random regions of the cortices and hippocampi at ×20 magnification with the Nikon C2 confocal microscope. The number of Iba1+ cells is determined using the Analyze Particles plugin of ImageJ and is divided by the area occupied by the cells. Values from each image are averaged to obtain a mean Iba1⁺ cell density for each animal. For individual Aβ plaque analysis, images are acquired at ×40 magnification and approximately 25 plaques are randomly selected and analyzed for each animal. The number of Iba1+ cells is quantified within a 20 m distance from the plaque by using the Cell Counter plugin of ImageJ. The distance between Iba1⁺ cells and the center of their associated plaques is calculated using the Measure function of ImageJ. Surface area of the Iba1⁺ cell body is measured using the Surface function of Imaris software (Bitplane). Area of the plaque is measured in the 3D6 channel using the Analyze Particles function of ImageJ.

To analyze the colocalization between P2ry12+ and Iba1+ cells, coronal sections are stained with the anti-P2ry12 (red) and anti-Iba1 (green) antibodies, as described above. Two coronal sections spanning the cortex and hippocampus at different depths are analyzed for each animal. Five to six images are acquired on matching areas of each the cortex and hippocampus per section at ×20 magnification with the Nikon C2 confocal microscope. The number of P2ry12+Iba1+ cells is determined using the Analyze Particles function of ImageJ and is divided by the area occupied by the cells. Values from each image are averaged to obtain a mean P2ry12+Iba1⁺ cell density for each animal. To determine the % P2ry12+Iba11/Iba1+ cells, the number of P2ry12+Iba1+ cells is divided by the total number of Iba1+ cells and multiplied by 100%.

It is anticipated that 5×FAD and/or 5×FAD/humanized CD33 mice treated with Hu195 antibody or Hu195 scFv will show reduced Aβ pathology and/or reduced cognitive or behavioral abnormalities compared to untreated controls.

These results demonstrate that the anti-CD33 immunoglobulin-related compositions of the present technology are useful in methods for treating or preventing Alzheimer's disease and/or reducing brain accumulation and/or persistence of Aβ plaques in a subject in need thereof.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1. Hardy, J. A., and Higgins, G. A. (1992) Alzheimer's disease: the amyloid cascade hypothesis. Science 256, 184-185
2. Efthymiou, A. G., and Goate, A. M. (2017) Late onset Alzheimer's disease genetics implicates microglial pathways in disease risk. Mol Neurodegener 12, 43
3. Villegas-Llerena, C., Phillips, A., Garcia-Reitboeck, P., Hardy, J., and Pocock, J. M. (2016) Microglial genes regulating neuroinflammation in the progression of Alzheimer's disease. Curr Opin Neurobiol 36, 74-81
4. Griciuc, A., Serrano-Pozo, A., Parrado, A. R., Lesinski, A. N., Asselin, C. N., Mullin, K., Hooli, B., Choi, S. H., Hyman, B. T., and Tanzi, R. E. (2013) Alzheimer's disease risk gene CD33 inhibits microglial uptake of amyloid beta. Neuron 78, 631-643
5. Griciuc, A., Patel, S., Federico, A. N., Choi, S. H., Innes, B. J., Oram, M. K., Cereghetti, G., McGinty, D., Anselmo, A., Sadreyev, R. I., Hickman, S. E., El Khoury, J., Colonna, M., and Tanzi, R. E. (2019) TREM2 Acts Downstream of CD33 in Modulating Microglial Pathology in Alzheimer's Disease. Neuron 103, 820-835 e827
6. Karch, C. M., Jeng, A. T., Nowotny, P., Cady, J., Cruchaga, C., and Goate, A. M. (2012) Expression of novel Alzheimer's disease risk genes in control and Alzheimer's disease brains. PLoS One 7, e50976
7. Garnache-Ottou, F., Chaperot, L., Biichle, S., Ferrand, C., Remy-Martin, J. P., Deconinck, E., de Tailly, P. D., Bulabois, B., Poulet, J., Kuhlein, E., Jacob, M. C., Salaun, V., Arock, M., Drenou, B., Schillinger, F., Seilles, E., Tiberghien, P., Bensa, J. C., Plumas, J., and Saas, P. (2005) Expression of the myeloid-associated marker CD33 is not an exclusive factor for leukemic plasmacytoid dendritic cells. Blood 105, 1256-1264
8. Zhao, L. (2019) CD33 in Alzheimer's Disease—Biology, Pathogenesis, and Therapeutics: A Mini-Review. Gerontology 65, 323-331
9. Sutherland, M. K., Yu, C., Lewis, T. S., Miyamoto, J. B., Morris-Tilden, C. A., Jonas, M., Sutherland, J., Nesterova, A., Gerber, H. P., Sievers, E. L., Grewal, I. S., and Law, C. L. (2009) Anti-leukemic activity oflintuzumab (SGN-33) in preclinical models of acute myeloid leukemia. MAbs 1, 481-490
10. Kasim, J. K., Kavianinia, I., Harris, P. W. R., and Brimble, M. A. (2019) Three Decades of Amyloid Beta Synthesis: Challenges and Advances. Front Chem 7, 472
11. Kotler, S. A., Walsh, P., Brender, J. R., and Ramamoorthy, A. (2014) Differences between amyloid-beta aggregation in solution and on the membrane: insights into elucidation of the mechanistic details of Alzheimer's disease. Chem Soc Rev 43, 6692-6700
12. Dolman, N. J., Kilgore, J. A., and Davidson, M. W. (2013) A review of reagents for fluorescence microscopy of cellular compartments and structures, part I: BacMam labeling and reagents for vesicular structures. Curr Protoc Cytom Chapter 12, Unit 12 30
13. Abbott, N. J., Patabendige, A. A., Dolman, D. E., Yusof, S. R., and Begley, D. J. (2010) Structure and function of the blood-brain barrier. Neurobiol Dis 37, 13-25
14. Louveau, A., Harris, T. H., and Kipnis, J. (2015) Revisiting the Mechanisms of CNS Immune Privilege. Trends Immunol 36, 569-577
15. Poduslo, J. F., Curran, G. L., and Berg, C. T. (1994) Macromolecular permeability across the blood-nerve and blood-brain barriers. Proc Natl Acad Sci USA 91, 5705-5709
16. Ahmad, Z. A., Yeap, S. K., Ali, A. M., Ho, W. Y., Alitheen, N. B., and Hamid, M. (2012) scFv antibody: principles and clinical application. Clin Dev Immunol 2012, 980250
17. Ritchie, M., Tchistiakova, L., and Scott, N. (2013) Implications of receptor-mediated endocytosis and intracellular trafficking dynamics in the development of antibody drug conjugates. MAbs 5, 13-21
18. Hagemann, U. B., Wickstroem, K., Wang, E., Shea, A. O., Sponheim, K., Karlsson, J., Bjerke, R. M., Ryan, O. B., and Cuthbertson, A. S. (2016) In Vitro and In Vivo Efficacy of a Novel CD33-Targeted Thorium-227 Conjugate for the Treatment of Acute Myeloid Leukemia. Mol Cancer Ther 15, 2422-2431
19. Weiss, A., and Schlessinger, J. (1998) Switching signals on or off by receptor dimerization. Cell 94, 277-280
20. Lau, S. F., Chen, C., Fu, W. Y., Qu, J. Y., Cheung, T. H., Fu, A. K. Y., and Ip, N. Y. (2020) IL-33-PU.1 Transcriptome Reprogramming Drives Functional State Transition and Clearance Activity of Microglia in Alzheimer's Disease. Cell Rep 31, 107530
21. Perez-Oliva, A. B., Martinez-Esparza, M., Vicente-Fernandez, J. J., Corral-San Miguel, R., Garcia-Penarrubia, P., and Hernandez-Caselles, T. (2011) Epitope mapping, expression and post-translational modifications oftwo isoforms of CD33 (CD33M and CD33m) on lymphoid and myeloid human cells. Glycobiology 21, 757-770
22. Malik, M., Simpson, J. F., Parikh, I., Wilfred, B. R., Fardo, D. W., Nelson, P. T., and Estus, S. (2013) CD33 Alzheimer's risk-altering polymorphism, CD33 expression, and exon 2 splicing. J Neurosci 33, 13320-13325
23. Bhattacherjee, A., Jung, J., Zia, S., Ho, M., Eskandari-Sedighi, G., St Laurent, C. D., McCord, K. A., Bains, A., Sidhu, G., Sarkar, S., Plemel, J. R., and Macauley, M. S. (2021) The CD33 short isoform is again-of-function variant that enhances Abetal-42 phagocytosis in microglia. Mol Neurodegener 16, 19
24. Bradshaw, E. M., Chibnik, L. B., Keenan, B. T., Ottoboni, L., Raj, T., Tang, A., Rosenkrantz, L. L., Imboywa, S., Lee, M., Von Korff, A., Alzheimer Disease Neuroimaging, I., Morris, M. C., Evans, D. A., Johnson, K., Sperling, R. A., Schneider, J. A., Bennett, D. A., and De Jager, P. L. (2013) CD33 Alzheimer's disease locus: altered monocyte function and amyloid biology. Nat Neurosci 16, 848-850
25. Griciuc, A., Federico, A. N., Natasan, J., Forte, A. M., McGinty, D., Nguyen, H., Volak, A., LeRoy, S., Gandhi, S., Lerner, E. P., Hudry, E., Tanzi, R. E., and Maguire, C. A. (2020) Gene therapy for Alzheimer's disease targeting CD33 reduces amyloid beta accumulation and neuroinflammation. Hum Mol Genet 29, 2920-2935
26. Bhattacherjee, A., Rodrigues, E., Jung, J., Luzentales-Simpson, M., Enterina, J. R., Galleguillos, D., St Laurent, C. D., Nakhaei-Nejad, M., Fuchsberger, F. F., Streith, L., Wang, Q., Kawasaki, N., Duan, S., Bains, A., Paulson, J. C., Rademacher, C., Giuliani, F., Sipione, S., and Macauley, M. S. (2019) Repression of phagocytosis by human CD33 is not conserved with mouse CD33. Commun Biol 2, 450
27. Scheinberg, D. A., Tanimoto, M., McKenzie, S., Strife, A., Old, L. J., and Clarkson, B. D. (1989) Monoclonal antibody M195: a diagnostic marker for acute myelogenous leukemia. Leukemia 3, 440-445
28. Jilani, I., Estey, E., Huh, Y., Joe, Y., Manshouri, T., Yared, M., Giles, F., Kantarjian, H., Cortes, J., Thomas, D., Keating, M., Freireich, E., and Albitar, M. (2002) Differences in CD33 intensity between various myeloid neoplasms. Am J Clin Pathol 118, 560-566
29. Maakaron, J. E., Rogosheske, J., Long, M., Bachanova, V., and Mims, A. S. (2021) CD33-Targeted Therapies: Beating the Disease or Beaten to Death? J Clin Pharmacol 61, 7-17
30. Caron, P. C., Co, M. S., Bull, M. K., Avdalovic, N. M., Queen, C., and Scheinberg, D. A. (1992) Biological and immunological features of humanized M195 (anti-CD33) monoclonal antibodies. Cancer Res 52, 6761-6767
31. Laing, A. A., Harrison, C. J., Gibson, B. E. S., and Keeshan, K. (2017) Unlocking the potential of anti-CD33 therapy in adult and childhood acute myeloid leukemia. Exp Hematol 54, 40-50
32. Walter, R. B., Appelbaum, F. R., Estey, E. H., and Bernstein, I. D. (2012) Acute myeloid leukemia stem cells and CD33-targeted immunotherapy. Blood 119, 6198-6208
33. Laszlo, G. S., Estey, E. H., and Walter, R. B. (2014) The past and future of CD33 as therapeutic target in acute myeloid leukemia. Blood Rev 28, 143-153
34. Macauley, M. S., Crocker, P. R., and Paulson, J. C. (2014) Siglec-mediated regulation of immune cell function in disease. Nat Rev Immunol 14, 653-666
35. Lowell, C. A. (2011) Src-family and Syk kinases in activating and inhibitory pathways in innate immune cells: signaling cross talk. Cold Spring Harb Perspect Biol 3
36. Linnartz, B., Wang, Y., and Neumann, H. (2010) Microglial immunoreceptor tyrosine-based activation and inhibition motif signaling in neuroinflammation. Int J Alzheimers Dis 2010
37. Zhang, B., Gaiteri, C., Bodea, L. G., Wang, Z., McElwee, J., Podtelezhnikov, A. A., Zhang, C., Xie, T., Tran, L., Dobrin, R., Fluder, E., Clurman, B., Melquist, S., Narayanan, M., Suver, C., Shah, H., Mahajan, M., Gillis, T., Mysore, J., MacDonald, M. E., Lamb, J. R., Bennett, D. A., Molony, C., Stone, D. J., Gudnason, V., Myers, A. J., Schadt, E. E., Neumann, H., Zhu, J., and Emilsson, V. (2013) Integrated systems approach identifies genetic nodes and networks in late-onset Alzheimer's disease. Cell 153, 707-720
38. Wissfeld, J., Nozaki, I., Mathews, M., Raschka, T., Ebeling, C., Hornung, V., Brustle, O., and Neumann, H. (2021) Deletion of Alzheimer's disease-associated CD33 results in an inflammatory human microglia phenotype. Glia 69, 1393-1412
39. Lajaunias, F., Dayer, J. M., and Chizzolini, C. (2005) Constitutive repressor activity of CD33 on human monocytes requires sialic acid recognition and phosphoinositide 3-kinase-mediated intracellular signaling. Eur J Immunol 35, 243-251
40. Fu, A. K., Hung, K. W., Yuen, M. Y., Zhou, X., Mak, D. S., Chan, I. C., Cheung, T. H., Zhang, B., Fu, W. Y., Liew, F. Y., and Ip, N. Y. (2016) IL-33 ameliorates Alzheimer's disease-like pathology and cognitive decline. Proc Natl Acad Sci USA 113, E2705-2713
41. Kossman, S. E., Scheinberg, D. A., Jurcic, J. G., Jimenez, J., and Caron, P. C. (1999) A phase I trial of humanized monoclonal antibody HuM195 (anti-CD33) with low-dose interleukin 2 in acute myelogenous leukemia. Clin Cancer Res 5, 2748-2755
42. Saunders, K. O. (2019) Conceptual Approaches to Modulating Antibody Effector Functions and Circulation Half-Life. Front Immunol 10, 1296
43. Rankovic, Z. (2015) CNS drug design: balancing physicochemical properties for optimal brain exposure. J Med Chem 58, 2584-2608
44. Kouhi, A., Pachipulusu, V., Kapenstein, T., Hu, P., Epstein, A. L., and Khawli, L. A. (2021) Brain Disposition of Antibody-Based Therapeutics: Dogma, Approaches and Perspectives. Int J Mol Sci 22
45. Yu, Y. J., and Watts, R. J. (2013) Developing therapeutic antibodies for neurodegenerative disease. Neurotherapeutics 10, 459-472
46. Grabrucker, A. M., Ruozi, B., Belletti, D., Pederzoli, F., Fori, F., Vandelli, M. A., and Tosi, G. (2016) Nanoparticle transport across the blood brain barrier. Tissue Barriers 4, e1153568
47. Weber, F., Bohrmann, B., Niewoehner, J., Fischer, J. A. A., Rueger, P., Tiefenthaler, G., Moelleken, J., Bujotzek, A., Brady, K., Singer, T., Ebeling, M., Iglesias, A., and Freskgard, P. O. (2018) Brain Shuttle Antibody for Alzheimer's Disease with Attenuated Peripheral Effector Function due to an Inverted Binding Mode. Cell Rep 22, 149-162
48. Rosenblat, T. L., McDevitt, M. R., Mulford, D. A., Pandit-Taskar, N., Divgi, C. R., Panageas, K. S., Heaney, M. L., Chanel, S., Morgenstern, A., Sgouros, G., Larson, S. M., Scheinberg, D. A., and Jurcic, J. G. (2010) Sequential cytarabine and alpha-particle immunotherapy with bismuth-213-lintuzumab (HuM195) for acute myeloid leukemia. Clin Cancer Res 16, 5303-5311
49. Jurcic, J. G., Levy, M. Y., Park, J. H., Ravandi, F., Perl, A. E., Pagel, J. M., Smith, B. D., Estey, E. H., Kantarjian, H., Cicic, D., and Scheinberg, D. A. (2016) Phase I Trial of Targeted Alpha-Particle Therapy with Actinium-225 (Ac-225)-Lintuzumab and Low-Dose Cytarabine (LDAC) in Patients Age 60 or Older with Untreated Acute Myeloid Leukemia (AML). Blood 128

1. A method for treating or preventing Alzheimer's disease or reducing brain accumulation and/or persistence of AP plaques in a subject in need thereof, comprising administering to the subject a therapeutically effective amount of an anti-CD33 antibody, or an antigen binding fragment thereof, wherein the anti-CD33 antibody or antigen binding fragment includes an immunoglobulin heavy chain variable region (V_{H}) comprising SEQ ID NO: 1 and an immunoglobulin light chain variable region (V_{L}) comprising SEQ ID NO: 2.

2. (canceled)

3. The method of claim 1, wherein the anti-CD33 antibody or antigen binding fragment comprises a Fc domain of an isotype selected from the group consisting of IgG1, IgG2, IgG3, IgG4, IgA₁, IgA₂, IgM, IgD, and IgE, optionally wherein the anti-CD33 antibody or antigen binding fragment comprises an IgG1 constant region comprising one or more amino acid substitutions selected from the group consisting of N297A and K322A or wherein the anti-CD33 antibody or antigen binding fragment comprises an IgG4 constant region comprising a S228P mutation: or
wherein the Fc domain comprises a blood-brain barrier (BBB) target epitope, optionally wherein the BBB target epitope comprises an IgG constant region comprising a plurality of amino acid substitutions selected from the group consisting of:
(a) N384L, Q386L, P387V, E388W, N389V, N390G, D413A, R416T, and N421 W;
(b) N384Y, Q386T, P387V, E388W, N389S, N390H, D413S, R416E, and N421Y:
(c) N384Y, Q386T, P387E, E388W, N389S, N390Q, D413E, R416D, and N421H;
(d) N384V, Q386T, P387P, E388W, N389A, N390L, D413L, R416E, and N421 W;
(e) N384L, Q386H, P387V, E388W, N389A, N390V, D413P, R416T, and N421 W;
(f) N384Y, Q386T, P387E, E388W, N389S, N390S, D413T, R416E, and N421F:
(g) N384Y, Q386T, P387E, E388W, N389S, N390S, D413T, S415E, R416E, and N421F;
(h) E380W, N384Y, Q386T, P387E, E388W, N389S, N390S, D413T, S415E, R416E, and N421F;
(i) E380W, N384Y, Q386T, P387E, E388W, N389S, N390S, K392R, D413T, K414R, S415E, R416E, N421F, S424T and S426G; and
(j) E380L, N384Y, Q386T, P387E, E388W, N389S, N390S, D413T, S415E, R416E, and N421F; or
wherein the anti-CD33 antigen binding fragment is selected from the group consisting of Fab, F(ab′)₂, Fab′, scFv, and Fv; or
wherein the scFv comprises the amino acid sequence of SEQ ID NO: 3; or
wherein the anti-CD33 antibody or antigen binding fragment is conjugated to insulin, transferrin, an interleukin, albumin, a plasma protein, a lipoprotein, a RVG29 peptide, a Mini-Aβ4 (apamin) peptide, a shark antibody, an antibody that targets insulin receptor, an antibody that targets interleukin receptor, or an antibody that targets transferrin receptor; or
wherein the anti-CD33 antibody or antigen binding fragment is encapsulated by a nanoparticle or an exosome; or
wherein the anti-CD33 antibody or antigen binding fragment is a monoclonal antibody, a chimeric antibody, a humanized antibody, or a bispecific antibody; or
wherein administration of the anti-CD33 antibody or antigen binding fragment results in decreased cell surface expression of CD33 in microglia, increased Aβ uptake by microglia, and/or prolonged survival of the subject.

4. (canceled)

5. (canceled)

6. (canceled)

7. (canceled)

8. (canceled)

9. (canceled)

10. (canceled)

11. (canceled)

12. (canceled)

13. An engineered immune cell comprising:
(a) a neuronal antigen-specific receptor and/or a nucleic acid encoding the neuronal antigen-specific receptor; and
(b) an anti-CD33 antibody, or an antigen binding fragment thereof and/or a nucleic acid encoding the anti-CD33 antibody or antigen binding fragment, wherein the anti-CD33 antibody or antigen binding fragment includes an immunoglobulin heavy chain variable region (V_{H}) comprising SEQ ID NO: 1 and an immunoglobulin light chain variable region (V_{L}) comprising SEQ ID NO: 2.

14. The engineered immune cell of claim 13, wherein the neuronal antigen-specific receptor is a T cell receptor, a native cell receptor, a non-native cell receptor, or a chimeric antigen receptor (CAR); or
wherein the anti-CD33 antibody or antigen binding fragment is secreted, optionally wherein the nucleic acid encoding the anti-CD33 antibody or antigen binding fragment comprises a leader sequence for secretion of the anti-CD33 antibody or antigen binding fragment; or
wherein the anti-CD33 antibody or antigen binding fragment is a scFv, optionally wherein the scFv comprises the amino acid sequence of SEQ ID NO: 3; or
wherein the nucleic acid encoding the anti-CD33 antibody or antigen binding fragment comprises SEQ ID NO: 4; or
wherein the nucleic acid encoding the anti-CD33 antibody or antigen binding fragment is operably linked to a constitutive promoter, or a conditional promoter, optionally wherein the conditional promoter is inducible by binding of the neuronal antigen-specific receptor; or
wherein the neuronal antigen is GD2, GD3, GM1, NCAM, integrin 3, Thy-1, CD44, EGFRvIII, or PDGFR: or
wherein administration of the engineered immune cell results in decreased cell surface expression of CD33 in microglia, increased Aβ uptake by microglia, and/or prolonged survival of the subject.

15. (canceled)

16. (canceled)

17. (canceled)

18. (canceled)

19. (canceled)

20. (canceled)

21. (canceled)

22. (canceled)

23. The engineered immune cell of claim 14, wherein the chimeric antigen receptor comprises (i) an extracellular antigen binding domain; (ii) a transmembrane domain; and (iii) an intracellular domain.

24. The engineered immune cell of claim 23, wherein the extracellular antigen binding domain binds to the neuronal antigen; or
wherein the extracellular antigen binding domain comprises a single chain variable fragment (scFv) or a human scFv; or
wherein the extracellular antigen binding domain comprises a signal peptide that is covalently joined to the N-terminus of the extracellular antigen binding domain; or
wherein the transmembrane domain comprises a CD8 transmembrane domain; or
wherein the intracellular domain comprises one or more costimulatory domains, optionally wherein the one or more costimulatory domains are selected from a CD28 costimulatory domain, a CD3ζ chain, a 4-1BBL costimulatory domain, and any combination thereof; or
wherein the engineered immune cell is a lymphocyte, optionally wherein the lymphocyte is a T cell, a CD4+ T cell, a CD8+ T cell, a B cell, or a natural killer (NK) cell; or
wherein the engineered immune cell is derived from an autologous donor or an allogenic donor; or
wherein the engineered immune cell further comprises FoxP3 and/or a nucleic acid encoding FoxP3.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. A mixture of polypeptides comprising a first polypeptide comprising FoxP3 and a chimeric antigen receptor that specifically binds to a neuronal antigen, and a second polypeptide comprising an anti-CD33 antibody, or an antigen binding fragment thereof, wherein the anti-CD33 antibody or antigen binding fragment includes an immunoglobulin heavy chain variable region (V_{H}) comprising SEQ ID NO: 1 and an immunoglobulin light chain variable region (V_{L}) comprising SEQ ID NO: 2.

37. The mixture of polypeptides of claim 36, further comprising a self-cleaving peptide located between FoxP3 and the chimeric antigen receptor; or
wherein the self-cleaving peptide is a P2A or a T2A self-cleaving peptide; or
wherein the second polypeptide comprises a leader sequence for secretion of the anti-CD33 antibody or antigen binding fragment; or
wherein the second polypeptide comprises a scFv, optionally wherein the scFv comprises the amino acid sequence of SEQ ID NO: 3.

38. (canceled)

39. (canceled)

40. (canceled)

41. The mixture of polypeptides of claim 36, wherein the chimeric antigen receptor comprises (i) an extracellular antigen binding domain; (ii) a transmembrane domain; and (iii) an intracellular domain.

42. The mixture of polypeptides of claim 41, wherein the extracellular antigen binding domain binds to a neuronal antigen-specific receptor, optionally wherein the neuronal antigen is GD2, GD3, GM1, NCAM, integrin 3, Thy-1, CD44, EGFRvIII, or PDGFR; or
wherein the extracellular antigen binding domain comprises a scFv; or
wherein the transmembrane domain comprises a CD8 transmembrane domain; or
wherein the intracellular domain comprises one or more costimulatory domains, optionally wherein the one or more costimulatory domains are selected from a CD28 costimulatory domain, a CD3ζ chain, a 4-1BBL costimulatory domain, and any combination thereof.

43. (canceled)

44. (canceled)

45. (canceled)

46. (canceled)

47. (canceled)

48. A nucleic acid encoding the mixture of polypeptides of claim 41.

49. The nucleic acid of claim 48, wherein the nucleic acid encoding the mixture of polypeptides is operably linked to a constitutive promoter, or a conditional promoter, optionally wherein the conditional promoter is inducible by binding of the neuronal antigen-specific receptor.

50. (canceled)

51. (canceled)

52. A vector comprising the nucleic acid of claim 48, optionally wherein the vector is a viral vector, a retroviral vector, or a plasmid.

53. (canceled)

54. A host cell comprising the vector of claim 52.

55. A method for treating or preventing Alzheimer's disease in a subject in need thereof, comprising administering to the subject a therapeutically effective amount of the engineered immune cell of claim 13.

56. The method of claim 1, wherein the subject is suspected of having, is at risk for, or is diagnosed as having late onset Alzheimer's disease, early onset Alzheimer's disease, or intermediate onset Alzheimer's disease; or
wherein the subject exhibits one or more signs or symptoms of Alzheimer's disease, optionally wherein the one or more signs or symptoms of Alzheimer's disease are selected from the group consisting of: cognitive dysfunction or decline; memory loss; agitation; mood swings; impaired judgment; dementia; difficulty with abstract thinking; difficulty with familiar tasks; disorientation; diminished communication skills; repetitive speech or actions; impaired visuospatial abilities; impaired speaking, reading, and writing; withdrawal; depression; loss of recognition; loss of motor skills and sense of touch; delusions; paranoia; verbal or physical aggression; and sleep disorders; or
wherein the subject exhibit mutations in one or more genes selected from the group consisting of APP, PS1, PS2, APOE4, CD33, CLU, BIN1, PICALM, CR1, CD2AP, EPHA1, ABCA7, MS4A4A/MS4A6E and TREM2; or
wherein the anti-CD33 antibody or antigen binding fragment is administered systemically, intravenously, subcutaneously, intraperitoneally, intradermally, iontophoretically, transmucosally, intrathecally, intramuscularly, intracerebrally, or intracerebroventricularly.

57. (canceled)

58. (canceled)

59. (canceled)

60. (canceled)

61. The method of claim 55, wherein the engineered immune cell is administered systemically, intravenously, subcutaneously, intraperitoneally, intradermally, iontophoretically, transmucosally, intrathecally, intramuscularly, intracerebrally, or intracerebroventricularly; or
wherein the method further comprises separately, sequentially or simultaneously administering at least one additional therapeutic agent to the subject, optionally wherein the at least one additional therapeutic agent is selected from the group consisting of donepezil, galantamine, memantine, rivastigmine, memantine extended-release and donepezil (Namzaric), aducanumab, solanezumab, insulin, verubecestat, AADvac1, CSP-1103, and intepirdine.

62. The method of claim 1, further comprising separately, sequentially or simultaneously administering at least one additional therapeutic agent to the subject, optionally wherein the at least one additional therapeutic agent is selected from the group consisting of donepezil, galantamine, memantine, rivastigmine, memantine extended-release and donepezil (Namzaric), aducanumab, solanezumab, insulin, verubecestat, AADvac1, CSP-1103, and intepirdine.

63. (canceled)

64. (canceled)

65. A method of preparing immune cells for adoptive cell therapy comprising:
isolating immune cells from a donor subject;
transducing the immune cells with the vector of claim 52; and
administering the transduced cells to a recipient subject.

66. The method of claim 65, wherein the donor subject and the recipient subject are the same or different; or
wherein the immune cells isolated from the donor subject comprise one or more lymphocytes, optionally wherein the one or more lymphocytes is a T cell, a CD8⁺ cytotoxic T cell, a CD4⁺ T cell, a B cell, a tumor infiltrating lymphocyte, or a natural killer cell; or
wherein the T cell comprises a native T cell receptor (TCR), a non-native TCR, or a chimeric antigen receptor (CAR).

67. (canceled)

68. (canceled)

69. (canceled)

70. (canceled)
